SEQUENCE LISTING (3 sequences)

SEQ ID NO: 1 - RNA, 21 nt, synthetic construct
  misc_feature 1..21: note = siRNA sense
  modified_base 4..5: mod_base = OTHER; note = 2'-fluoro-modified nucleotide
  modified_base 7..10: mod_base = OTHER; note = 2'-fluoro-modified nucleotide
  modified_base 14..17: mod_base = OTHER; note = 2'-fluoro-modified nucleotide
  modified_base 19: mod_base = OTHER; note = 2'-fluoro-modified nucleotide
  misc_feature 20..21: note = n is deoxythymidine
  misc_feature 20..21: note = phosphorothioate linkage
  source 1..21: mol_type = other RNA
ggatcatctcaagtcttacnn

SEQ ID NO: 2 - RNA, 21 nt, synthetic construct
  misc_feature 1..21: note = siRNA antisense
  modified_base 2: mod_base = OTHER; note = 2'-fluoro-modified nucleotide
  modified_base 7..9: mod_base = OTHER; note = 2'-fluoro-modified nucleotide
  modified_base 14: mod_base = OTHER; note = 2'-fluoro-modified nucleotide
  modified_base 17..19: mod_base = OTHER; note = 2'-fluoro-modified nucleotide
  misc_feature 20..21: note = n is deoxythymidine
  misc_feature 20..21: note = phosphorothioate linkage
  source 1..21: mol_type = other RNA
gtaagacttgagatgatccnn

SEQ ID NO: 3 - protein, 8 aa, synthetic construct
  REGION 1..8: note = OVA peptide
SIINFEKL

(19) United States
(12) Patent Application Publication
(10) Pub. No.: US 2026/0124154 A1
(43) Pub. Date: May 7, 2026

(54) LIPID NANOPARTICLE LYOPHILIZED COMPOSITION

(71) Applicants: NOF CORPORATION, Tokyo (JP); NATIONAL UNIVERSITY CORPORATION CHIBA UNIVERSITY, Chiba-shi (JP)

(72) Inventors: Kota TANGE, Kawasaki (JP); Yuta NAKAI, Kawasaki (JP); Shinya TAMAGAWA, Kawasaki (JP); Sakura TANEICHI, Kawasaki (JP); Ayano KOIKE, Kawasaki (JP); Hidetaka AKITA, Chiba (JP); Hiroki TANAKA, Chiba (JP); Daiki SHIRANE, Chiba (JP); Shinya HAGIWARA, Chiba (JP)

(73) Assignees: NOF CORPORATION, Tokyo (JP); NATIONAL UNIVERSITY CORPORATION CHIBA UNIVERSITY, Chiba-shi (JP)

(21) Appl. No.: 19/436,678

(22) Filed: Dec. 30, 2025

Related U.S. Application Data
(63) Continuation-in-part of application No. 17/762,259, now Pat. No. 12,551,445.

(30) Foreign Application Priority Data
Sep. 26, 2019 (JP) 2019-176253

(51) Int. Cl. A61K 9/51; A61K 9/19; C12N 15/88

(52) U.S. Cl. CPC A61K 9/5123; A61K 9/19; A61K 9/5192; C12N 15/88

(57) ABSTRACT

A lyophilized composition capable of encapsulating any nucleic acid with high efficiency and easily is provided. A lyophilized composition of lipid nanoparticles not containing a nucleic acid but containing an ionic lipid, a sterol, a PEG lipid, an acidic buffer component that shows a buffering action at pH 1-6, and a cryoprotectant, wherein a weight ratio of the cryoprotectant and a total lipid is 10:1-1000:1.

### CROSS-REFERENCE TO RELATED APPLICATIONS

This patent application is a continuation-in-part of co-pending U.S. patent application Ser. No. 17/762,259, filed on Mar. 21, 2022, which is the U.S. national phase of International Patent Application No. PCT/JP2020/036196, filed on Sep. 25, 2020, which claims the benefit of Japanese Patent Application No. 2019-176253 filed on Sep. 26, 2019, which are incorporated by reference in their entireties herein.

### INCORPORATION-BY-REFERENCE OF MATERIAL ELECTRONICALLY SUBMITTED

Incorporated by reference in its entirety herein is a computer-readable nucleotide/amino acid sequence listing submitted concurrently herewith and identified as follows: 10,642 bytes Extensible Markup Language (xml) file named “P11805WOUS-01.xml,” created Dec. 8, 2025.

### TECHNICAL FIELD

The present invention relates to a lyophilized composition of lipid nanoparticles not containing a nucleic acid and a method for producing nucleic acid-encapsulating lipid nanoparticles by using same.

### BACKGROUND ART

For practicalization of nucleic acid therapy using oligonucleic acids such as siRNA, and gene therapy using mRNA, pDNA, and the like, an effective and safe nucleic acid delivery carrier is demanded. While virus vectors are nucleic acid delivery carriers with good expression efficiency, the development of non-viral nucleic acid delivery carriers that can be used more safely is ongoing.

Since cationic liposomes using cationic lipids with quaternary amine are positively charged, they can form a complex (lipoplex) by electrostatic interaction with negatively-charged nucleic acids, and can deliver nucleic acids into cells. Utilizing the electrostatic interaction of quaternary amine with nucleic acid, it is also possible to prepare a lyophilized composition of cationic liposomes not containing nucleic acids and form a lipoplex by rehydration with an aqueous solution of nucleic acid. Thus, it has been shown that use thereof as a gene transfer reagent is available (Patent Literatures 1 and 2).

However, it is difficult to control the particle size of lipoplex produced by such method, and cytotoxicity derived from positively-charged cationic lipids becomes a problem.

Therefore, lipid nanoparticles (LNP) using ionic lipids having a tertiary amine—which is positively charged under acidic conditions and has no electric charge under near neutral conditions—in the molecule were developed, and have become non-viral nucleic acid delivery carriers most generally used at present (Non Patent Literature 1).

As lipid nanoparticles using an ionic lipid having a tertiary amine in the molecule, an example also exists in which a degradable group is added to the ionic lipid (Patent Literature 3).

As described, various non-viral carriers have been developed. However, since nucleic acids are generally unstable compounds, there are still problems with their stability as pharmaceutical preparations.

As one of the methods for improving the stability as a pharmaceutical preparation, attempts have been made to lyophilize lipid nanoparticles encapsulating nucleic acid and rehydrate them at the time of use to reconstitute the lipid nanoparticles (Patent Literature 4 and Non Patent Literature 2).

Although these methods are useful as a method for enhancing the storage stability of lipid nanoparticles encapsulating a specific nucleic acid, there is a problem as a method for more easily encapsulating any nucleic acid in lipid nanoparticles.

As a method for more easily encapsulating any nucleic acid in lipid nanoparticles, a method of preparing a lyophilized composition not containing a nucleic acid and then rehydrating same with an aqueous solution of nucleic acid, like the methods described in Patent Literatures 1 and 2, can be mentioned.

However, lipid nanoparticles obtained using ionic lipid having tertiary amine in the molecule do not electrostatically interact with nucleic acid because the surface charge after preparation is weakly negative to neutral. Thus, the method of preparing a lyophilized composition not containing a nucleic acid and then rehydrating same with an aqueous solution of nucleic acid, disclosed in Patent Literatures 1 and 2, cannot prepare nucleic acid-encapsulating lipid nanoparticles.

As described above, no means were available for easily encapsulating any nucleic acid in lipid nanoparticles with a high nucleic acid encapsulation rate.

### CITATION LIST

### Patent Literatures

### [PTL 1]

JP-B-4919397

### [PTL 2]

JP-B-4598908

### [PTL 3]

JP-B-6093710

### [PTL 4]

WO 2017/218704

### Non Patent Literatures

### [NPL 1]

Gene Therapy 6:271-281, 1999

### [NPL 2]

Biol. Pharm. Bull. 41, 1291-1294(2018)

### SUMMARY OF INVENTION

### Technical Problem

The problems of the present invention are provision of a lyophilized composition capable of encapsulating any nucleic acid with high efficiency and easily, which could not be achieved by the prior art, and a production method of nucleic acid-encapsulating lipid nanoparticles by using the composition.

### Solution to Problem

The present inventors have conducted intensive studies in view of the above-mentioned problems and found that any nucleic acid can be encapsulated with high efficiency and easily by preparing lipid nanoparticles not containing nucleic acid in an acidic buffer, further adding a cryoprotectant, lyophilizing same, and then rehydrating same with an aqueous solution containing nucleic acid. Furthermore, they conducted experiments to transfer gene into cells by using the lipid nanoparticles prepared by this method, and found that uniform gene transfer into cells is possible by increasing the concentration of the cryoprotectant before lyophilizing, which resulted in the completion of the present invention.

Accordingly, the present invention encompasses the following.
[1]A lyophilized composition of lipid nanoparticles not comprising a nucleic acid but comprising an ionic lipid, a sterol, a PEG lipid, an acidic buffer component that shows a buffering action at pH 1-6, and a cryoprotectant, wherein a weight ratio of the cryoprotectant and a total lipid is 10:1-1000:1.
[2] The lyophilized composition of [1], further comprising a phospholipid.
[3] The lyophilized composition of [1] or [2], wherein the weight ratio of the cryoprotectant and the total lipid is 30:1-1000:1.
[4] The lyophilized composition of any one of [1] to [3], wherein a concentration of the cryoprotectant in the composition before lyophilizing is 80-800 mg/mL.
[5] The lyophilized composition of any one of [1] to [4], wherein a concentration of the cryoprotectant in the composition before lyophilizing is 160-800 mg/mL.
[6] The lyophilized composition of any one of [1] to [5], wherein the ionic lipid is a compound represented by the formula (1):

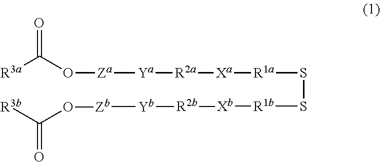

(in the formula (1), R^{1a} and R^{1b} are each independently an alkylene group having 1 to 6 carbon atoms,
   X^{a} and X^{b} are each independently an acyclic alkyl tertiary amino group having 1 to 6 carbon atoms and 1 tertiary amino group, or a cyclic alkylene tertiary amino group having 2 to 5 carbon atoms and 1 or 2 tertiary amino groups,
   R^{2a} and R^{2b} are each independently an alkylene group having 1 to 8 carbon atoms or an oxydialkylene group having 2 to 8 carbon atoms,
   Y^{a} and Y^{b} are each independently an ester bond, an amide bond, a carbamate bond, an ether bond, or a urea bond,
   Z^{a} and Z^{b} are each independently a divalent group derived from an aromatic compound having 3 to 16 carbon atoms, at least one aromatic ring, and optionally having a heteroatom,
   R^{3a} is
(ia) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group),
(iia) a monovalent group having 10 to 50 carbon atoms and at least two carbonyl groups (excluding a monovalent group containing a residue of a liposoluble vitamin having a hydroxy group and a residue of a sterol derivative having a hydroxy group),
(iiia) a monovalent group represented by the formula (2):

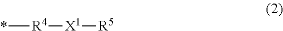

(in the formula (2), * is a bonding position,
   R⁴ is an alkylene group having 1 to 10 carbon atoms,
   X¹ is a carbamate bond, a carbonate bond, or an amide bond, and
   R⁵ is an alkyl group having 1 to 25 carbon atoms, and R⁵ is optionally substituted by a substituent selected from the group consisting of a halogen atom and a hydroxy group),
(iva) a monovalent group represented by the formula (3):

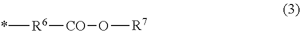

(in the formula (3), * is a bonding position,
   R⁶ is an alkylene group having 1 to 10 carbon atoms, and
   R⁷ is an alkyl group having 1 to 25 carbon atoms and substituted by at least one halogen atom),
(va) a monovalent group represented by the formula (4):

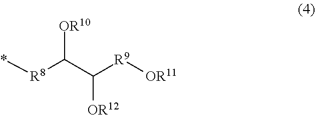

(in the formula (4), * is a bonding position,
   R⁸ and R⁹ are each independently an alkylene group having 1 to 10 carbon atoms, an alkenediyl group having 2 to 10 carbon atoms, or an alkynediyl group having 2 to 10 carbon atoms,
   R¹⁰ to R¹² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(via) a monovalent group represented by the formula (5):

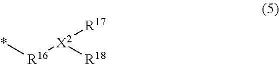

(in the formula (5), * is a bonding position,
   X² is a nitrogen atom or a trivalent group represented by the formula (6)

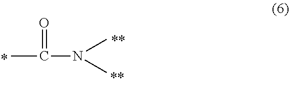

(in the formula (6), * is a bonding position with R¹⁶, and
   ** is a bonding position with R¹⁷ or R¹⁸),
   when X² is a nitrogen atom, R¹⁶ is an alkylene group having 1 to 10 carbon atoms, an alkenediyl group having 2 to 10 carbon atoms, or an alkynediyl group having 2 to 10 carbon atoms, and R¹⁶ is optionally substituted by a substituent selected from the group consisting of a halogen atom and a hydroxy group,
   when X² is a trivalent group represented by the formula (6), R¹⁶ is an alkylene group having 1 to 10 carbon atoms, and R¹⁶ is optionally substituted by a substituent selected from the group consisting of a halogen atom and a hydroxy group,
   when X² is a nitrogen atom, R¹⁷ and R¹⁸ are each independently an alkyl group having 1 to 10 carbon atoms, an alkenyl group having 2 to 10 carbon atoms, or an alkynyl group having 2 to 10 carbon atoms, and R¹⁷ and R¹⁸ are each independently optionally substituted by a substituent selected from the group consisting of a halogen atom and a hydroxy group, and
   when X² is a trivalent group represented by the formula (6), R¹⁷ and R¹⁸ are each independently an alkyl group having 1 to 10 carbon atoms, and R¹⁷ and R¹⁸ are each independently optionally substituted by a substituent selected from the group consisting of a halogen atom and a hydroxy group),
(viia) a monovalent group represented by the formula (7):

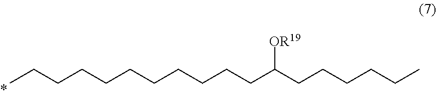

(in the formula (7), * is a bonding position, and
   R¹⁹ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group), or a *—CO—R²⁰ group (wherein * is a bonding position, and R²⁰ is an alkyl group having 1 to 9 carbon atoms)),
(viiia) a monovalent group represented by the formula (8):

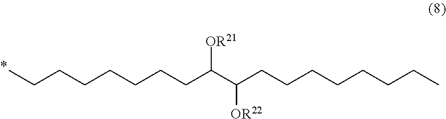

(in the formula (8), * is a bonding position, and
   R²¹ and R²² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(ixa) a monovalent group represented by the formula (9):

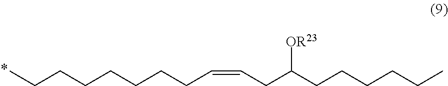

(in the formula (9), * is a bonding position, and
   R²³ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(xa) a monovalent group represented by the formula (10):

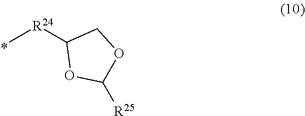

(in the formula (10), * is a bonding position,
   R²⁴ is an alkylene group having 1 to 10 carbon atoms, an alkenediyl group having 2 to 10 carbon atoms, or an alkynediyl group having 2 to 10 carbon atoms, and
   R²⁵ is an alkyl group having 1 to 30 carbon atoms, an alkenyl group having 2 to 30 carbon atoms, or an alkynyl group having 2 to 30 carbon atoms),
(xia) a monovalent group represented by the formula (11):

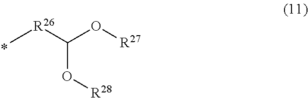

(in the formula (11), * is a bonding position, and
   R²⁶ is an alkylene group having 1 to 10 carbon atoms, an alkenediyl group having 2 to 10 carbon atoms, or an alkynediyl group having 2 to 10 carbon atoms, and
   R²⁷ and R²⁸ are each independently an alkyl group having 1 to 10 carbon atoms, an alkenyl group having 2 to 10 carbon atoms, or an alkynyl group having 2 to 10 carbon atoms), or
(xiia) a monovalent group represented by the formula (12):

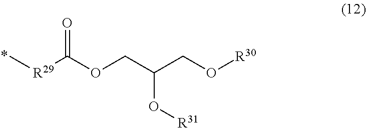

(in the formula (12), * is a bonding position,
   R²⁹ is an alkylene group having 1 to 10 carbon atoms, and
   R³⁰ and R³¹ are each independently an alkyl group having 1 to 10 carbon atoms),
   R^{3b} is
(ib) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group),
(iib) a monovalent group having 10 to 50 carbon atoms and at least two carbonyl groups (excluding a monovalent group containing a residue of a liposoluble vitamin having a hydroxy group and a residue of a sterol derivative having a hydroxy group),
(iiib) a monovalent group represented by the formula (2),
(ivb) a monovalent group represented by the formula (3),
(vb) a monovalent group represented by the formula (4),
(vib) a monovalent group represented by the formula (5),
(viib) a monovalent group represented by the formula (7),
(viiib) a monovalent group represented by the formula (8),
(ixb) a monovalent group represented by the formula (9),
(xb) a monovalent group represented by the formula (10),
(xib) a monovalent group represented by the formula (11),
(xiib) a monovalent group represented by the formula (12),
(xiiib) a monovalent group which is an alkyl group having 1 to 30 carbon atoms, an alkenyl group having 2 to 20 carbon atoms, or an alkynyl group having 2 to 20 carbon atoms, in which one ethylene group in the alkyl group is optionally replaced by one ester bond, or
(xivb) an R^{3c}—CO—(CH₂)ₚ— group (wherein R^{3c} is a residue of a liposoluble vitamin having a hydroxy group or a residue of a sterol derivative having a hydroxy group, and p is an integer of 1 to 8), and
   R^{3a} and R^{3b} may be the same or different).
[7] The lyophilized composition of any one of [1] to [5], wherein the ionic lipid is a compound represented by the formula (2A):

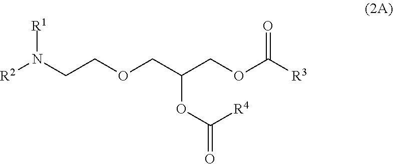

(in the formula (2A), R¹ and R² are each independently an alkyl group having 1 to 6 carbon atoms, or any carbon atoms of R¹ and R² are optionally bonded to each other to form a heterocycle containing nitrogen atom, and
   R³ and R⁴ are each independently an aliphatic hydrocarbon group having 1 to 35 carbon atoms optionally substituted by a substituent selected from Substituent Group α,
   provided that at least one selected from the group consisting of R³ and R⁴ is an aliphatic hydrocarbon group having 5 to 35 carbon atoms optionally substituted by a substituent selected from Substituent Group α, wherein Substituent Group α consists of a hydroxy group, an alkoxy group, a sulfanyl group, an alkylthio group, and an alkoxycarbonyl group).
[8] The lyophilized composition of any one of [1] to [7], wherein the cryoprotectant is disaccharide.
[9] The lyophilized composition of any one of [1] to [7], wherein the cryoprotectant is sucrose.
[10]A method for producing a nucleic acid-encapsulating lipid nanoparticle, comprising the following steps:
a) a step of mixing an alcohol solution containing an ionic lipid, a sterol, and a PEG lipid, and an acidic buffer showing a buffering action at pH 1-6 to prepare a suspension of lipid nanoparticles not containing a nucleic acid,
b) a step of mixing the suspension of the lipid nanoparticles not containing a nucleic acid and a cryoprotectant to give a mixture with pH 1-6 and containing the cryoprotectant at 80-800 mg/mL,
c) a step of lyophilizing the mixture obtained in step b to give a lyophilized composition,
d) a step of mixing the lyophilized composition and an aqueous solution containing a nucleic acid and optionally containing alcohol at 0-25 v/v %, and optionally incubating the mixture at 0-95° C. for 0-60 min to give nucleic acid-encapsulating lipid nanoparticles, and
e) a step of exchanging an external aqueous phase of the obtained nucleic acid-encapsulating lipid nanoparticles with a neutral buffer by dialysis, ultrafiltration, or dilution.
[11] The method of [10], wherein the step a further comprises a step of exchanging the external aqueous phase with another acidic buffer showing a buffering action at pH 1-6, by dialysis, ultrafiltration, or dilution, after preparing the suspension of lipid nanoparticles.
[12] The method of [10] or [11], wherein the alcohol solution further comprises a phospholipid in step a.
[13] The method of any one of [10] to [12], wherein a concentration of the cryoprotectant in the mixture in step b is 160-800 mg/mL.
[14] The method of any one of [10] to [13], wherein the ionic lipid is a compound represented by the aforementioned formula (1).
[15] The method of any one of [10] to [13], wherein the ionic lipid is a compound represented by the aforementioned formula (2A).
[16] The method of any one of [10] to [15], wherein the cryoprotectant is disaccharide.
[17] The method of any one of [10] to [15], wherein the cryoprotectant is sucrose.
[18]A method for producing a lyophilized composition of lipid nanoparticles, comprising a step of lyophilizing a composition of lipid nanoparticles not containing a nucleic acid but containing an ionic lipid, a sterol, a PEG lipid, an acidic buffer that shows a buffering action at pH 1-6, and a cryoprotectant at 80-800 mg/mL.
[19] The method of [18], wherein the composition of lipid nanoparticles further comprises a phospholipid.
[20] The method of [18] or [19], wherein a concentration of the cryoprotectant in the composition before lyophilizing is 160-800 mg/mL.
[21] The method of any one of [18] to [20], wherein the ionic lipid is a compound represented by the aforementioned formula (1).
[22] The method of any one of [18] to [20], wherein the ionic lipid is a compound represented by the aforementioned formula (2A).
[23] The method of any one of [18] to [22], wherein the cryoprotectant is disaccharide.
[24] The method of any one of [18] to [22], wherein the cryoprotectant is sucrose.
[25]A nucleic acid-introducing agent comprising the lyophilized composition of any one of [1] to [9].
[26]A method for introducing a nucleic acid into a cell in vitro, comprising bringing the nucleic acid-introducing agent of [25] encapsulating the nucleic acid into contact with the cell.
[27]A method for introducing a nucleic acid into a target cell, comprising administering the nucleic acid-introducing agent of [25] encapsulating the nucleic acid to a living organism to allow for delivery of the nucleic acid to the cell.
[28]A method for introducing a nucleic acid into a cell, comprising the following steps:
a) a step of mixing an alcohol solution containing an ionic lipid, a sterol, and a PEG lipid, and an acidic buffer showing a buffering action at pH 1-6 to prepare a suspension of lipid nanoparticles not containing a nucleic acid,
b) a step of mixing the suspension of the lipid nanoparticles not containing a nucleic acid and a cryoprotectant to give a mixture with pH 1-6 and containing the cryoprotectant at 80-800 mg/mL,
c) a step of lyophilizing the mixture obtained in step b to give a lyophilized composition,
d) a step of mixing the lyophilized composition and an aqueous solution containing the nucleic acid and optionally containing alcohol at 0-25 v/v %, and optionally incubating the mixture at 0-95° C. for 0-60 min to give nucleic acid-encapsulating lipid nanoparticles,
e) a step of exchanging an external aqueous phase of the obtained nucleic acid-encapsulating lipid nanoparticles with a neutral buffer by dialysis, ultrafiltration, or dilution, and
f) a step of bringing the obtained nucleic acid-encapsulating lipid nanoparticles into contact with the cell in vitro.
[29] The method of [28], wherein the step a further comprises a step of exchanging the external aqueous phase with another acidic buffer showing a buffering action at pH 1-6, by dialysis, ultrafiltration, or dilution, after preparing the suspension of lipid nanoparticles.
[30]A method for introducing a nucleic acid into a target cell, comprising the following steps:
a) a step of mixing an alcohol solution containing an ionic lipid, a sterol, and a PEG lipid, and an acidic buffer showing a buffering action at pH 1-6 to prepare a suspension of lipid nanoparticles not containing a nucleic acid,
b) a step of mixing the suspension of the lipid nanoparticles not containing a nucleic acid and a cryoprotectant to give a mixture with pH 1-6 and containing the cryoprotectant at 80-800 mg/mL,
c) a step of lyophilizing the mixture obtained in step b to give a lyophilized composition,
d) a step of mixing the lyophilized composition and an aqueous solution containing the nucleic acid and optionally containing alcohol at 0-25 v/v %, and optionally incubating the mixture at 0-95° C. for 0-60 min to give nucleic acid-encapsulating lipid nanoparticles,
e) a step of exchanging an external aqueous phase of the obtained nucleic acid-encapsulating lipid nanoparticles with a neutral buffer by dialysis, ultrafiltration, or dilution, and
f) a step of administering the obtained nucleic acid-encapsulating lipid nanoparticles to a living organism to allow so for delivery of the nucleic acid to the target cell.
[31] The method of [30], wherein the step a further comprises a step of exchanging the external aqueous phase with another acidic buffer showing a buffering action at pH 1-6, by dialysis, ultrafiltration, or dilution, after preparing the suspension of lipid nanoparticles.

### Advantageous Effects of Invention

The present invention relates to a lyophilized composition of lipid nanoparticles not containing a nucleic acid, and a preparation method of lipid nanoparticles by using same.

The lyophilized composition of the present invention can prepare lipid nanoparticles containing any nucleic acid highly efficiently and easily by rehydration with an aqueous solution containing a nucleic acid.

The preparation method of lipid nanoparticles of the present invention can further increase the encapsulation rate of nucleic acid by incubating the lyophilized composition of the present invention by adding an aqueous solution of any nucleic acid, and further, alcohol.

When gene transfer is performed using lipid nanoparticles prepared by the method of the present invention, the gene can be uniformly introduced into cells. Further, the lipid nanoparticles prepared by the method of the present invention have a particle size of 50-200 nm and a narrow particle size distribution, which is advantageous for gene transfer in vivo.

### BRIEF DESCRIPTION OF DRAWINGS

FIG. 1 shows evaluation of the influence of the sucrose concentration on the uniformity of gene transfer into cells.

FIG. 2 shows evaluation of the influence of the sucrose concentration on the expression intensity of gene transfer into cells.

FIG. 3 shows evaluation of the influence of the lipid composition when DOPC was used as a phospholipid, on the efficiency of gene transfer into cells.

FIG. 4 shows evaluation of the influence of the lipid composition when POPC was used as a phospholipid, on the efficiency of gene transfer into cells.

FIG. 5 shows evaluation of the influence of the lipid composition when DOPE was used as a phospholipid, on the efficiency of gene transfer into cells.

FIG. 6 shows evaluation of the influence of the lipid composition when POPE was used as a phospholipid, on the efficiency of gene transfer into cells.

FIG. 7 shows evaluation of in vivo mRNA expression efficiency in mouse.

FIG. 8 shows evaluation of in vivo CTL activity in mouse.

FIG. 9 shows comparison of particles prepared by a conventional method and the particles of the present invention in the efficiency of gene transfer into cells.

FIG. 10 shows comparison of particles prepared by a conventional method and the particles of the present invention in the in vivo mRNA expression efficiency in mouse.

FIG. 11 shows evaluation of the influence of the lipid composition when No. 1, 3, 4, 5, or 7 was used as an ionic lipid, on the efficiency of gene transfer into cells.

FIG. 12 shows evaluation of the influence of the lipid composition when No. 8, 9, 10, 11, or 16 was used as an ionic lipid, on the efficiency of gene transfer into cells.

FIG. 13 shows evaluation of the influence of the lipid composition when No. 21, 22, 25, or a combination of SS-OP and SS-EC was used as an ionic lipid, on the efficiency of gene transfer into cells.

FIG. 14 shows evaluation of the influence of the lipid composition when No. 30, 31, 32, 33, or 34 was used as an ionic lipid, on the efficiency of gene transfer into cells.

FIG. 15 shows evaluation of the influence of the lipid composition when No. 35, 36, or 37 was used as an ionic lipid, on the efficiency of gene transfer into cells.

### DESCRIPTION OF EMBODIMENTS

While the embodiments of the present invention are explained in the following, the present invention is not limited thereto.

The present invention relates to a lyophilized composition of lipid nanoparticles not containing a nucleic acid but containing an ionic lipid, a sterol, a PEG lipid, an acidic buffer component that shows a buffering action at pH 1-6, and a cryoprotectant, wherein a weight ratio of the cryoprotectant and a total lipid is 10:1-1000:1.

A lipid nanoparticle means a particle having a membrane structure wherein the hydrophilic groups of amphiphilic lipid are arranged in the interface, facing the aqueous phase side. The “amphiphilic lipid” means a lipid having both a hydrophilic group showing hydrophilicity, and a hydrophobic group showing hydrophobicity. Examples of the amphiphilic lipid include ionic lipid, phospholipid, PEG lipid, and the like.

The lipid nanoparticles of the present invention contain ionic lipid, a sterol, and PEG lipid as substances constituting a membrane, and may further contain a phospholipid. The particle size of the lipid nanoparticles is not particularly limited, and is preferably 10 nm-500 nm, more preferably 30 nm-300 nm. The particle size can be measured by using a particle size distribution measuring device such as Zetasizer Nano (Malvern) or the like. The particle size of the lipid nanoparticles can be appropriately adjusted by the method for preparing the lipid nanoparticles. In the present invention, the particle size means an average particle size (number average) measured by a dynamic light scattering method.

In the present invention, the “total lipid” means the total amount of lipid. Examples of the lipid include ionic lipids, sterols, PEG lipids, and phospholipids.

In the present invention, “not containing nucleic acid” or “nucleic acid-free” means that nucleic acid is substantially not contained, and that the nucleic acid content is below the detection limit.

In the present invention, the “nucleic acid-encapsulating lipid nanoparticle” means a lipid nanoparticle in which a nucleic acid is encapsulated inside the lipid nanoparticle.

The lyophilized composition of the present invention is obtained by further adding a cryoprotectant to lipid nanoparticles that are obtained by dissolving an ionic lipid, a sterol, and a PEG lipid or further a phospholipid in a water-soluble organic solvent, and mixing same with an acidic buffer to induce organization, and lyophilizing the mixture. Examples of the water-soluble organic solvent include alcohols such as tert-butanol, ethanol and the like.

### Ionic Lipid

The ionic lipid that can be used in the present invention may be any as long as it is composed of a tertiary amino group and a hydrophobic group and can constitute lipid nanoparticles.

Specific examples of the ionic lipid include 1,2-dioleoyloxy-3-dimethylaminopropane (DODAP), 1,2-dioleyloxy-3-dimethylaminopropane (DODMA), 1,2-dilinoleyloxy-3-dimethylaminopropane (DLinDMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA or MC3), and compounds of the following formula (1) or the formula (2A).

The formula (1)

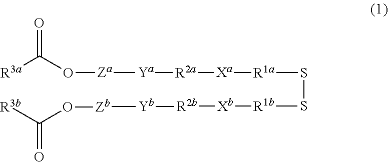

First, alkylene groups and the like included in the formula (1) are explained.

In the present specification, the alkylene group may be linear or branched. Examples of the alkylene group include methylene group, ethylene group, trimethylene group (—(CH₂)₃—), propylene group (—CH(CH₃)CH₂—, —CH₂CH(CH₃)—), tetramethylene group (—(CH₂)₄—), butylene group (—CH(C₂H₅)CH₂—, —CH₂CH(C₂H₅)—), pentamethylene group (—(CH₂)₅—), hexamethylene group (—(CH₂)₆—), heptamethylene group (—(CH₂)₇—), octamethylene group (—(CH₂)₈—), nonamethylene group (—(CH₂)₉—), and decamethylene group (—(CH₂)₉—) (in the aforementioned formulas, “—” is a single bond).

In the present specification, the “alkenediyl group” refers to a divalent group having a structure obtained by removing two hydrogen atoms from an alkene. In the present specification, the alkenediyl group may be linear or branched. The number of olefinic carbon-carbon double bonds in the alkene or alkenediyl group may be one or two or more. Examples of the alkenediyl group include ethenediyl group, propenediyl group, butenediyl group, pentenediyl group, hexenediyl group, heptenediyl group, octenediyl group, nonenediyl group, and decenediyl group. In the present specification, “compound name+diyl group (e.g., ethenediyl group)” refers to a divalent group having a structure obtained by removing two hydrogen atoms from the aforementioned compound.

In the present specification, the “alkynediyl group” means a divalent group having a structure obtained by removing two hydrogen atoms from an alkyne. In the present specification, the alkynediyl group may be linear or branched. In the present specification, the number of carbon-carbon triple bonds in the alkyne or alkynediyl group may be one or two or more. Examples of the alkynediyl group include ethynediyl group, propynediyl group, butynediyl group, pentynediyl group, hexynediyl group, heptynediyl group, octynediyl group, noninediyl group, and decynediyl group.

In the present specification, the “oxydialkylene group” means a divalent group having a structure in which two alkylene groups are bonded via an oxy group (—O—) (in the aforementioned formulas, “—” is a single bond). The alkylene group in the “oxydialkylene group” is as explained above.

In the present specification, the “ester bond” means —CO—O— or —O—CO— (in the aforementioned formulas, “—” is a single bond).

In the present specification, the “amide bond” means —CO—NH— or —NH—CO— (in the aforementioned formulas, “—” is a single bond).

In the present specification, the “carbamate bond” means —O—CO—NH— or —NH—CO—O— (in the aforementioned formulas, “—” is a single bond).

In the present specification, the “ether bond” means —O— (in the aforementioned formulas, “—” is a single bond).

In the present specification, “urea bond” means —NH—CO—NH— (in the aforementioned formulas, “—” is a single bond).

In the present specification, “carbonate bond” means —O—CO—O— (in the aforementioned formulas, “—” is a single bond).

In the present specification, the “residue of a liposoluble vitamin having a hydroxy group” means a monovalent group having a structure obtained by removing a hydrogen atom from the hydroxy group of the aforementioned liposoluble vitamin. Examples of the liposoluble vitamin having a hydroxy group include retinol, ergosterol, 7-dehydrocholesterol, calciferol, cholecalciferol, dihydroergocalciferol, dihydrotachysterol, tocopherol, and tocotrienol.

In the present specification, the “sterol derivative having a hydroxy group residue” means a monovalent group having a structure obtained by removing a hydrogen atom from the hydroxy group of the aforementioned sterol derivative. Examples of the sterol derivative having a hydroxy group include cholesterol, cholestanol, stigmasterol, R-sitosterol, lanosterol, and ergosterol.

In the present specification, the alkyl group may be linear or branched. Examples of the alkyl group include methyl group, ethyl group, propyl group, isopropyl group, butyl group, isobutyl group, sec-butyl group, tert-butyl group, a pentyl group, isopentyl group, neopentyl group, tert-pentyl group, hexyl group, heptyl group, octyl group, nonyl group, decyl group, undecyl group, dodecyl group, tridecyl group, tetradecyl group, pentadecyl group, hexadecyl group, heptadecyl group, octadecyl group, nonadecyl group, icosyl group, henicosyl group, docosyl group, tricosyl group, tetracosyl group, pentacosyl group, hexacosyl group, heptacosyl group, octacosyl group, nonacosyl group, triacontyl group, hentriacontyl group, dotriacontyl group, tritriacontyl group, tetratriacontyl group, pentatriacontyl group, hexatriacontyl group, tetracontyl group, hentetracontyl group, dotetracontyl group, tritetracontyl group, and tetratetracontyl group.

In the present specification, the alkenyl group may be linear or branched. In the present specification, the number of the olefinic carbon-carbon double bond in the alkenyl group may be only one, or two or more. Examples of the alkenyl group include ethenyl group, propenyl group, butenyl group, pentenyl group, hexenyl group, heptenyl group, octenyl group, nonenyl group, decenyl group, undecenyl group, dodecenyl group, tridecenyl group, tetradecenyl group, pentadecenyl group, hexadecenyl group, heptadecenyl group, octadecenyl group, nonadecenyl group, icosenyl group, henicosenyl group, docosenyl group, tricosenyl group, tetracosenyl group, pentacosenyl group, hexacosenyl group, heptacosenyl group, octacosenyl group, nonacosenyl group, triacontenyl group, hentriacontenyl group, and dotriacontenyl group.

In the present specification, the alkynyl group may be linear or branched. In the present specification, the number of the carbon-carbon triple bond of the alkynyl group may be only one, or two or more. Examples of the alkynyl group include ethynyl group, propynyl group, butynyl group, pentynyl group, hexynyl group, heptynyl group, octynyl group, nonynyl group, decynyl group, undecynyl group, dodecynyl group, tridecynyl group, tetradecynyl group, pentadecynyl group, hexadecynyl group, heptadecynyl group, octadecynyl group, nonadecynyl group, icosynyl group, henicosynyl group, docosynyl group, tricosynyl group, tetracosinyl group, pentacosinyl group, hexacosinyl group, heptacosinyl group, octacosinyl group, nonacosinyl group, triacontinyl group, hentriacontinyl group, and dotriacontinyl group.

In the present specification, examples of the halogen atom include fluorine atom, chlorine atom, bromine atom, and iodine atom.

In the present specification, the “hydrocarbon ring group having 3 to 12 carbon atoms” means a cyclic group wherein the ring thereof is composed of 3 to 12 carbon atoms. Examples of the hydrocarbon ring group having 3 to 12 carbon atoms include cycloalkyl group having 3 to 8 carbon atoms, phenyl group, naphthyl group, and adamantyl group. Examples of the cycloalkyl group having 3 to 8 carbon atoms include cyclopropyl group, cyclobutyl group, cyclopentyl group, cyclohexyl group, cycloheptyl group, and cyclooctyl group. The hydrocarbon ring group having 3 to 12 carbon atoms is preferably a non-aromatic hydrocarbon ring group having 3 to 12 carbon atoms, more preferably a cycloalkyl group having 3 to 8 carbon atoms or an adamantyl group, further preferably a cyclohexyl group or an adamantyl group.

In the present specification, the “3- to 14-membered heterocyclic group” means a heterocyclic group wherein the ring thereof is composed of 3 to 14 carbon atoms. Other expressions similar to “3- to 14-membered” also have the same meaning as “3- to 14-membered”. Examples of the 3- to 14-membered heterocyclic group include 5 to 14-membered aromatic heterocyclic group, and 3- to 14-membered non-aromatic heterocyclic group.

In the present specification, examples of the 5 to 14-membered aromatic heterocyclic group include the following:
(i) 5 to 6-membered monocyclic aromatic heterocyclic groups such as thienyl group, furyl group, pyrrolyl group, imidazolyl group, pyrazolyl group, thiazolyl group, isothiazolyl group, oxazolyl group, isoxazolyl group, pyridyl group, pyrazinyl group, pyrimidinyl group, pyridazinyl group, 1,2,4-oxadiazolyl group, 1,3,4-oxadiazolyl group, 1,2,4-thiadiazolyl group, 1,3,4-thiadiazolyl group, triazolyl group, tetrazolyl group, triazinyl group, and the like;
(ii) 8 to 14-membered fused polycyclic aromatic heterocyclic groups such as benzothiophenyl group, benzofuranyl group, benzoimidazolyl group, benzoxazolyl group, benzoisoxazolyl group, benzothiazolyl group, benzoisothiazolyl group, benzotriazolyl group, imidazopyridinyl group, thienopyridinyl group, furopyridinyl group, pyrrolopyridinyl group, pyrazolopyridinyl group, oxazolopyridinyl group, thiazolopyridinyl group, imidazopyrazinyl group, imidazopyrimidinyl group, thienopyrimidinyl group, furopyrimidinyl group, pyrrolopyrimidinyl group, pyrazolopyrimidinyl group, oxazolopyrimidinyl group, thiazolopyrimidinyl group, pyrazolotriazinyl group, naphtho[2,3-b]thienyl group, phenoxathiinyl group, indolyl group, isoindolyl group, 1H-indazolyl group, purinyl group, isoquinolyl group, quinolyl group, phthalazinyl group, naphthyridinyl group, quinoxalinyl group, quinazolinyl group, cinnolinyl group, carbazolyl group, β-carbolinyl group, phenanthridinyl group, acrydinyl group, phenazinyl group, phenothiazinyl group, phenoxazinyl group, and the like.

In the present specification, examples of the 3- to 14-membered non-aromatic heterocyclic group include the following:
(i) 3 to 8-membered monocyclic non-aromatic heterocyclic groups such as aziridinyl group, oxiranyl group, thiiranyl group, azetidinyl group, oxetanyl group, thietanyl group, tetrahydrothienyl group, tetrahydrofuranyl group, pyrrolinyl group, pyrrolidinyl group, imidazolinyl group, imidazolidinyl so group, oxazolinyl group, oxazolidinyl group, pyrazolinyl group, pyrazolidinyl group, thiazolinyl group, thiazolidinyl group, tetrahydroisothiazolyl group, tetrahydroxazolyl group, tetrahydroisoxazolyl group, piperidinyl group, piperazinyl group, tetrahydropyridinyl group, dihydropyridinyl group, dihydrothiopyranyl group, tetrahydropyrimidinyl group, tetrahydropyridazinyl group, dihydropyranyl group, tetrahydropyranyl group, tetrahydrothiopyranyl group, morpholinyl group, thiomorpholinyl group, azepanyl group, diazepanyl group, azepinyl group, oxepanyl group, azocanyl group, diazocanyl group, and the like;
(ii) 9 to 14-membered fused polycyclic non-aromatic heterocyclic groups such as dihydrobenzofuranyl group, dihydrobenzoimidazolyl group, dihydrobenzoxazolyl group, dihydrobenzothiazolyl group, dihydrobenzoisothiazolyl group, dihydronaphto[2,3-b]thienyl group, tetrahydroisoquinolyl group, tetrahydroquinolyl group, 4H-quinolizinyl group, indolinyl group, isoindolinyl group, tetrahydrothieno[2,3-c]pyridinyl group, tetrahydrobenzoazepinyl group, tetrahydroquinoxalinyl group, tetrahydrophenanthridinyl group, hexahydrophenothiazinyl group, hexahydrophenoxazinyl group, tetrahydrophthalazinyl group, tetrahydronaphthyridinyl group, tetrahydroquinazolinyl group, tetrahydrocinnolinyl group, tetrahydrocarbazolyl group, tetrahydro-β-carbolinyl group, tetrahydroacrydinyl group, tetrahydrophenazinyl group, tetrahydrothioxanthenyl group, octahydroisoquinolyl group, and the like.

R^{1a}, R^{1b} and the like in the formula (1) are explained below. The explanations and preferred embodiments of the following R^{1a}, R^{1b} and the like can be combined with each other.

In the formula (1), R^{1a} and R^{1b} are each independently an alkylene group having 1 to 6 carbon atoms. R^{1a} and R^{1b} may be the same or different, and R^{1a} and R^{1b} are preferably the same groups. R^{1a} and R^{1b} are preferably each independently an alkylene group having 1 to 3 carbon atoms, more preferably each an alkylene group having 1 to 3 carbon atoms, further preferably an ethylene group. In the present specification, “R^{1a} and R^{1b} are each an alkylene group having 1 to 3 carbon atoms” means that R^{1a} and R^{1b} are the same and each is an alkylene group having 1 to 3 carbon atoms. Other expressions similar to “R^{1a} and R^{1b} are each an alkylene group having 1 to 3 carbon atoms” also have the same meaning as “R^{1a} and R^{1b} are each an alkylene group having 1 to 3 carbon atoms”.

In the formula (1), X^{a} and X^{b} are each independently an acyclic alkyl tertiary amino group having 1 to 6 carbon atoms and 1 tertiary amino group, or a cyclic alkylene tertiary amino group having 2 to 5 carbon atoms and 1 or 2 tertiary amino groups.

In the present specification, the “acyclic alkyl tertiary amino group having 1 to 6 carbon atoms and 1 tertiary amino group” means a divalent group represented by the formula (25):

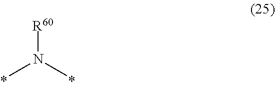

(in the formula (25), * is a bonding position, and
   R⁶⁰ is an alkyl group having 1 to 6 carbon atoms). In the present specification, “*”, etc. shows bonding positions, not carbon atoms, as described above.
   R⁶⁰ in the formula (25) is preferably a methyl group, an ethyl group, a propyl group, or an isopropyl group, more preferably a methyl group.

In the present specification, the “cyclic alkylene tertiary amino group having 2 to 5 carbon atoms and 1 or 2 tertiary amino groups” means a divalent group in which an alkylene group having 2 to 5 carbon atoms and a tertiary amino group form a cyclic structure, and 1 or 2 tertiary amino groups are contained in the aforementioned cyclic structure. The aforementioned carbon number is preferably 4 or 5.

Examples of the a cyclic alkylene tertiary amino group having 2 to 5 carbon atoms and 1 or 2 tertiary amino groups include aziridinediyl group, azetidinediyl group, pyrrolidinediyl group, piperidinediyl group, imidazolidinediyl group, and piperazinediyl group.

The cyclic alkylene tertiary amino group having 2 to 5 carbon atoms and 1 tertiary amino group is preferably a divalent group represented by the formula (26):

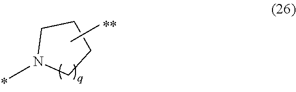

(in the formula (26), * is a bonding position with R^{1a} or R^{1b} in the formula (1),
   ** is a bonding position with R^{2a} or R^{2b} in the formula (1), and
   q is 1 or 2).

Hereinafter the “divalent group represented by the formula (26)” is sometimes to be referred to as “group (26)”. Groups represented by other formulas are also sometimes to be abbreviated in the same manner as the “divalent group represented by the formula (26)”. When q is 1, group (26) is a pyrrolidinediyl group, and when q is 2, group (26) is a piperidinediyl group.

A cyclic alkylene tertiary amino group having 2 to 5 carbon atoms and 2 tertiary amino groups is preferably a divalent group represented by the formula (27):

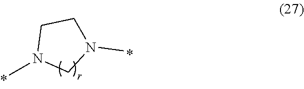

(in the formula (27), * is a bonding position, and
   r is 1 or 2). When r is is 1, group (27) is an imidazolidinediyl group. When r is 2, group (27) is a piperazinediyl group.
   X^{a} and X^{b} may be the same or different, and X^{a} and X^{b} are preferably the same groups. In the formula (1), X^{a} and X^{b} are preferably each independently a cyclic alkylene tertiary amino group having 2 to 5 carbon atoms and 1 or 2 tertiary amino groups, more preferably each independently group (26) or group (27), further preferably each independently group (26), particularly preferably each group (26) wherein q is 2 (i.e., piperidinediyl group).

In the formula (1), R^{2a} and R^{2b} are each independently an alkylene group having 1 to 8 carbon atoms or an oxydialkylene group having 2 to 8 carbon atoms. The oxydialkylene group having 2 to 8 carbon atoms is preferably an oxydimethylene group, an oxydiethylene group, an oxydipropylene group, more preferably an oxydiethylene group.

In the formula (1), R^{2a} and R^{2b} are preferably each independently an alkylene group having 1 to 8 carbon atoms, more preferably each independently an alkylene group having 1 to 4 carbon atoms, further preferably each an ethylene group.

In the formula (1), Y^{a} and Y^{b} are each independently an ester bond, an amide bond, a carbamate bond, an ether bond, or a urea bond. Y^{a} and Y^{b} may be the same or different, preferably Y^{a} and Y^{b} are the same bonds. Y^{a} and Y^{b} are preferably each independently an ester bond or an amide bond, more preferably each an ester bond, further preferably each *—CO—O—** (wherein * is a bonding position with Z^{a} or Z^{b} in the formula (1), and ** is a bonding position with R^{2a} or R^{2b} in the formula (1)).

In the formula (1), Z^{a} and Z^{b} are each independently a divalent group derived from an aromatic compound having 3 to 16 carbon atoms, at least one aromatic ring, and optionally having a heteroatom. In the following, the aforementioned aromatic ring and the aforementioned aromatic compound are sometimes to be abbreviated as “aromatic ring of Z^{a} and Z^{b}”.

The aromatic ring of Z^{a} and Z^{b} may be either an aromatic hydrocarbon ring or an aromatic heterocycle. Examples of the aromatic hydrocarbon ring include benzene ring, naphthalene ring, and anthracene ring. Examples of the aromatic heterocycle include imidazole ring, pyrazole ring, oxazole ring, isoxazole ring, thiazole ring, isothiazole ring, triazine ring, pyrrole ring, furanthiophene ring, pyrimidine ring, pyridazine ring, pyrazine ring, pyridine ring, purine ring, pteridine ring, benzimidazole ring, indole ring, benzofuran ring, quinazoline ring, phthalazine ring, quinoline ring, isoquinoline ring, coumarin ring, chromone ring, benzodiazepine ring, phenoxathiine ring, phenothiazine ring, and acridine ring. The aromatic ring of Z^{a} and Z^{b} is preferably an aromatic hydrocarbon ring, more preferably a benzene ring.

The aromatic ring of Z^{a} and Z^{b} may be substituted by a substituent. Examples of the substituent thereof include acyl group having 2 to 4 carbon atoms, alkoxycarbonyl group having 2 to 4 carbon atoms, alkylcarbamoyl group having 2 to 4 carbon atoms, acyloxy group having 2 to 4 carbon atoms, acylamino so group having 2 to 4 carbon atoms, alkoxycarbonylamino group having 2 to 4 carbon atoms, halogen atom (i.e., fluorine atom, chlorine atom, bromine atom, iodine atom), alkylsulfanyl group having 1 to 4 carbon atoms, alkylsulfonyl group having 1 to 4 carbon atoms, arylsulfonyl group having 6 to 10 carbon atoms, nitro group, trifluoromethyl group, cyano group, alkyl group having 1 to 4 carbon atoms, ureido group, alkoxy group having 1 to 4 carbon atoms, aryl group having 6 to 10 carbons, and aryloxy group having 6 to 10 carbon atoms. Preferred examples of the aforementioned substituent include acetyl group, methoxycarbonyl group, methylcarbamoyl group, acetoxy group, acetamido group, methoxycarbonylamino group, fluorine atom, chlorine atom, bromine atom, iodine atom, methylsulfanyl group, phenylsulfonyl group, nitro group, trifluoromethyl group, cyano group, methyl group, ethyl group, propyl group, isopropyl group, tert-butyl group, ureido group, methoxy group, ethoxy group, propoxy group, isopropoxy group, tert-butoxy group, phenyl group, and phenoxy group.

Z^{a} and Z^{b} are preferably each independently a divalent group represented by the formula (13):

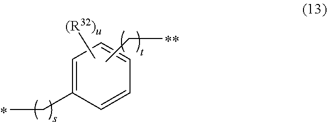

(in the formula (13), * is a bonding position with 0 in the formula (1),
   ** is a bonding position with Y^{a} or Y^{b} in the formula (1),
   s is an integer of 0 to 3,
   t is an integer of 0 to 3,
   u is an integer of 0 to 4, and
   R³² in the number of u are each independently a substituent). The aforementioned “s and t” shows the number of methylene groups (CH₂), and “s or t is is 0” means that the corresponding methylene group is not present. The aforementioned “u” shows the number of R³², and “u is is 0” means that R³² is not present.
   s is preferably 0 or 1, more preferably 0.
   t is preferably an integer of 0 to 2, more preferably 1.
   u is preferably an integer of 0 to 2, more preferably 0.
   R³² is
   preferably an acyl group having 2 to 4 carbon atoms, an alkoxycarbonyl group having 2 to 4 carbon atoms, an alkylcarbamoyl group having 2 to 4 carbon atoms, an acyloxy group having 2 to 4 carbon atoms, an acylamino group having 2 to 4 carbon atoms, an alkoxycarbonylamino group having 2 to 4 carbon atoms, a halogen atom (i.e., fluorine atom, chlorine atom, bromine atom, iodine atom), an alkylsulfanyl group having 1 to 4 carbon atoms, an alkylsulfonyl group having 1 to 4 carbon atoms, an arylsulfonyl group having 6 to 10 carbon atoms, a nitro group, a trifluoromethyl group, a cyano group, an alkyl group having 1 to 4 carbon atoms, a ureido group, an alkoxy group having 1 to 4 carbon atoms, an aryl group having 6 to 10 carbon atoms, or an aryloxy group having 6 to 10 carbon atoms,
   more preferably an acetyl group, a methoxycarbonyl group, a methylcarbamoyl group, an acetoxy group, an acetamido group, a methoxycarbonylamino group, a halogen atom (i.e., fluorine atom, chlorine atom, bromine atom, iodine atom), a methylsulfanyl group, a phenylsulfonyl group, a nitro group, a trifluoromethyl group, a cyano group, an alkyl group having 1 to 4 carbon atoms (e.g., methyl group, ethyl group, propyl group, isopropyl group, tert-butyl group), a ureido group, an alkoxy group having 1 to 4 carbon atoms (e.g., methoxy group, ethoxy group, propoxy group, isopropoxy group, tert-butoxy group), a phenyl group, or a phenoxy group.

When R³² is present in plurality, the R³² in plurality may be the same with or different from each other.

Z^{a} and Z^{b} may be the same or different, and Z^{a} and Z^{b} are preferably the same. Z^{a} and Z^{b} in the formula (1) are preferably each independently group (13), more preferably each independently group (13) in which s is 0 or 1, t is an integer of 0 to 2, and u is an integer of 0 to 2, more preferably each group (13) in which s is 0, t is 1, and u is 0.

R^{3a} in the formula (1) is a monovalent group recited below:
(ia) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group) (hereinafter sometimes to be abbreviated as “group (ia)”),
(iia) a monovalent group having 10 to 50 carbon atoms and at least two carbonyl groups (excluding a monovalent group containing a residue of a liposoluble vitamin having a hydroxy group and a residue of a sterol derivative having a hydroxy group) (hereinafter sometimes to be abbreviated as “group (iia)”),
(iiia) a monovalent group represented by the formula (2):

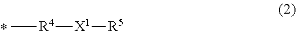

(in the formula (2), * is a bonding position,
   R⁴ is an alkylene group having 1 to 10 carbon atoms,
   X¹ is a carbamate bond, a carbonate bond, or an amide bond, and
   R⁵ is an alkyl group having 1 to 25 carbon atoms, and R⁵ is optionally substituted by a substituent selected from the group consisting of a halogen atom and a hydroxy group)
(hereinafter sometimes to be abbreviated as “group (iiia)”),
(iva) a monovalent group represented by the formula (3):

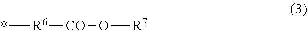

(in the formula (3), * is a bonding position,
   R⁶ is an alkylene group having 1 to 10 carbon atoms, and
   R⁷ is an alkyl group having 1 to 25 carbon atoms and substituted by at least one halogen atom)
(hereinafter sometimes to be abbreviated as “group (iva)”),
(va) a monovalent group represented by the formula (4):

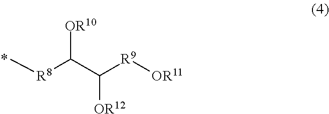

(in the formula (4), * is a bonding position,
   R⁸ and R⁹ are each independently an alkylene group having 1 to 10 carbon atoms, an alkenediyl group having 2 to 10 carbon atoms, or an alkynediyl group having 2 to 10 carbon atoms,
   R¹⁰ to R¹² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group))
(hereinafter sometimes to be abbreviated as “group (va)”),
(via) a monovalent group represented by the formula (5):

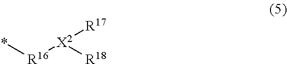

(in the formula (5), * is a bonding position,
   X² is a nitrogen atom or a trivalent group represented by the formula (6):

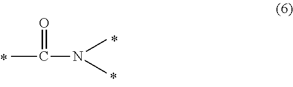

(in the formula (6), * is a bonding position with R¹⁶, and
   ** is a bonding position with R¹⁷ or R¹⁸),
   when X² is a nitrogen atom, R¹⁶ is an alkylene group having 1 to 10 carbon atoms, an alkenediyl group having 2 to 10 carbon atoms, or an alkynediyl group having 2 to 10 carbon atoms, and R¹⁶ is optionally substituted by a substituent selected from the group consisting of a halogen atom and a hydroxy group,
   when X² is a trivalent group represented by the formula (6), R¹⁶ is an alkylene group having 1 to 10 carbon atoms, and R¹⁶ is optionally substituted by a substituent selected from the group consisting of a halogen atom and a hydroxy group,
   when X² is a nitrogen atom, R¹⁷ and R¹⁸ are each independently an alkyl group having 1 to 10 carbon atoms, an alkenyl group having 2 to 10 carbon atoms, or an alkynyl group having 2 to 10 carbon atoms, and R¹⁷ and R¹⁸ are each independently optionally substituted by a substituent selected from the group consisting of a halogen atom and a hydroxy group, and
   when X² is a trivalent group represented by the formula (6), R¹⁷ and R¹⁸ are each independently an alkyl group having 1 to 10 carbon atoms, and R¹⁷ and R¹⁸ are each independently optionally substituted by a substituent selected from the group consisting of a halogen atom and a hydroxy group)
(hereinafter sometimes to be abbreviated as “group (via)”),
(viia) a monovalent group represented by the formula (7):

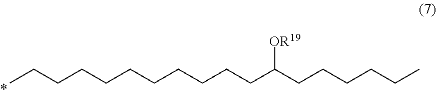

(in the formula (7), * is a bonding position, and
   R¹⁹ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group), or a *—CO—R²⁰ group (wherein * is a bonding position, and R²⁰ is an alkyl group having 1 to 9 carbon atoms))
(hereinafter sometimes to be abbreviated as “group (viia)”),
(viiia) a monovalent group represented by the formula (8):

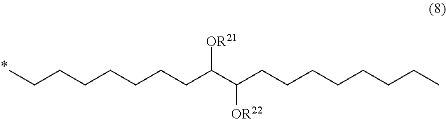

(in the formula (8), * is a bonding position, and
   R²¹ and R²² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group))
(hereinafter sometimes to be abbreviated as “group (viiia)”),
(ixa) a monovalent group represented by the formula (9):

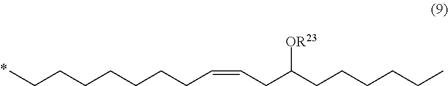

(in the formula (9), * is a bonding position, and
   R²³ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group))
(hereinafter sometimes to be abbreviated as “group (ixa)”),
(xa) a monovalent group represented by the formula (10):

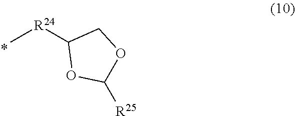

(in the formula (10), * is a bonding position,
   R²⁴ is an alkylene group having 1 to 10 carbon atoms, an alkenediyl group having 2 to 10 carbon atoms, or an alkynediyl group having 2 to 10 carbon atoms, and
   R²⁵ is an alkyl group having 1 to 30 carbon atoms, an alkenyl group having 2 to 30 carbon atoms, or an alkynyl group having 2 to 30 carbon atoms)
(hereinafter sometimes to be abbreviated as “group (xa)”),
(xia) a monovalent group represented by the formula (11):

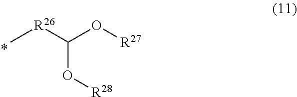

(in the formula (11), * is a bonding position, and
   R²⁶ is an alkylene group having 1 to 10 carbon atoms, an alkenediyl group having 2 to 10 carbon atoms, or an alkynediyl group having 2 to 10 carbon atoms, and
   R²⁷ and R²⁸ are each independently an alkyl group having 1 to 10 carbon atoms, an alkenyl having 2 to 10 carbon atoms, or an alkynyl group having 2 to 10 carbon atoms)
(hereinafter sometimes to be abbreviated as “group (xia)”), or
(xiia) a monovalent group represented by the formula (12):

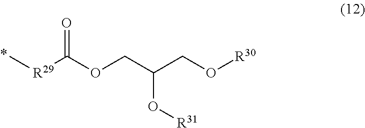

(in the formula (12), * is a bonding position,
   R²⁹ is an alkylene group having 1 to 10 carbon atoms, and
   R³⁰ and R³¹ are each independently an alkyl group having 1 to 10 carbon atoms)
(hereinafter sometimes to be abbreviated as “group (xiia)”).

R^{3b} in the formula (1) is a monovalent group recited below:
(ib) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group) (hereinafter sometimes to be abbreviated as “group (ib)”),
(iib) a monovalent group having 10 to 50 carbon atoms and at least two carbonyl groups (excluding a monovalent group containing a residue of a liposoluble vitamin having a hydroxy group and a residue of a sterol derivative having a hydroxy group) (hereinafter sometimes to be abbreviated as “group (iib)”),
(iiib) a monovalent group represented by the aforementioned formula (2) (hereinafter sometimes to be abbreviated as “group (iiib)”)
(ivb) a monovalent group represented by the aforementioned formula (3) (hereinafter sometimes to be abbreviated as “group (ivb)”),
(vb) a monovalent group represented by the aforementioned formula (4) (hereinafter sometimes to be abbreviated as “group (vb)”),
(vib) a monovalent group represented by the aforementioned formula (5) (hereinafter sometimes to be abbreviated as “group (vib)”),
(viib) a monovalent group represented by the aforementioned formula (7) (hereinafter sometimes to be abbreviated as “group (viib)”),
(viiib) a monovalent group represented by the aforementioned formula (8) (hereinafter sometimes to be abbreviated as “group (viiib)”)
(ixb) a monovalent group represented by the aforementioned formula (9) (hereinafter sometimes to be abbreviated as “group (ixb)”),
(xb) a monovalent group represented by the aforementioned formula (10) (hereinafter sometimes to be abbreviated as “group (xb)”),
(xib) a monovalent group represented by the aforementioned formula (11) (hereinafter sometimes to be abbreviated as “group (xib)”),
(xiib) a monovalent group represented by the aforementioned formula (12) (hereinafter sometimes to be abbreviated as “group (xiib)”),
(xiiib) a monovalent group which is an alkyl group having 1 to 30 carbon atoms, an alkenyl group having 2 to 20 carbon atoms, or an alkynyl group having 2 to 20 carbon atoms, in which one ethylene group in the aforementioned alkyl group is optionally replaced by one ester bond (hereinafter sometimes to be abbreviated as “group (xiiib)”), or
(xivb) a R^{3c}—CO—(CH₂)ₚ— group (wherein R^{3c} is a residue of a liposoluble vitamin having a hydroxy group or a residue of a sterol derivative having a hydroxy group, and p is an integer of 1 to 8) (hereinafter sometimes to be abbreviated as “group (xivb)”).

In the formula (1), R^{3a} and R^{3b} may be the same or different. In the following, “group (ia) and group (ib)” and the like may be collectively referred to as “group (i)” and the like.

In a preferred embodiment of the present invention, one or both of R^{3a} and R^{3b} are group (i) or group (ii). In other words, in a preferred embodiment of the present invention, R^{3a} is group (ia) or group (iia), and R^{3b} is any of group (ib) to group (xivb). In the aforementioned embodiment, R^{3b} is preferably group (ib), group (iib), group (xiiib), or group (xivb). In the aforementioned embodiment, R^{3a} and R^{3b} may be the same or different.

In another embodiment of the present invention, R^{3a} is any of group (iiia) to group (xiia), and R^{3b} is any of group (iiib) to group (xivb). In the aforementioned embodiment, R^{3b} is preferably group (xiiib) or group (xivb), more preferably group (xiiib). In the aforementioned embodiment, R^{3a} and R^{3b} may be the same or different.

R^{3a} is preferably a monovalent group recited below:
(ia-1) a monovalent group having 10 to 50 carbon atoms and represented by the formula (14):

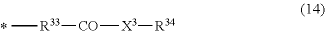

(in the formula (14), * is a bonding position,
   R³³ is an alkylene group having 2 to 10 carbon atoms, an alkenediyl group having 2 to 10 carbon atoms, or an alkynediyl group having 2 to 10 carbon atoms, and R³³ is optionally substituted by a substituent selected from the group consisting of a halogen atom, a hydroxy group, and a hydrocarbon ring group having 3 to 12 carbon atoms,
   R³⁴ is an alkyl group having 1 to 40 carbon atoms, an alkenyl group having 2 to 40 carbon atoms, or an alkynyl group having 2 to 40 carbon atoms, and R³⁴ is optionally substituted by a substituent selected from the group consisting of a halogen atom, a hydroxy group, and a hydrocarbon ring group having 3 to 12 carbon atoms,
   at least one of R³³ and R³⁴ has at least one unsaturated bond selected from the group consisting of an olefinic carbon-carbon double bond and a carbon-carbon triple bond, and
   X³ is an oxygen atom, NH, or a sulfur atom)
(hereinafter sometimes to be abbreviated as “group (ia-1)”),
(ia-2) a monovalent group having 50 or less carbon atoms and represented by the formula (15):

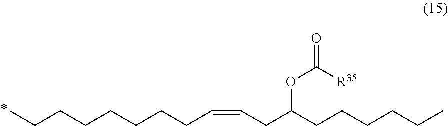

(in the formula (15), * is a bonding position, and
   R³⁵ is an alkyl group having 1 to 20 carbon atoms, an alkenyl group having 2 to 20 carbon atoms, an alkynyl group having 2 to 20 carbon atoms, or a hydrocarbon ring group having 3 to 12 carbon atoms, and R³⁵ is optionally substituted by a substituent selected from the group consisting of a 3- to 14-membered heterocyclic group, and a hydrocarbon ring group having 3 to 12 carbon atoms)
(hereinafter sometimes to be abbreviated as “group (ia-2)”),
(iia-1) a monovalent group having 50 or less carbon atoms and represented by the formula (16):

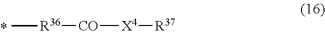

(in the formula (16), * is a bonding position,
   R³⁶ is an alkylene group having 2 to 9 carbon atoms, an alkenediyl group having 2 to 9 carbon atoms, or an alkynediyl group having 2 to 9 carbon atoms, and R³⁶ is optionally substituted by a substituent selected from the group consisting of a halogen atom, a hydroxy group, and a hydrocarbon ring group having 3 to 12 carbon atoms,
   R³⁷ is an alkyl group having 7 to 45 carbon atoms, an alkenyl group having 7 to 45 carbon atoms, or an alkynyl group having 7 to 45 carbon atoms, at least one ethylene group or at least one trimethylene group in R³⁷ is replaced by at least one bond selected from the group consisting of an ester bond, an amide bond, a carbamate bond, and a carbonate bond, and R³⁷ is optionally substituted by a substituent selected from the group consisting of a halogen atom, a hydroxy group, and a hydrocarbon ring group having 3 to 12 carbon atoms, and
   X⁴ is an oxygen atom, NH, or a sulfur atom)
(hereinafter sometimes to be abbreviated as “group (iia-1)”),
(iia-2) a monovalent group having 10 to 50 carbon atoms and represented by the formula (17):

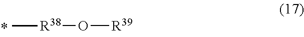

(in the formula (17), * is a bonding position, and
   R³⁸ is an alkylene group having 2 to 10 carbon atoms, an alkenediyl group having 2 to 10 carbon atoms, or an alkynediyl group having 2 to 10 carbon atoms, at least one ethylene group or at least one trimethylene group in R³⁸ is optionally replaced by at least one bond selected from the group consisting of an ester bond, an amide bond, a carbamate bond, and a carbonate bond, and R³⁸ is optionally substituted by a substituent selected from the group consisting of a halogen atom, a hydroxy group, and a hydrocarbon ring group having 3 to 12 carbon atoms, and
   R³⁹ is an alkyl group having 3 to 30 carbon atoms, an alkenyl group having 4 to 30 carbon atoms, or an alkynyl group having 4 to 30 carbon atoms, at least two methylene groups in R³⁹ are replaced by at least two carbonyl groups, and at least one methylene group of R³⁹ is optionally replaced by at least one ether bond)
(hereinafter sometimes to be abbreviated as “group (iia-2)”),
(iia-3) a monovalent group having 50 or less carbon atoms and represented by the formula (18):

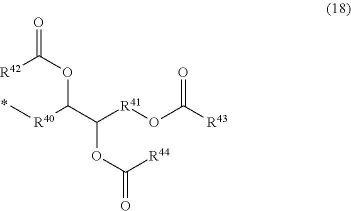

(in the formula (18), * is a bonding position,
   R⁴⁰ and R⁴¹ are each independently an alkylene group having 3 to 10 carbon atoms, and
   R⁴² to R⁴⁴ are each independently an alkyl group having 1 to 10 carbon atoms, an alkenyl group having 2 to 10 carbon atoms or an alkynyl group having 2 to 10 carbon atoms, at least one ethylene group or at least one trimethylene group in R⁴² is optionally replaced by at least one bond selected from the group consisting of an ester bond, an amide bond, a carbamate bond, and a carbonate bond, at least one ethylene group or at least one trimethylene group in R⁴³ is optionally replaced by at least one bond selected from the group consisting of an ester bond, an amide bond, a carbamate bond, and a carbonate bond, at least one ethylene group or at least one trimethylene group in R⁴⁴ is optionally replaced by at least one bond selected from the group consisting of an ester bond, an amide bond, a carbamate bond, and a carbonate bond, and R⁴² to R⁴⁴ are each independently optionally substituted by a substituent selected from the group consisting of a halogen atom, a hydroxy group, and a hydrocarbon ring group having 3 to 12 carbon atoms)
(hereinafter sometimes to be abbreviated as “group (iia-3)”), or
(iia-4) a monovalent group having 50 or less carbon atoms and represented by the formula (19):

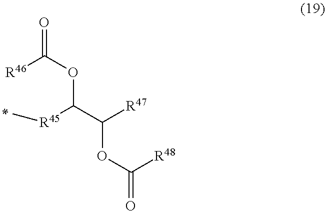

(in the formula (19), * is a bonding position,
   R⁴⁵ is an alkylene group having 5 to 10 carbon atoms, and
   R⁴⁶ to R⁴⁸ are each independently an alkyl group having 1 to 10 carbon atoms, an alkenyl group having 2 to 10 carbon atoms, or an alkynyl group having 2 to 10 carbon atoms, at least one ethylene group or at least one trimethylene group in R⁴⁶ is optionally replaced by at least one bond selected from the group consisting of an ester bond, an amide bond, a carbamate bond, and a carbonate bond, at least one ethylene group or at least one trimethylene group in R⁴⁷ is optionally replaced by at least one bond selected from the group consisting of an ester bond, an amide bond, a carbamate bond, and a carbonate bond, at least one ethylene group or at least one trimethylene group in R⁴⁸ is optionally replaced by at least one bond selected from the group consisting of an ester bond, an amide bond, a carbamate bond, and a carbonate bond, and R⁴⁶ to R⁴⁸ are each independently optionally substituted by a substituent selected from the group consisting of a halogen atom, a hydroxy group, and a hydrocarbon ring group having 3 to 12 carbon atoms)
(hereinafter sometimes to be abbreviated as “group (iia-4)”).
   R^{3b} is preferably a monovalent group recited below:
(ib-1) a monovalent group having 10 to 50 carbon atoms and represented by the aforementioned formula (14) (hereinafter sometimes to be abbreviated as “group (ib-1)”),
(ib-2) a monovalent group having 50 or less carbon atoms and represented by the aforementioned formula (15) (hereinafter sometimes to be abbreviated as “group (ib-2)”),
(iib-1) a monovalent group having 50 or less carbon atoms and represented by the aforementioned formula (16) (hereinafter sometimes to be abbreviated as “group (iib-1)”),
(iib-2) a monovalent group having 10 to 50 carbon atoms and represented by the aforementioned formula (17) (hereinafter sometimes to be abbreviated as “group (iib-2)”),
(iib-3) a monovalent group having 50 or less carbon atoms and represented by the aforementioned formula (18) (hereinafter sometimes to be abbreviated as “group (iib-3)”),
(iib-4) a monovalent group having 50 or less carbon atoms and represented by the aforementioned formula (19) (hereinafter sometimes to be abbreviated as “group (iib-4)”), or any of group (iiib) to group (xivb).

Group (ia) and the like are explained below in order.

Group (ia) is preferably group (ia-1) (i.e., group (14)) or group (ia-2) (i.e., group (15)). Group (ib) is preferably group (ib-1) (i.e., group (14)) or group (ib-2) (i.e., group (15)).

R³³ in the formula (14) is as mentioned above, and an alkylene group having 2 to 10 carbon atoms, an alkenediyl group having 2 to 10 carbon atoms, or an alkynediyl group having 2 to 10 carbon atoms, and R³³ is optionally substituted by a substituent selected from the group consisting of a halogen atom, a hydroxy group, and a hydrocarbon ring group having 3 to 12 carbon atoms.

In the present specification, “R³³ is optionally substituted by a substituent selected from the group consisting of a halogen atom, a hydroxy group, and a hydrocarbon ring group having 3 to 12 carbon atoms” (hereinafter abbreviated as “the aforementioned expression”) means that the aforementioned alkylene group, the aforementioned alkenediyl group and the aforementioned alkynediyl group for R³³ are each independently optionally substituted by a substituent selected from the group consisting of a halogen atom, a hydroxy group, and a hydrocarbon ring group having 3 to 12 carbon atoms. Other expressions similar to the aforementioned expression have the same meaning as the aforementioned expression.

R³³ in the formula (14) is preferably an alkylene group having 2 to 10 carbon atoms, an alkenediyl group having 2 to 10 carbon atoms, or an alkynediyl group having 2 to 10 carbon atoms, more preferably an alkylene group having 2 to 8 carbon atoms or an alkenediyl group having 2 to 8 carbon atoms. In the present specification, unless otherwise specified regarding the substituent, “alkylene group”, “alkenediyl group”, “alkynediyl group”, “alkyl group”, “alkenyl group”, and “alkynyl group” show unsubstituted groups.

R³⁴ in the formula (14) is preferably an alkyl group having 1 to 40 carbon atoms, an alkenyl group having 2 to 40 carbon atoms, or an alkynyl group having 2 to 40 carbon atoms, more preferably an alkyl group having 1 to 20 carbon atoms, an alkenyl group having 2 to 20 carbon atoms, or an alkynyl group having 2 to 20 carbon atoms.

At least one of R³³ and R³⁴ in the formula (14) has, as mentioned above, at least one unsaturated bond selected from the group consisting of an olefinic carbon-carbon double bond and a carbon-carbon triple bond.

X³ in the formula (14) is preferably an oxygen atom.

Group (14) is preferably a monovalent group derived from intermediate 19, intermediate 20, intermediate 21, or intermediate 22 mentioned below. In the present specification, the “monovalent group derived from intermediate X” (X: integer of one or more) means a monovalent group having a structure obtained by removing a carboxy group from intermediate X.

R³⁵ in the formula (15) is preferably an alkyl group having 1 to 20 carbon atoms, an alkenyl group having 2 to 20 carbon atoms, an alkynyl group having 2 to 20 carbon atoms, or a hydrocarbon ring group having 3 to 12 carbon atoms, and the aforementioned alkyl group is optionally substituted by a hydrocarbon ring group having 3 to 12 carbon atoms.

R³⁵ in the formula (15) is more preferably an alkyl group having 1 to 20 carbon atoms, an alkenyl group having 2 to 20 carbon atoms, an alkynyl group having 2 to 20 carbon atoms, or a cyclohexyl group, and the aforementioned alkyl group is optionally substituted by an adamantyl group.

In one embodiment of the present invention, R³⁵ in the formula (15) is further preferably an alkyl group having 1 to 20 carbon atoms, an alkenyl group having 2 to 20 carbon atoms, or an alkynyl group having 2 to 20 carbon atoms, and the aforementioned alkyl group is optionally substituted by a cyclohexyl group.

R³⁵ in the formula (15) is particularly preferably an alkyl group having 1 to 20 carbon atoms, an alkenyl group having 2 to 20 carbon atoms, or an alkynyl group having 2 to 20 carbon atoms.

Group (15) is preferably a monovalent group derived from intermediate 2, intermediate 3, intermediate 4, intermediate 5, intermediate 6, intermediate 7, intermediate 8, intermediate 9, intermediate 10, intermediate 55, intermediate 61, intermediate 62, intermediate 63, intermediate 64, intermediate 65, or intermediate 66 mentioned below, more preferably a monovalent group derived from intermediate 2, intermediate 3, intermediate 4, intermediate 5, intermediate 6, intermediate 7, intermediate 8, intermediate 9, intermediate 10, or intermediate 55.

Group (iia) is preferably group (iia-1) (i.e., group (16)), group (iia-2) (i.e., group (17)), group (iia-3) (i.e., group (18)), or group (iia-4) (i.e., group (19)). Group (iib) is preferably group (iib-1) (i.e., group (16)), group (iib-2) (i.e., group (17)), group (iib-3) (i.e., group (18)), or group (iib-4) (i.e., group (19)).

X⁴ in the formula (16) is preferably an oxygen atom or NH, more preferably an oxygen atom.

R³⁶ in the formula (16) is preferably an alkylene group having 2 to 9 carbon atoms, an alkenediyl group having 2 to 9 carbon atoms, or an alkynediyl group having 2 to 9 carbon atoms, more preferably an alkylene group having 2 to 9 carbon atoms or an alkenediyl group having 2 to 9 carbon atoms, further preferably an alkylene group having 2 or 3 carbon atoms or an alkenediyl group having 2 or 3 carbon atoms.

R³⁶ in the formula (16) is preferably an alkylene group having 2 to 9 carbon atoms, an alkenediyl group having 2 to 9 carbon atoms, or an alkynediyl group having 2 to 9 carbon atoms, more preferably an alkylene group having 2 to 9 carbon atoms, further preferably an alkylene group having 2 or 3 carbon atoms.

R³⁷ in the formula (16) is, as mentioned above, an alkyl group having 7 to 45 carbon atoms, an alkenyl group having 7 to 45 carbon atoms, or an alkynyl group having 7 to 45 carbon atoms, at least one ethylene group or at least one trimethylene group in R³⁷ is replaced by at least one bond selected from the group consisting of an ester bond, an amide bond, a carbamate bond, and a carbonate bond, and R³⁷ is optionally substituted by a substituent selected from the group consisting of a halogen atom, a hydroxy group, and a hydrocarbon ring group having 3 to 12 carbon atoms.

In the present specification, “at least one ethylene group or at least one trimethylene group in R³⁷ is replaced by at least one bond selected from the group consisting of an ester bond, an amide bond, a carbamate bond, and a carbonate bond” (hereinafter abbreviated as “the aforementioned expression”) means that at least one ethylene group or at least one trimethylene group in the aforementioned alkyl group for R³⁷ is replaced by at least one bond selected from the group consisting of an ester bond, an amide bond, a carbamate bond, and a carbonate bond, or at least one ethylene group or at least one trimethylene group in the aforementioned alkenyl for R³⁷ is replaced by at least one bond selected from the group consisting of an ester bond, an amide bond, a carbamate bond, and a carbonate bond, or at least one ethylene group or at least one trimethylene group in the aforementioned alkynyl group for R³⁷ is replaced by at least one bond selected from the group consisting of an ester bond, an amide bond, a carbamate bond, and a carbonate bond. Other expressions similar to the aforementioned expression have the same meaning as the aforementioned expression.

R³⁷ in the formula (16) is preferably
(iia-1-1) a monovalent group represented by the formula (20):

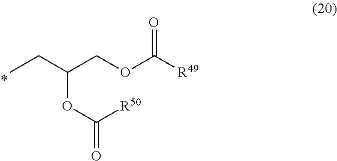

(in the formula (20), * is a bonding position, and
   R⁴⁹ and R⁵⁰ are each independently an alkyl group having 1 to 17 carbon atoms, an alkenyl group having 2 to 17 carbon atoms, or an alkynyl group having 2 to 17 carbon atoms, at least one ethylene group or at least one trimethylene group in R⁴⁹ is optionally replaced by at least one bond selected from the group consisting of an ester bond, an amide bond, a carbamate bond, and a carbonate bond, at least one ethylene group or at least one trimethylene group in R⁵⁰ is optionally replaced by at least one bond selected from the group consisting of an ester bond, an amide bond, a carbamate bond, and a carbonate bond, and R⁴⁹ and R⁵⁰ are each independently optionally substituted by a substituent selected from the group consisting of a halogen atom, a hydroxy group, and a hydrocarbon ring group having 3 to 12 carbon atoms),
(iia-1-2) a monovalent group represented by the formula (21):

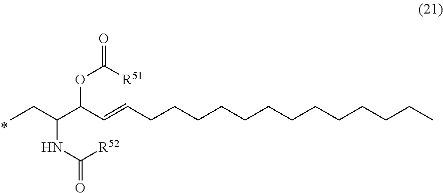

(in the formula (21), * is a bonding position, and
   R⁵¹ and R⁵² are each independently an alkyl group having 1 to 10 carbon atoms, an alkenyl group having 2 to 10 carbon atoms, or an alkynyl group having 2 to 10 carbon atoms, at least one ethylene group or at least one trimethylene group in R⁵¹ is optionally replaced by at least one bond selected from the group consisting of an ester bond, an amide bond, a carbamate bond, and a carbonate bond, at least one ethylene group or at least one trimethylene group in R⁵² is optionally replaced by at least one bond selected from the group consisting of an ester bond, an amide bond, a carbamate bond, and a carbonate bond, and R⁵¹ and R⁵² are each independently optionally substituted by a substituent selected from the group consisting of a halogen atom, a hydroxy group, and a hydrocarbon ring group having 3 to 12 carbon atoms), or
(iia-1-3) a monovalent group represented by the formula (22):

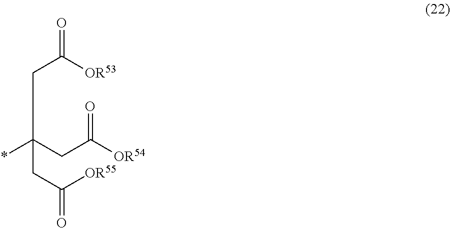

(in the formula (22), * is a bonding position, and
   R⁵³ to R⁵⁵ are each independently an alkyl group having 1 to 10 carbon atoms, an alkenyl group having 2 to 10 carbon atoms, or an alkynyl group having 2 to 10 carbon atoms, at least one ethylene group or at least one trimethylene group in R⁵³ is optionally replaced by at least one bond selected from the group consisting of an ester bond, an amide bond, a carbamate bond, and a carbonate bond, at least one ethylene group or at least one trimethylene group in R⁵⁴ is optionally replaced by at least one bond selected from the group consisting of an ester bond, an amide bond, a carbamate bond, and a carbonate bond, at least one ethylene group or at least one trimethylene group in R⁵⁵ is optionally replaced by at least one bond selected from the group consisting of an ester bond, an amide bond, a carbamate bond, and a carbonate bond, and R⁵³ to R⁵⁵ are each independently optionally substituted by a substituent selected from the group consisting of a halogen atom, a hydroxy group, and a hydrocarbon ring group having 3 to 12 carbon atoms).

In other words, group (16) is preferably a monovalent group having 50 or less carbon atoms and represented by the following formula (16-20), a monovalent group having 50 or less carbon atoms and represented by the following formula (16-21), or a monovalent group having 50 or less carbon atoms and represented by the following formula (16-22) (the definitions of the symbols in the following formulas are as mentioned above).

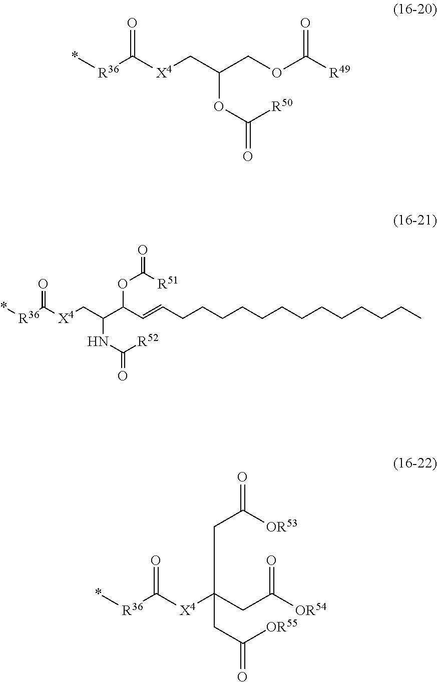

The explanation of R³⁶ and X⁴ in the formula (16-20) to the formula (16-22) is as mentioned above.

R⁴⁹ and R⁵⁰ in the formula (20) and the formula (16-20) are preferably each independently an alkyl group having 1 to 17 carbon atoms, an alkenyl group having 2 to 17 carbon atoms, or an alkynyl group having 2 to 17 carbon atoms.

R⁵¹ and R⁵² in the formula (21) and the formula (16-21) are preferably each independently an alkyl group having 1 to 10 carbon atoms, an alkenyl group having 2 to 10 carbon atoms, or an alkynyl group having 2 to 10 carbon atoms, more preferably each independently an alkyl group having 1 to 10 carbon atoms.

R⁵³ to R⁵⁵ in the formula (22) and the formula (16-22) are preferably each independently an alkyl group having 1 to 10 carbon atoms, an alkenyl group having 2 to 10 carbon atoms, or an alkynyl group having 2 to 10 carbon atoms, more preferably an alkyl group having 1 to 10 carbon atoms.

Group (16-20) is preferably a monovalent group derived from intermediate 11, intermediate 12, intermediate 13, intermediate 14, intermediate 15, intermediate 16, intermediate 26, intermediate 27, intermediate 28, intermediate 29, or intermediate 59 mentioned below.

Group (16-21) is preferably a monovalent group derived from intermediate 23 mentioned below.

Group (16-22) is preferably a monovalent group derived from intermediate 24 or intermediate 25 mentioned below.

R³⁸ in the formula (17) is preferably an alkylene group having 2 to 10 carbon atoms, an alkenediyl group having 2 to 10 carbon atoms, or an alkynediyl group having 2 to 10 carbon atoms, more preferably an alkylene group having 2 to 10 carbon atoms, further preferably an alkylene group having 2 or 3 carbon atoms.

R³⁹ in the formula (17) is preferably
(iia-2-1) a monovalent group represented by the formula (23):

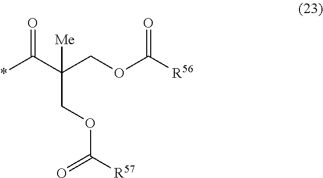

(in the formula (23), * is a bonding position,
   Me is a methyl group, and
   R⁵⁶ and R⁵⁷ are each independently an alkyl group having 1 to 10 carbon atoms, an alkenyl group having 2 to 10 carbon atoms, or an alkynyl group having 2 to 10 carbon atoms, at least one ethylene group or at least one trimethylene group in R⁵⁶ is optionally replaced by at least one bond selected from the group consisting of an ester bond, an amide bond, a carbamate bond, and a carbonate bond, at least one ethylene group or at least one trimethylene group in R⁵⁷ is optionally replaced by at least one bond selected from the group consisting of an ester bond, an amide bond, a carbamate bond, and a carbonate bond, and R⁵⁶ and R⁵⁷ are each independently optionally substituted by a substituent selected from the group consisting of a halogen atom, a hydroxy group, and a hydrocarbon ring group having 3 to 12 carbon atoms), or
(iia-2-2) a monovalent group represented by the formula (24):

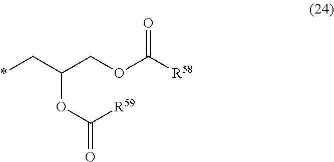

(in the formula (24), * is a bonding position, and
   R⁵⁸ and R⁵⁹ are each independently an alkyl group having 1 to 17 carbon atoms, an alkenyl group having 2 to 17 carbon atoms, or an alkynyl group having 2 to 17 carbon atoms, at least one ethylene group or at least one trimethylene group in R⁵⁸ is optionally replaced by at least one bond selected from the group consisting of an ester bond, an amide bond, a carbamate bond, and a carbonate bond, at least one ethylene group or at least one trimethylene group in R⁵⁹ is optionally replaced by at least one bond selected from the group consisting of an ester bond, an amide bond, a carbamate bond, and a carbonate bond, and R⁵⁸ and R⁵⁹ are each independently optionally substituted by a substituent selected from the group consisting of a halogen atom, a hydroxy group, and a hydrocarbon ring group having 3 to 12 carbon atoms).

In other words, group (17) is preferably a monovalent group having 50 or less carbon atoms and represented by the following formula (17-23), or a monovalent group having 1 to 50 carbon atoms and represented by the following formula (17-24) (the definitions of the symbols in the following formulas are as mentioned above).

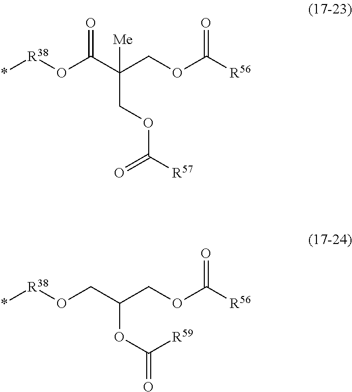

The explanation of R³⁸ in the formula (17-23) and the formula (17-24) is as mentioned above.

Me in the formula (17-23) is, as mentioned above, a methyl group.

R⁵⁶ and R⁵⁷ in the formula (23) and the formula (17-23) are preferably each independently an alkyl group having 1 to 10 carbon atoms, an alkenyl group having 2 to 10 carbon atoms, or an alkynyl group having 2 to 10 carbon atoms, more preferably each independently an alkyl group having 1 to 10 carbon atoms.

R⁵⁸ and R⁵⁹ in the formula (24) and the formula (17-24) are preferably each independently an alkyl group having 1 to 17 carbon atoms, an alkenyl group having 2 to 17 carbon atoms, or an alkynyl group having 2 to 17 carbon atoms, more preferably each independently an alkyl group having 1 to 17 carbon atoms, further preferably each independently an alkyl group having 1 to 10 carbon atoms.

Group (17-23) is preferably a monovalent group derived from intermediate 32 or intermediate 33 mentioned below.

Group (17-24) is preferably a monovalent group derived from intermediate 34 or intermediate 35 mentioned below.

R⁴⁰ and R⁴¹ in the formula (18) are, as mentioned above, each independently an alkylene group having 3 to 10 carbon atoms.

R⁴² to R⁴⁴ in the formula (18) are preferably each independently an alkyl group having 1 to 10 carbon atoms, an alkenyl group having 2 to 10 carbon atoms, or an alkynyl group having 2 to 10 carbon atoms, more preferably an alkyl group having 1 to 10 carbon atoms.

Group (18) is preferably a monovalent group derived from intermediate 36 mentioned below.

R⁴⁵ in the formula (19) is, as mentioned above, an alkylene group having 5 to 10 carbon atoms.

R⁴⁶ to R⁴⁸ in the formula (19) are preferably each independently an alkyl group having 1 to 10 carbon atoms, an alkenyl group having 2 to 10 carbon atoms, or an alkynyl group having 2 to 10 carbon atoms, more preferably each independently an alkyl group having 1 to 10 carbon atoms.

Group (19) is preferably a monovalent group derived from intermediate 17 or intermediate 18 mentioned below.

R⁴ in the formula (2) is, as mentioned above, an alkylene group having 1 to 10 carbon atoms.

X¹ in the formula (2) is preferably *—NH—CO—O—** (wherein * is a bonding position with R⁴ in the formula (2), and ** is a bonding position with R⁵ in the formula (2)), or *—O—CO—O—* (wherein * is a bonding position).

Group (2) is preferably a monovalent group derived from intermediate 40 or intermediate 41 mentioned below.

R⁶ in the formula (3) is, as mentioned above, an alkylene group having 1 to 10 carbon atoms.

R⁷ in the formula (3) is preferably an alkyl group having 1 to 25 carbon atoms substituted by at least one fluorine atom.

Group (3) is preferably a monovalent group derived from intermediate 39 mentioned below.

R⁸ and R⁹ in the formula (4) are preferably each independently an alkylene group having 1 to 10 carbon atoms.

R¹⁰ to R¹² in the formula (4) are preferably each independently a hydrogen atom, a benzyl group, or a *—Si(R¹³)(R¹⁴)(R¹⁵) group (wherein * is a bonding position, R¹³ is a tert-butyl group, and R¹⁴ and R¹⁵ are each a methyl group or a phenyl group), more preferably each independently a hydrogen atom or a *—Si(R¹³)(R¹⁴)(R¹⁵) group (wherein * is a bonding position, R¹³ is a tert-butyl group, and R¹⁴ and R¹⁵ are each a methyl group or a phenyl group), further preferably each independently a hydrogen atom or a *—Si(R¹³)(R¹⁴)(R¹⁵) group (wherein * is a bonding position, R¹³ is a tert-butyl group, and R¹⁴ and R¹⁵ are each a methyl group).

Group (4) is preferably a monovalent group derived from intermediate 42 mentioned below, or a monovalent group derived from deprotected intermediate 42 (i.e., compound in which the tert-butyldimethylsilyloxy group of intermediate 42 is replaced by a hydroxy group).

X² in the formula (5) is, as mentioned above, a nitrogen atom or group (6).

When X² in the formula (5) is a nitrogen atom, R¹⁶ in the formula (5) is preferably an alkylene group having 1 to 10 carbon atoms, an alkenediyl group having 2 to 10 carbon atoms, or an alkynediyl group having 2 to 10 carbon atoms, more preferably an alkylene group having 1 to 10 carbon atoms.

When X² in the formula (5) is group (6), R¹⁶ in the formula (5) is preferably an alkylene group having 1 to 10 carbon atoms.

When X² in the formula (5) is a nitrogen atom or group (6), R¹⁷ and R¹⁸ in the formula (5) are preferably each independently an alkyl group having 1 to 10 carbon atoms, and R¹⁷ and R¹⁸ are each independently optionally substituted by a hydroxy group.

Group (5) is preferably a monovalent group derived from intermediate 37, intermediate 38, intermediate 43, intermediate 44, or intermediate 47 mentioned below.

R¹⁹ in the formula (7) is
preferably a hydrogen atom, a benzyl group, or a *—Si(R¹³)(R¹⁴)(R¹⁵) group (wherein * is a bonding position, R¹³ is a tert-butyl group, and R¹⁴ and R¹⁵ are each a methyl group or a phenyl group), or a *—CO—R²⁰ group (wherein * is a bonding position, and R²⁰ is an alkyl group having 1 to 9 carbon atoms),
more preferably a hydrogen atom, a *—Si(R¹³)(R¹⁴)(R¹⁵) group (wherein * is a bonding position, R¹³ is a tert-butyl group, and R¹⁴ and R¹⁵ are each a methyl group or a phenyl group), or a *—CO—R²⁰ group (wherein * is a bonding position, and R²⁰ is an alkyl group having 1 to 9 carbon atoms),
further preferably a hydrogen atom, a *—Si(R¹³)(R¹⁴)(R¹⁵) group (wherein * is a bonding position, R¹³ is a tert-butyl group, and R¹⁴ and R¹⁵ are each a methyl group), or a *—CO—R²⁰ group (wherein * is a bonding position, and R²⁰ is an alkyl group having 1 to 9 carbon atoms).

Group (7) is preferably a monovalent group derived from intermediate 50, deprotected intermediate 50 (i.e., compound in which the tert-butyldimethylsilyloxy group of intermediate 50 is replaced by a hydroxy group), intermediate 51, or intermediate 52 mentioned below.

R²¹ and R²² in the formula (8) are preferably each independently a hydrogen atom, a benzyl group, or a *—Si(R¹³)(R¹⁴)(R¹⁵) group (wherein * is a bonding position, R¹³ is a tert-butyl group, and R¹⁴ and R¹⁵ are each a methyl group or a phenyl group), more preferably each independently a hydrogen atom or a *—Si(R¹³)(R¹⁴)(R¹⁵) group (wherein * is a bonding position, R¹³ is a tert-butyl group, and R¹⁴ and R¹⁵ are each a methyl group or a phenyl group), further preferably each independently a hydrogen atom or a *—Si(R¹³)(R¹⁴)(R¹⁵) group (wherein * is a bonding position, R¹³ is a tert-butyl group, and R¹⁴ and R¹⁵ are each a methyl group).

Group (8) is preferably a monovalent group derived from intermediate 48 or deprotected intermediate 48 (i.e., a compound in which the tert-butyldimethylsilyloxy group of intermediate 48 is replaced by a hydroxy group) mentioned below.

R²³ in the formula (9) is
preferably a hydrogen atom, a benzyl group, or a *—Si(R¹³)(R¹⁴)(R¹⁵) group (wherein * is a bonding position, R¹³ is a tert-butyl group, and R¹⁴ and R¹⁵ are each a methyl group or a phenyl group),
more preferably a hydrogen atom or *—Si(R¹³)(R¹⁴)(R¹⁵) group (wherein * is a bonding position, R¹³ is a tert-butyl group, and R¹⁴ and R¹⁵ are each a methyl group or a phenyl group),
further preferably a hydrogen atom or a *—Si(R¹³)(R¹⁴)(R¹⁵) group (wherein * is a bonding position, R¹³ is a tert-butyl group, and R¹⁴ and R¹⁵ are each a methyl group).

Group (9) is preferably a monovalent group derived from intermediate 45 or deprotected intermediate 45 (i.e., a compound in which the tert-butyldimethylsilyloxy group of intermediate 45 is replaced by a hydroxy group) mentioned below.

R²⁴ in the formula (10) is preferably an alkylene group having 1 to 10 carbon atoms.

R²⁵ in the formula (10) is preferably an alkyl group having 1 to 30 carbon atoms.

Group (10) is preferably a monovalent group derived from intermediate 49 mentioned below.

R²⁶ in the formula (11) is preferably an alkylene group having 1 to 10 carbon atoms.

R²⁷ and R²⁸ in the formula (11) are preferably each independently an alkyl group having 2 to 10 carbon atoms or an alkenyl group having 2 to 10 carbon atoms, more preferably each independently an alkenyl group having 2 to 10 carbon atoms.

Group (11) is preferably a monovalent group derived from intermediate 54 mentioned below.

R²⁹ in the formula (12) is, as mentioned above, an alkylene group having 1 to 10 carbon atoms.

R³⁰ and R³¹ in the formula (12) are each independently an alkyl group having 1 to 10 carbon atoms.

Group (12) is preferably a monovalent group derived from intermediate 30 or intermediate 31 mentioned below.

Group (xiiib), which is one of the options for R^{3b} in formula (1), is preferably an alkyl group having 1 to 30 carbon atoms or an alkenyl group having 2 to 20 carbon atoms, in which one ethylene group in the aforementioned alkyl group is optionally replaced by one ester bond.

Group (xiiib) is more preferably a monovalent group derived from intermediate 53 mentioned below, or oleic acid, further preferably a monovalent group derived from oleic acid. In the present specification, the “monovalent group derived from oleic acid” means a monovalent group having a structure obtained by removing a carboxy group from oleic acid (i.e., (Z)-8-heptadecenyl group).

Group (xivb), which is one of the options for R^{3b} in formula (1), is as mentioned above, R^{3c}—CO—(CH₂)ₚ— group (wherein R^{3c} is a residue of a liposoluble vitamin having a hydroxy group or a residue of a sterol derivative having a hydroxy group, and p is an integer of 1 to 8). The p is preferably 2 or 3. The liposoluble vitamin having a hydroxy group is preferably a tocopherol. The sterol derivative having a hydroxy group is preferably a cholesterol or a cholestanol, more preferably cholesterol.

Table 1-1 to Table 1-9 show the names and structures of the intermediates.

TABLE 1-1

| name | structure |
|---|---|
| intermediate 1 | 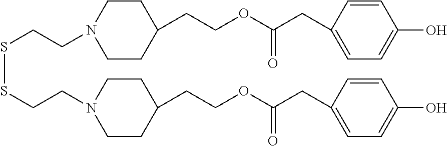 [SMILES: O=C(Cc1ccc(O)cc1)OCCC1CCN(CCSSCCN2CCC(CCOC(=O)Cc3ccc(O)cc3)CC2)CC1] |
| intermediate 2 | 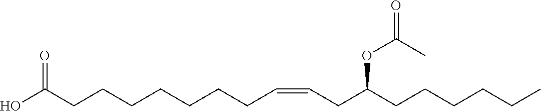 [SMILES: CCCCCC[C@@H](C/C=C\CCCCCCCC(=O)O)OC(C)=O] |
| intermediate 3 | 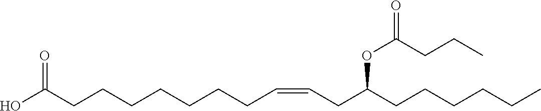 [SMILES: CCCCCC[C@@H](C/C=C\CCCCCCCC(=O)O)OC(=O)CCC] |
| intermediate 4 | 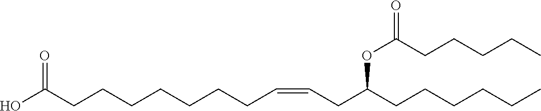 [SMILES: CCCCCC[C@@H](C/C=C\CCCCCCCC(=O)O)OC(=O)CCCCC] |
| intermediate 5 | 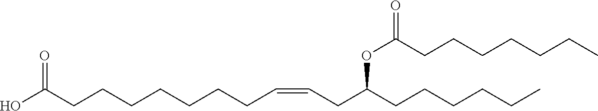 [SMILES: CCCCCCCC(=O)O[C@H](C/C=C\CCCCCCCC(=O)O)CCCCCC] |
| intermediate 6 | 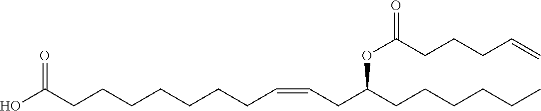 [SMILES: C=CCCCC(=O)O[C@H](C/C=C\CCCCCCCC(=O)O)CCCCCC] |
| intermediate 7 | 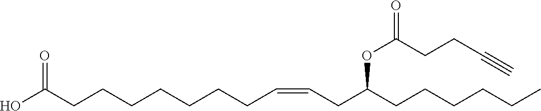 [SMILES: C#CCCC(=O)O[C@H](C/C=C\CCCCCCCC(=O)O)CCCCCC] |
| intermediate 8 | 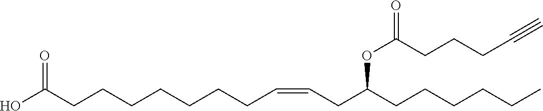 [SMILES: C#CCCCC(=O)O[C@H](C/C=C\CCCCCCCC(=O)O)CCCCCC] |
| intermediate 9 | 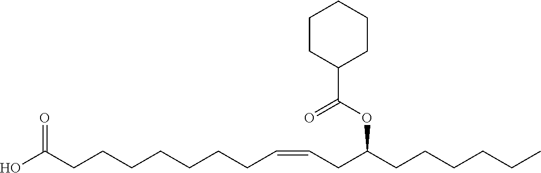 [SMILES: CCCCCC[C@@H](C/C=C\CCCCCCCC(=O)O)OC(=O)C1CCCCC1] |
| intermediate 10 | 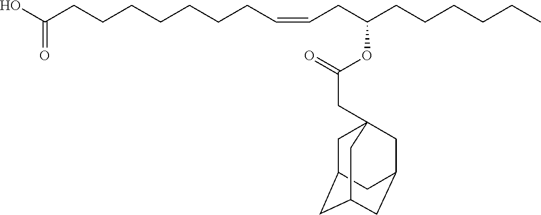 [SMILES: CCCCCC[C@@H](C/C=C\CCCCCCCC(=O)O)OC(=O)CC12CC3CC(CC(C3)C1)C2] |

TABLE 1-2

| name | structure |
|---|---|
| intermediate 11 | 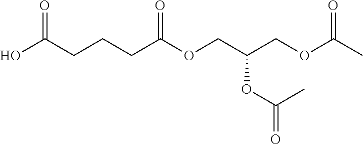 [SMILES: CC(=O)OC[C@@H](COC(=O)CCCC(=O)O)OC(C)=O] |
| intermediate 12 | 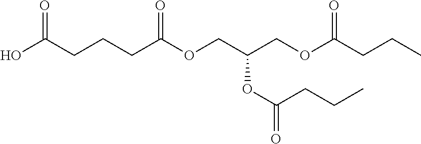 [SMILES: CCCC(=O)OC[C@@H](COC(=O)CCCC(=O)O)OC(=O)CCC] |
| intermediate 13 | 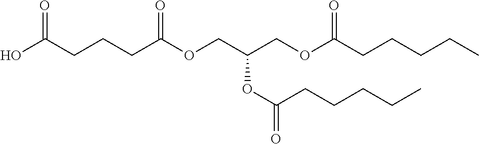 [SMILES: CCCCCC(=O)OC[C@@H](COC(=O)CCCC(=O)O)OC(=O)CCCCC] |
| intermediate 14 | 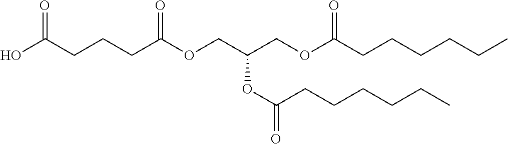 [SMILES: CCCCCCC(=O)OC[C@@H](COC(=O)CCCC(=O)O)OC(=O)CCCCCC] |
| intermediate 15 | 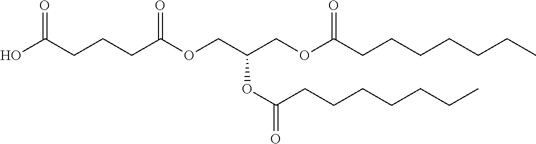 [SMILES: CCCCCCCC(=O)OC[C@@H](COC(=O)CCCC(=O)O)OC(=O)CCCCCCC] |
| intermediate 16 | 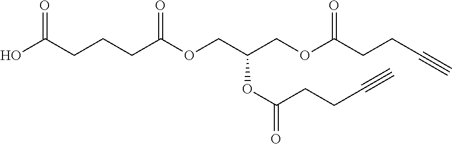 [SMILES: C#CCCC(=O)OC[C@@H](COC(=O)CCCC(=O)O)OC(=O)CCC#C] |
| intermediate 17 | 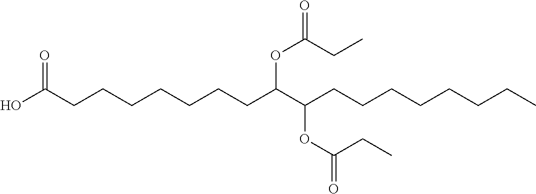 [SMILES: CCCCCCCCC(OC(=O)CC)C(CCCCCCCC(=O)O)OC(=O)CC] |

TABLE 1-3

| name | structure |
|---|---|
| intermediate 18 | 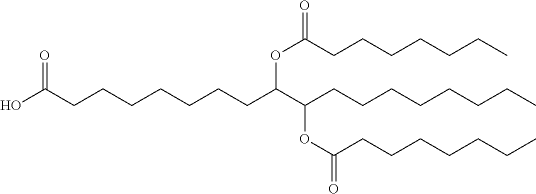 [SMILES: CCCCCCCCC(OC(=O)CCCCCCC)C(CCCCCCCC(=O)O)OC(=O)CCCCCCC] |
| intermediate 19 | 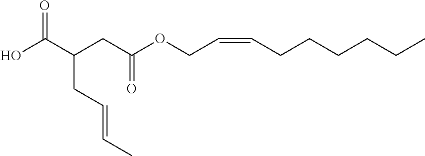 [SMILES: C/C=C/CC(CC(=O)OC/C=C\CCCCCC)C(=O)O] |
| intermediate 20 | 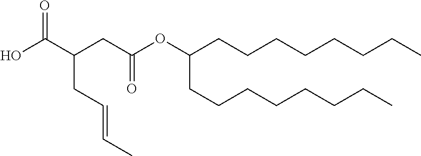 [SMILES: C/C=C/CC(CC(=O)OC(CCCCCCCC)CCCCCCCC)C(=O)O] |
| intermediate 21 | 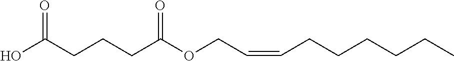 [SMILES: CCCCCC/C=C\COC(=O)CCCC(=O)O] |
| intermediate 22 | 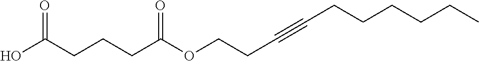 [SMILES: CCCCCCC#CCCOC(=O)CCCC(=O)O] |
| intermediate 23 | 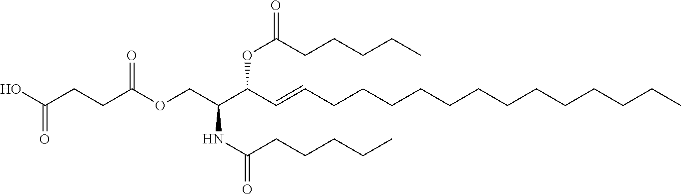 [SMILES: CCCCCCCCCCCCC/C=C/[C@@H](OC(=O)CCCCC)[C@H](COC(=O)CCC(=O)O)NC(=O)CCCCC] |
| intermediate 24 | 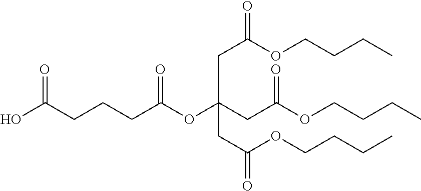 [SMILES: CCCCOC(=O)CC(CC(=O)OCCCC)(CC(=O)OCCCC)OC(=O)CCCC(=O)O] |
| intermediate 25 | 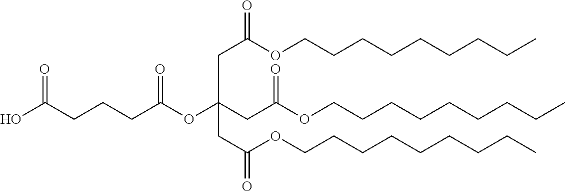 [SMILES: CCCCCCCCCOC(=O)CC(CC(=O)OCCCCCCCCC)(CC(=O)OCCCCCCCCC)OC(=O)CCCC(=O)O] |

TABLE 1-4

| name | structure |
|---|---|
| intermediate 26 | 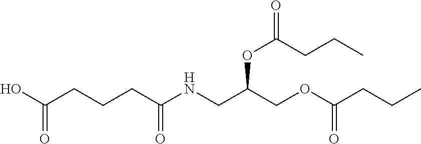 [SMILES: CCCC(=O)OC[C@@H](CNC(=O)CCCC(=O)O)OC(=O)CCC] |
| intermediate 27 | 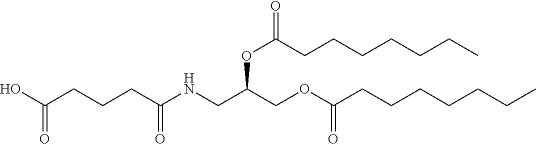 [SMILES: CCCCCCCC(=O)OC[C@@H](CNC(=O)CCCC(=O)O)OC(=O)CCCCCCC] |
| intermediate 28 | 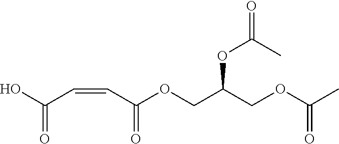 [SMILES: CC(=O)OC[C@@H](COC(=O)/C=C\C(=O)O)OC(C)=O] |
| intermediate 29 | 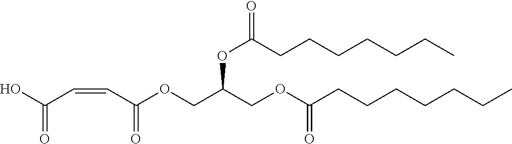 [SMILES: CCCCCCCC(=O)OC[C@@H](COC(=O)/C=C\C(=O)O)OC(=O)CCCCCCC] |
| intermediate 30 | 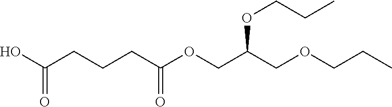 [SMILES: CCCOC[C@@H](COC(=O)CCCC(=O)O)OCCC] |
| intermediate 31 | 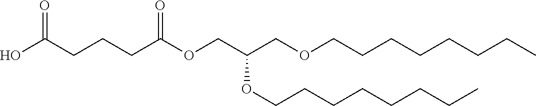 [SMILES: CCCCCCCCOC[C@@H](COC(=O)CCCC(=O)O)OCCCCCCCC] |
| intermediate 32 | 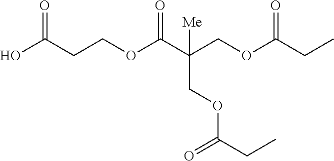 [SMILES: CCC(=O)OCC(C)(COC(=O)CC)C(=O)OCCC(=O)O] |
| intermediate 33 | 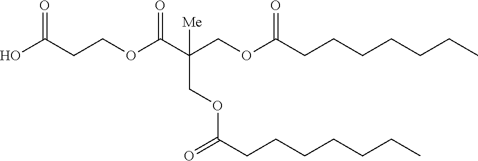 [SMILES: CCCCCCCC(=O)OCC(C)(COC(=O)CCCCCCC)C(=O)OCCC(=O)O] |

TABLE 1-5

| name | structure |
|---|---|
| intermediate 34 | 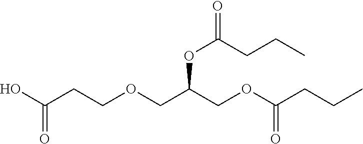 [SMILES: CCCC(=O)OC[C@@H](COCCC(=O)O)OC(=O)CCC] |
| intermediate 35 | 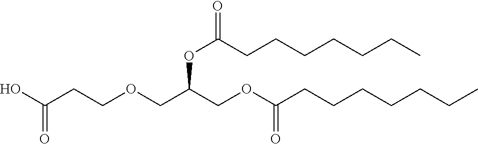 [SMILES: CCCCCCCC(=O)OC[C@@H](COCCC(=O)O)OC(=O)CCCCCCC] |
| intermediate 36 | 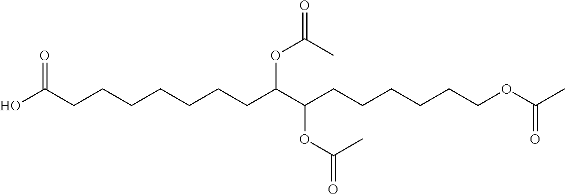 [SMILES: CC(=O)OCCCCCCC(OC(C)=O)C(CCCCCCCC(=O)O)OC(C)=O] |
| intermediate 37 | 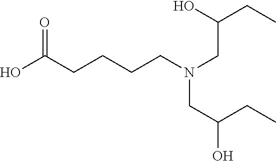 [SMILES: CCC(O)CN(CCCCC(=O)O)CC(O)CC] |
| intermediate 38 | 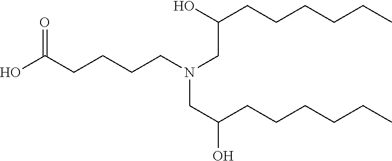 [SMILES: CCCCCCC(O)CN(CCCCC(=O)O)CC(O)CCCCCC] |
| intermediate 39 | 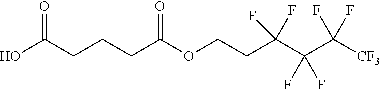 [SMILES: O=C(O)CCCC(=O)OCCC(F)(F)C(F)(F)C(F)(F)C(F)(F)F] |
| intermediate 40 | 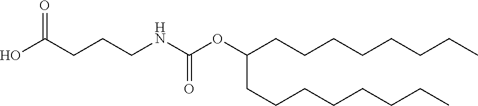 [SMILES: CCCCCCCCC(CCCCCCCC)OC(=O)NCCCC(=O)O] |
| intermediate 41 | 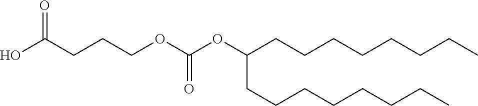 [SMILES: CCCCCCCCC(CCCCCCCC)OC(=O)OCCCC(=O)O] |

TABLE 1-6

| name | structure |
|---|---|
| intermediate 42 | 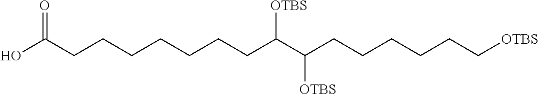 [SMILES: CC(C)(C)[Si](C)(C)OCCCCCCC(O[Si](C)(C)C(C)(C)C)C(CCCCCCCC(=O)O)O[Si](C)(C)C(C)(C)C] |
| intermediate 43 | 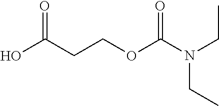 |
| intermediate 44 | 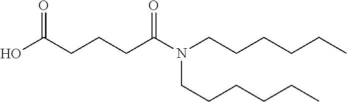 [SMILES: CCCCCCN(CCCCCC)C(=O)CCCC(=O)O] |
| intermediate 45 | 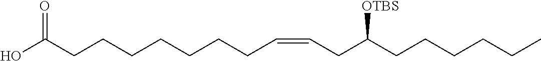 [SMILES: CCCCCC[C@@H](C/C=C\CCCCCCCC(=O)O)O[Si](C)(C)C(C)(C)C] |
| intermediate 47 | 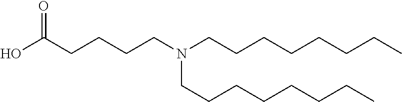 [SMILES: CCCCCCCCN(CCCCCCCC)CCCCC(=O)O] |
| intermediate 48 | 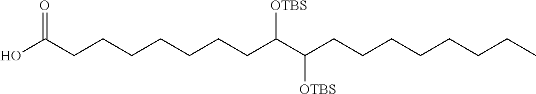 [SMILES: CCCCCCCCC(O[Si](C)(C)C(C)(C)C)C(CCCCCCCC(=O)O)O[Si](C)(C)C(C)(C)C] |
| intermediate 49 | 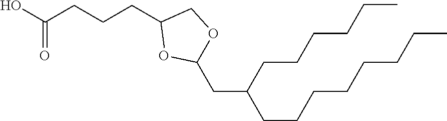 [SMILES: CCCCCCCCC(CCCCCC)CC1OCC(CCCC(=O)O)O1] |
| intermediate 50 | 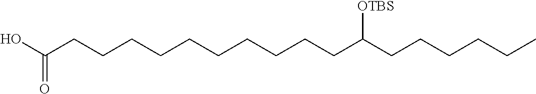 [SMILES: CCCCCCC(CCCCCCCCCCC(=O)O)O[Si](C)(C)C(C)(C)C] |
| intermediate 51 | 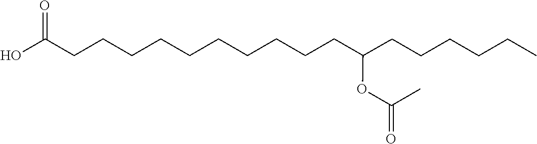 [SMILES: CCCCCCC(CCCCCCCCCCC(=O)O)OC(C)=O] |

TABLE 1-7

| name | structure |
|---|---|
| intermediate 52 | 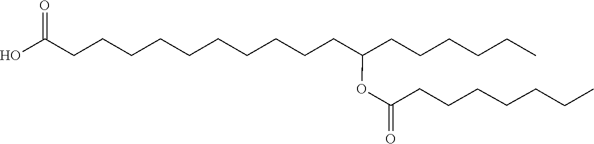 [SMILES: CCCCCCCC(=O)OC(CCCCCC)CCCCCCCCCCC(=O)O] |
| intermediate 53 | 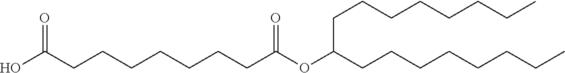 [SMILES: CCCCCCCCC(CCCCCCCC)OC(=O)CCCCCCCC(=O)O] |
| intermediate 54 | 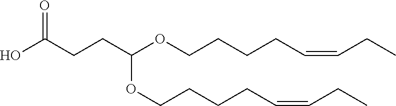 [SMILES: CC/C=C\CCCCOC(CCC(=O)O)OCCCC/C=C\CC] |
| intermediate 55 | 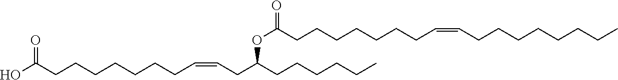 [SMILES: CCCCCCCC/C=C\CCCCCCCC(=O)O[C@H](C/C=C\CCCCCCCC(=O)O)CCCCCC] |
| intermediate 57 | 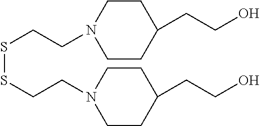 [SMILES: OCCC1CCN(CCSSCCN2CCC(CCO)CC2)CC1] |
| intermediate 58 | 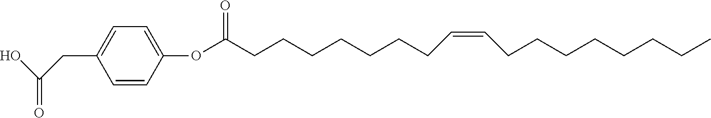 [SMILES: CCCCCCCC/C=C\CCCCCCCC(=O)Oc1ccc(CC(=O)O)cc1] |
| intermediate 59 | 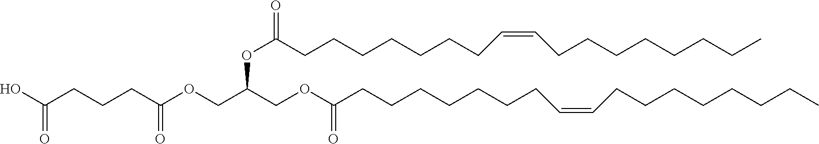 [SMILES: CCCCCCCC/C=C\CCCCCCCC(=O)OC[C@@H](COC(=O)CCCC(=O)O)OC(=O)CCCCCCC/C=C\CCCCCCCC] |
| intermediate 60 | 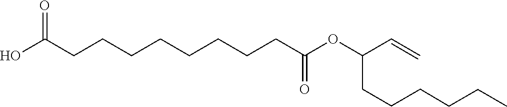 [SMILES: C=CC(CCCCCC)OC(=O)CCCCCCCCC(=O)O] |
| intermediate 61 | 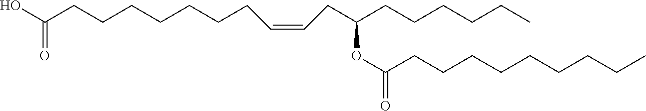 [SMILES: CCCCCCCCCC(=O)O[C@@H](C/C=C\CCCCCCCC(=O)O)CCCCCC] |
| intermediate 62 | 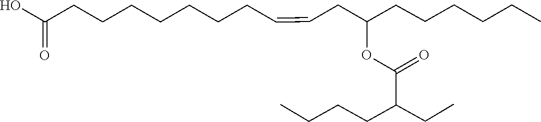 [SMILES: CCCCCCC(C/C=C\CCCCCCCC(=O)O)OC(=O)C(CC)CCCC] |

TABLE 1-8

| name | structure |
|---|---|
| intermediate 63 | 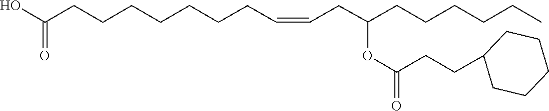 [SMILES: CCCCCCC(C/C=C\CCCCCCCC(=O)O)OC(=O)CCC1CCCCC1] |
| intermediate 64 | 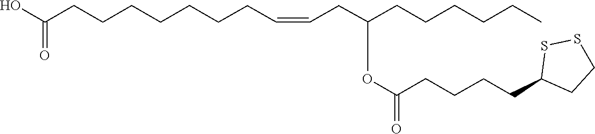 [SMILES: CCCCCCC(C/C=C\CCCCCCCC(=O)O)OC(=O)CCCC[C@@H]1CCSS1] |
| intermediate 65 | 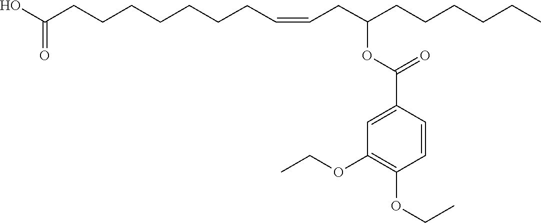 [SMILES: CCCCCCC(C/C=C\CCCCCCCC(=O)O)OC(=O)c1ccc(OCC)c(OCC)c1] |
| intermediate 66 | 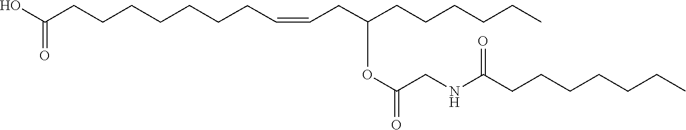 [SMILES: CCCCCCCC(=O)NCC(=O)OC(C/C=C\CCCCCCCC(=O)O)CCCCCC] |
| intermediate 67 | 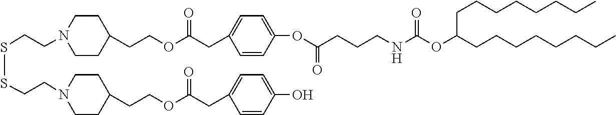 [SMILES: CCCCCCCCC(CCCCCCCC)OC(=O)NCCCC(=O)Oc1ccc(CC(=O)OCCC2CCN(CCSSCCN3CCC(CCOC(=O)Cc4ccc(O)cc4)CC3)CC2)cc1] |
| intermediate 68 | 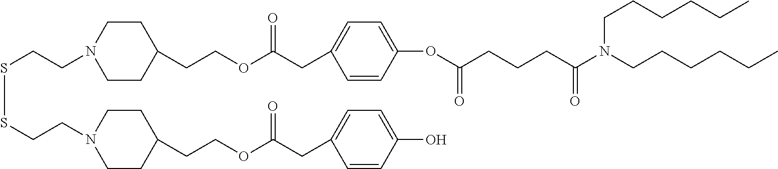 [SMILES: CCCCCCN(CCCCCC)C(=O)CCCC(=O)Oc1ccc(CC(=O)OCCC2CCN(CCSSCCN3CCC(CCOC(=O)Cc4ccc(O)cc4)CC3)CC2)cc1] |
| intermediate 69 | 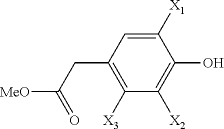 |
| intermediate 70 | 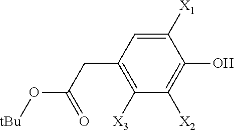 |

TABLE 1-9

| name | structure |
|---|---|
| intermediate 71 | 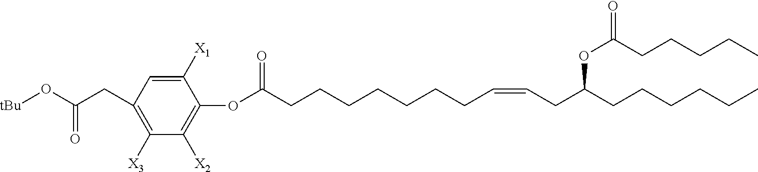 |
| intermediate 72 | 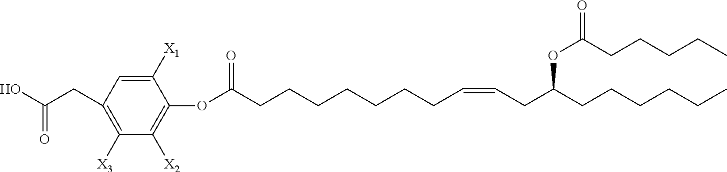 |
| intermediate 73 | 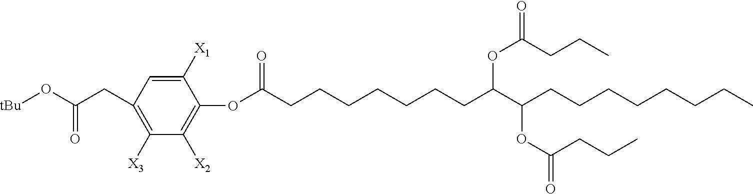 |
| intermediate 74 | 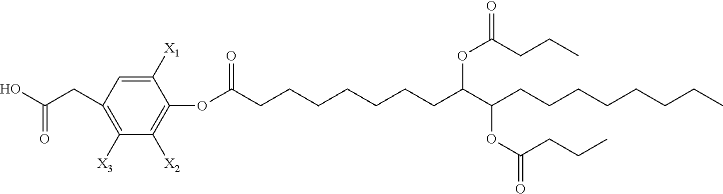 |
| intermediate 75 | 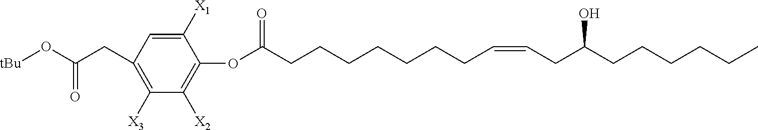 |
| intermediate 76 | 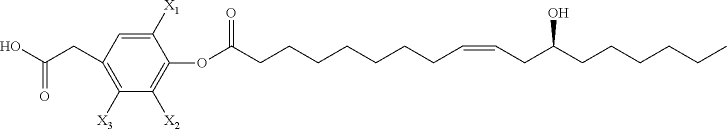 |

Examples of the cationic lipid represented by the formula (1) include the lipids shown in Table 2-1 to Table 2-6.

TABLE 2-1

| No. | structure |
|---|---|
| 1 | 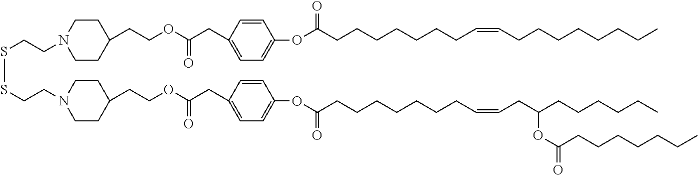 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCCCCCC)cc4)CC3)CC2)cc1] |
| 2 | 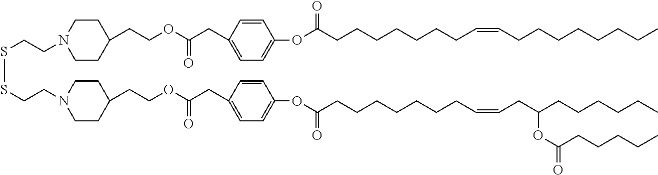 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCCCC)cc4)CC3)CC2)cc1] |
| 3 | 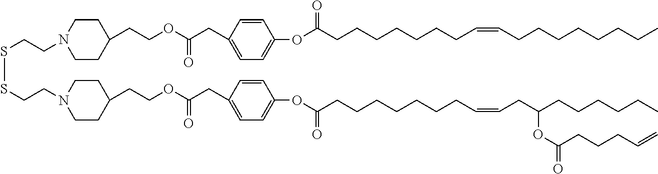 [SMILES: C=CCCCC(=O)OC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1)CCCCCC] |
| 4 | 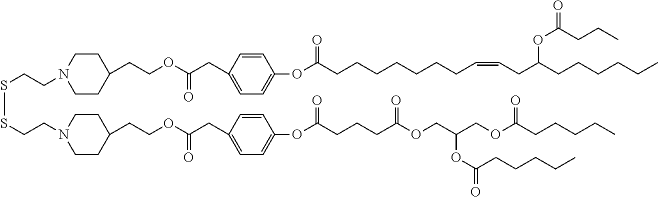 [SMILES: CCCCCCC(C/C=C\CCCCCCCC(=O)Oc1ccc(CC(=O)OCCC2CCN(CCSSCCN3CCC(CCOC(=O)Cc4ccc(OC(=O)CCCC(=O)OCC(COC(=O)CCCCC)OC(=O)CCCCC)cc4)CC3)CC2)cc1)OC(=O)CCC] |
| 5 | 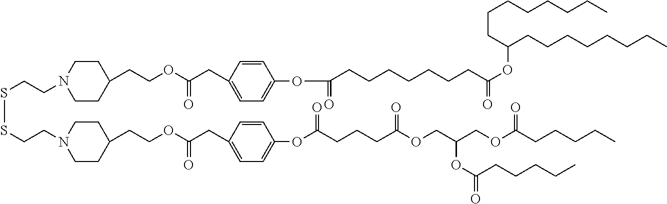 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCC(=O)OCC(COC(=O)CCCCC)OC(=O)CCCCC)cc4)CC3)CC2)cc1] |

TABLE 2-2

| No. | structure |
|---|---|
| 6 | 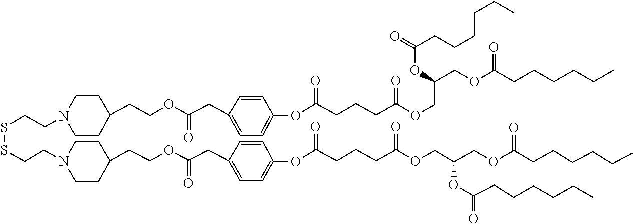 [SMILES: CCCCCCC(=O)OC[C@@H](COC(=O)CCCC(=O)Oc1ccc(CC(=O)OCCC2CCN(CCSSCCN3CCC(CCOC(=O)Cc4ccc(OC(=O)CCCC(=O)OC[C@H](COC(=O)CCCCCC)OC(=O)CCCCCC)cc4)CC3)CC2)cc1)OC(=O)CCCCCC] |
| 7 | 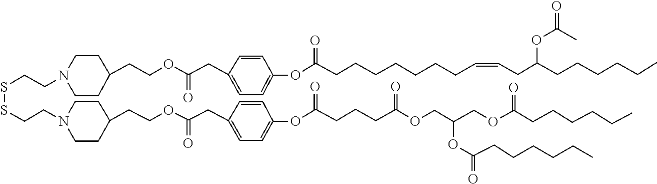 [SMILES: CCCCCCC(=O)OCC(COC(=O)CCCC(=O)Oc1ccc(CC(=O)OCCC2CCN(CCSSCCN3CCC(CCOC(=O)Cc4ccc(OC(=O)CCCCCCC/C=C\CC(CCCCCC)OC(C)=O)cc4)CC3)CC2)cc1)OC(=O)CCCCCC] |
| 8 | 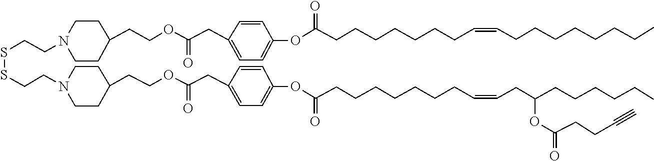 [SMILES: C#CCCC(=O)OC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1)CCCCCC] |
| 9 | 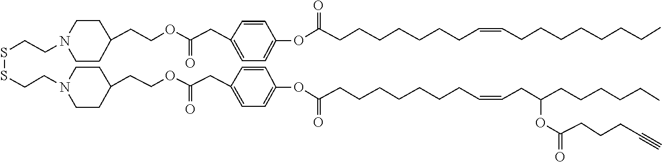 [SMILES: C#CCCCC(=O)OC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1)CCCCCC] |
| 10 | 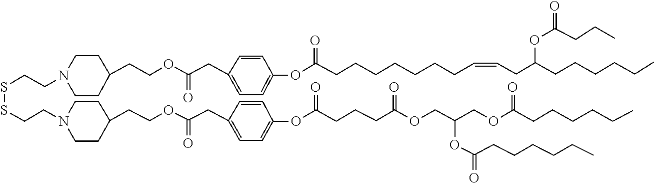 [SMILES: CCCCCCC(=O)OCC(COC(=O)CCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCC)cc4)CC3)CC2)cc1)OC(=O)CCCCCC] |

TABLE 2-3

| No. | structure |
|---|---|
| 11 | 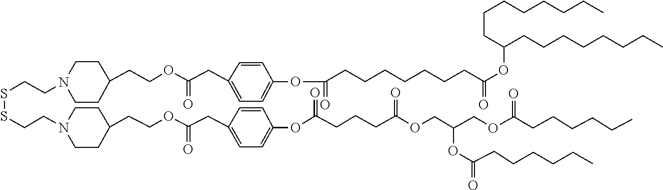 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCC(=O)OCC(COC(=O)CCCCCC)OC(=O)CCCCCC)cc4)CC3)CC2)cc1] |
| 12 | 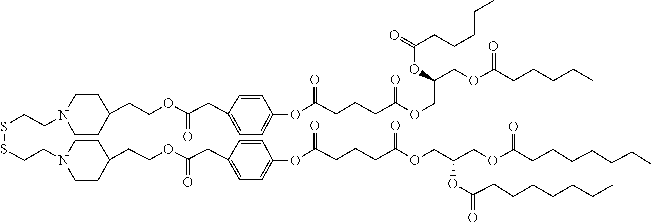 [SMILES: CCCCCCCC(=O)OC[C@@H](COC(=O)CCCC(=O)Oc1ccc(CC(=O)OCCC2CCN(CCSSCCN3CCC(CCOC(=O)Cc4ccc(OC(=O)CCCC(=O)OC[C@H](COC(=O)CCCCC)OC(=O)CCCCC)cc4)CC3)CC2)cc1)OC(=O)CCCCCCC] |
| 13 | 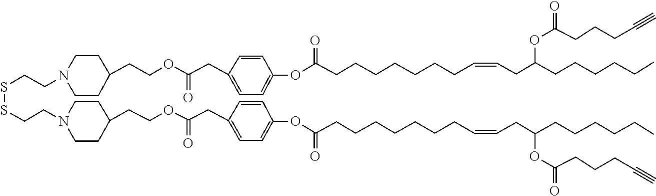 [SMILES: C#CCCCC(=O)OC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCCC#C)cc4)CC3)CC2)cc1)CCCCCC] |
| 14 | 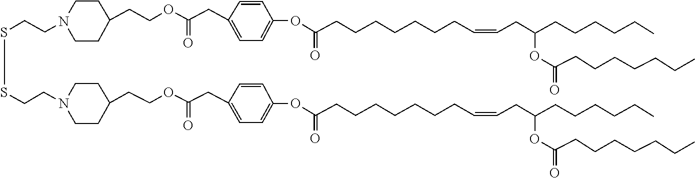 [SMILES: CCCCCCCC(=O)OC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCCCCCC)cc4)CC3)CC2)cc1)CCCCCC] |
| 15 | 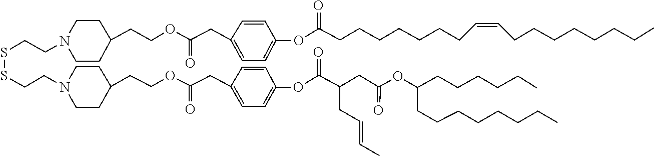 [SMILES: C/C=C/CC(CC(=O)OC(CCCCCC)CCCCCCCC)C(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1] |

TABLE 2-4

| No. | structure |
|---|---|
| 16 | 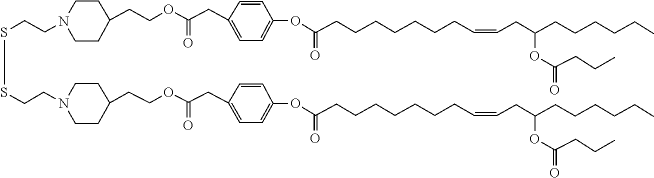 [SMILES: CCCCCCC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCC)cc4)CC3)CC2)cc1)OC(=O)CCC] |
| 17 | 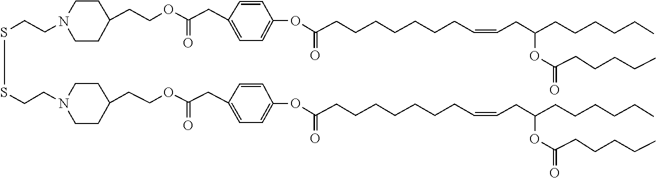 [SMILES: CCCCCCC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCCCC)cc4)CC3)CC2)cc1)OC(=O)CCCCC] |
| 18 | 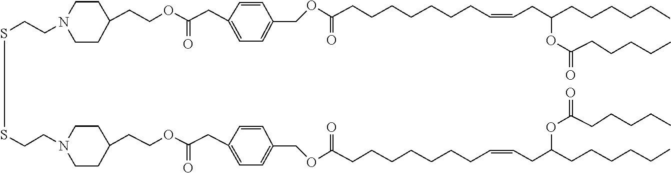 [SMILES: CCCCCCC(C/C=C\CCCCCCCC(=O)OCc1ccc(CC(=O)OCCC2CCN(CCSSCCN3CCC(CCOC(=O)Cc4ccc(COC(=O)CCCCCCC/C=C\CC(CCCCCC)OC(=O)CCCCC)cc4)CC3)CC2)cc1)OC(=O)CCCCC] |
| 19 | 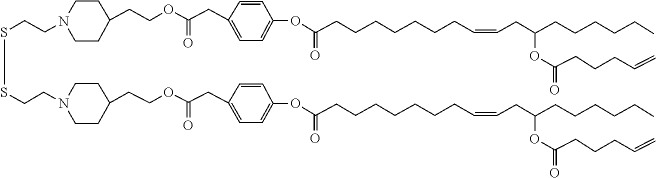 [SMILES: C=CCCCC(=O)OC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCCC=C)cc4)CC3)CC2)cc1)CCCCCC] |
| 20 | 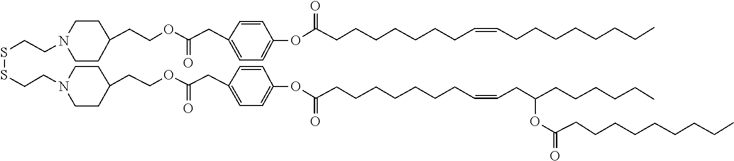 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCCCCCCCC)cc4)CC3)CC2)cc1] |

TABLE 2-5

| No. | structure |
|---|---|
| 21 | 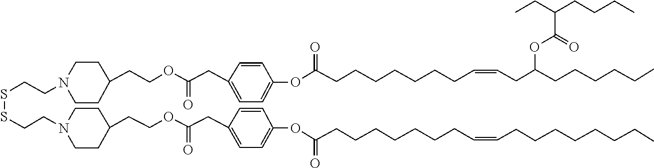 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)C(CC)CCCC)cc4)CC3)CC2)cc1] |
| 22 | 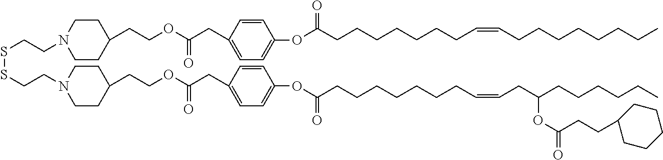 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCC5CCCCC5)cc4)CC3)CC2)cc1] |
| 23 | 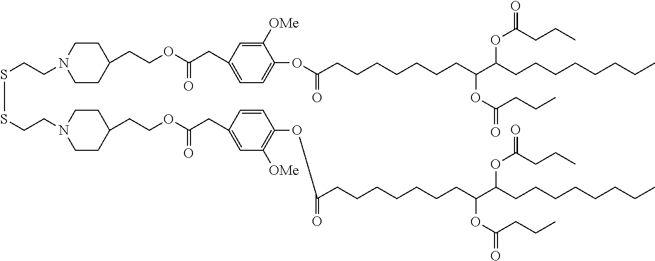 [SMILES: CCCCCCCCC(OC(=O)CCC)C(CCCCCCCC(=O)Oc1ccc(CC(=O)OCCC2CCN(CCSSCCN3CCC(CCOC(=O)Cc4ccc(OC(=O)CCCCCCCC(OC(=O)CCC)C(CCCCCCCC)OC(=O)CCC)c(OC)c4)CC3)CC2)cc1OC)OC(=O)CCC] |
| 24 | 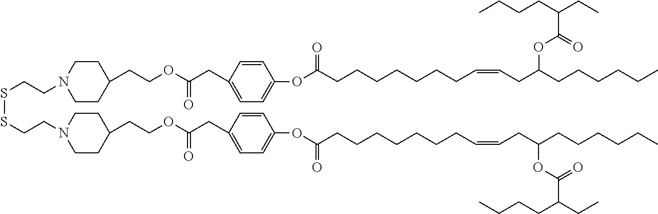 [SMILES: CCCCCCC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)C(CC)CCCC)cc4)CC3)CC2)cc1)OC(=O)C(CC)CCCC] |
| 25 | 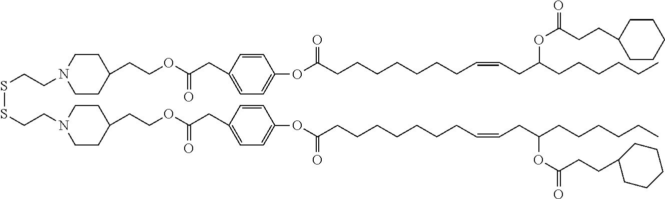 [SMILES: CCCCCCC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCC5CCCCC5)cc4)CC3)CC2)cc1)OC(=O)CCC1CCCCC1] |

TABLE 2-6

| No. | structure |
|---|---|
| 26 | 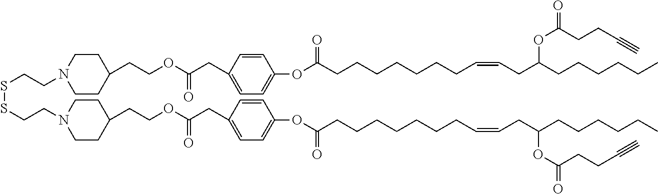 [SMILES: C#CCCC(=O)OC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCC#C)cc4)CC3)CC2)cc1)CCCCCC] |
| 27 | 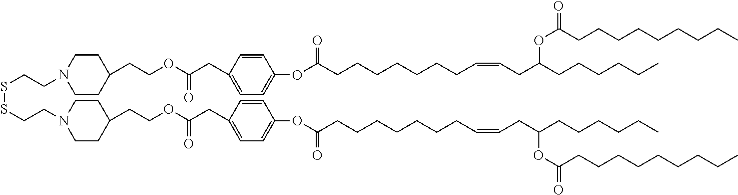 [SMILES: CCCCCCCCCC(=O)OC(C/C=C\CCCCCCCC(=O)Oc1ccc(CC(=O)OCCC2CCN(CCSSCCN3CCC(CCOC(=O)Cc4ccc(OC(=O)CCCCCCC/C=C\CC(CCCCCC)OC(=O)CCCCCCCCC)cc4)CC3)CC2)cc1)CCCCCC] |
| 28 | 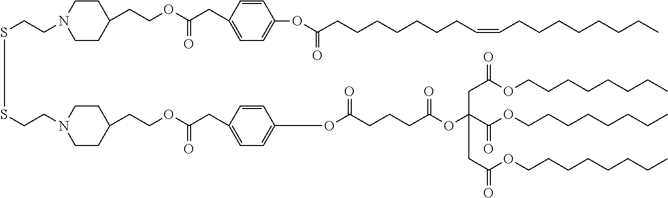 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCC(=O)OC(CC(=O)OCCCCCCCC)(CC(=O)OCCCCCCCC)C(=O)OCCCCCCCC)cc4)CC3)CC2)cc1] |
| 29 | 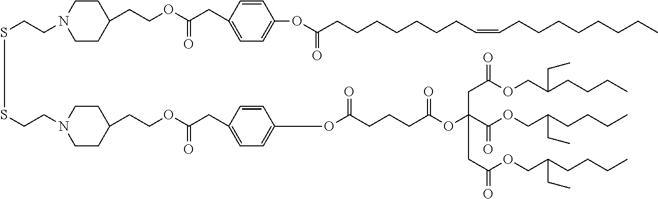 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCC(=O)OC(CC(=O)OCC(CC)CCCC)(CC(=O)OCC(CC)CCCC)C(=O)OCC(CC)CCCC)cc4)CC3)CC2)cc1] |

Specific examples of the ionic lipid include the following O-Ph-P3C1, O-Ph-P4C1, O-Ph-P4C2 (or SS-OP), O-Bn-P4C2, E-Ph-P4C2 (or SS-EP), L-Ph-P4C2, HD-Ph-P4C2, O-Ph-amide-P4C2, O-Ph-C3M, B-2, B-2-5, TS-P4C2 (or SS-EC), L-P4C2, and O-P4C2.

TABLE 2-7

| name of | |
|---|---|
| cationic lipid | structure |
| O—Ph—P3C1 | 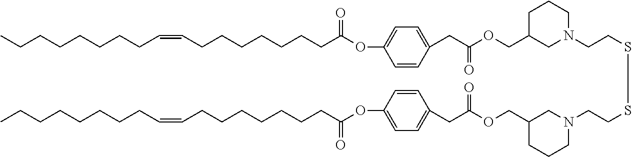 [SMILES: CCCCCCCC/C=C\CCCCCCCC(=O)Oc1ccc(CC(=O)OCC2CCCN(CCSSCCN3CCCC(COC(=O)Cc4ccc(OC(=O)CCCCCCC/C=C\CCCCCCCC)cc4)C3)C2)cc1] |
| O—Ph—P4C1 | 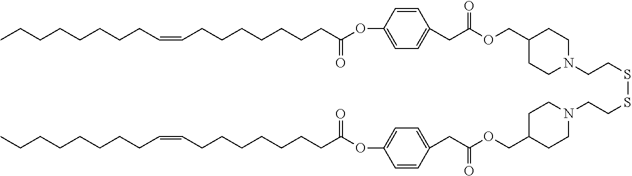 [SMILES: CCCCCCCC/C=C\CCCCCCCC(=O)Oc1ccc(CC(=O)OCC2CCN(CCSSCCN3CCC(COC(=O)Cc4ccc(OC(=O)CCCCCCC/C=C\CCCCCCCC)cc4)CC3)CC2)cc1] |
| O—Ph—P4C2 (or SS—OP) | 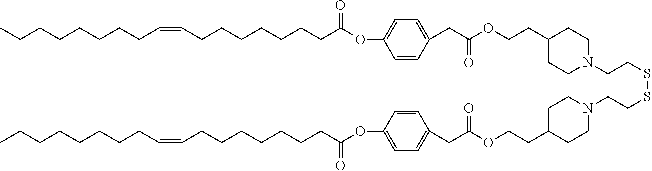 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1] |
| O—Bn—P4C2 | 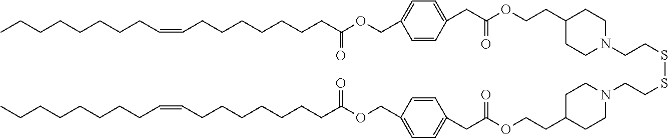 [SMILES: CCCCCCCC/C=C\CCCCCCCC(=O)OCc1ccc(CC(=O)OCCC2CCN(CCSSCCN3CCC(CCOC(=O)Cc4ccc(COC(=O)CCCCCCC/C=C\CCCCCCCC)cc4)CC3)CC2)cc1] |
| E—Ph—P4C2 (or SS—EP) | 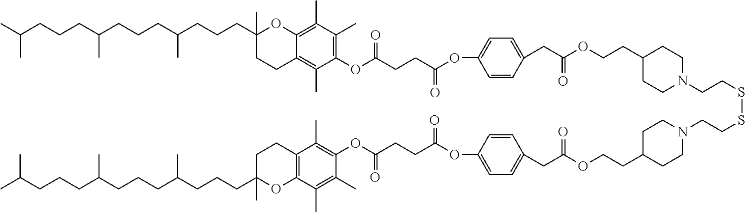 [SMILES: Cc1c(C)c2c(c(C)c1OC(=O)CCC(=O)Oc1ccc(CC(=O)OCCC3CCN(CCSSCCN4CCC(CCOC(=O)Cc5ccc(OC(=O)CCC(=O)Oc6c(C)c(C)c7c(c6C)CCC(C)(CCCC(C)CCCC(C)CCCC(C)C)O7)cc5)CC4)CC3)cc1)CCC(C)(CCCC(C)CCCC(C)CCCC(C)C)O2] |

TABLE 2-8

| name of | |
|---|---|
| cationic lipid | structure |
| L—Ph—P4C2 | 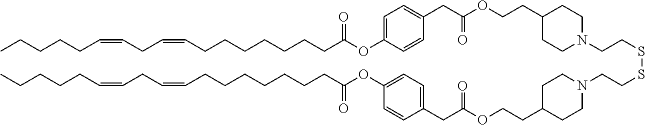 [SMILES: CCCCC/C=C\C/C=C\CCCCCCCC(=O)Oc1ccc(CC(=O)OCCC2CCN(CCSSCCN3CCC(CCOC(=O)Cc4ccc(OC(=O)CCCCCCC/C=C\C/C=C\CCCCC)cc4)CC3)CC2)cc1] |
| HD—Ph—P4C2 | 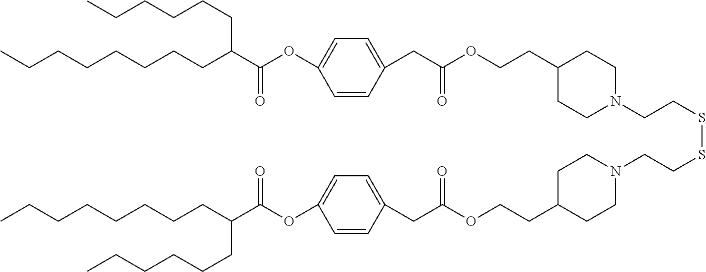 [SMILES: CCCCCCCCC(CCCCCC)C(=O)Oc1ccc(CC(=O)OCCC2CCN(CCSSCCN3CCC(CCOC(=O)Cc4ccc(OC(=O)C(CCCCCC)CCCCCCCC)cc4)CC3)CC2)cc1] |
| O—Ph-amide-P4C2 | 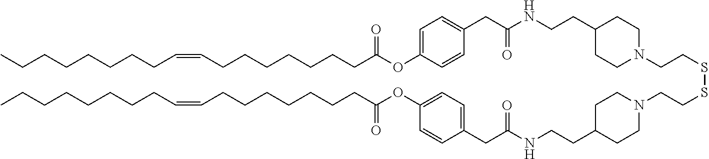 [SMILES: CCCCCCCC/C=C\CCCCCCCC(=O)Oc1ccc(CC(=O)NCCC2CCN(CCSSCCN3CCC(CCNC(=O)Cc4ccc(OC(=O)CCCCCCC/C=C\CCCCCCCC)cc4)CC3)CC2)cc1] |
| O—Ph—C3M | 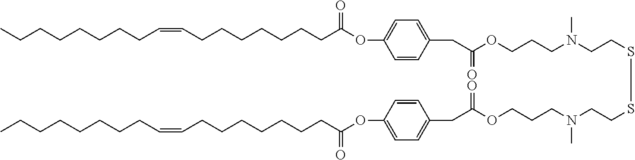 [SMILES: CCCCCCCC/C=C\CCCCCCCC(=O)Oc1ccc(CC(=O)OCCCN(C)CCSSCCN(C)CCCOC(=O)Cc2ccc(OC(=O)CCCCCCC/C=C\CCCCCCCC)cc2)cc1] |

TABLE 2-9

| name of | |
|---|---|
| cationic lipid | structure |
| B-2 | 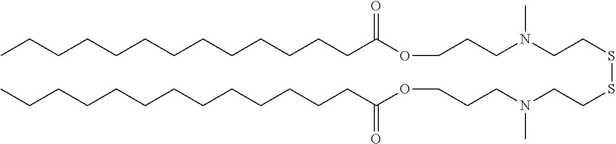 [SMILES: CCCCCCCCCCCCCC(=O)OCCCN(C)CCSSCCN(C)CCCOC(=O)CCCCCCCCCCCCC] |
| B-2-5 | 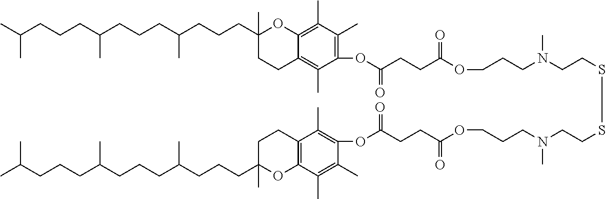 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2] |
| TS—P4C2 (or SS—EC) | 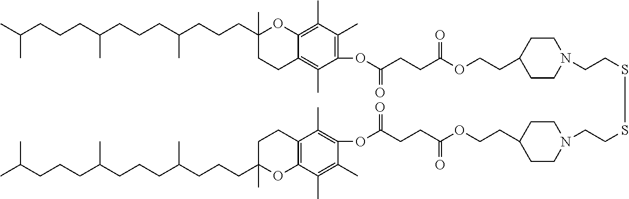 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] |
| L—P4C2 | 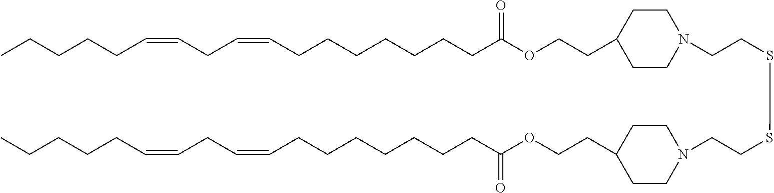 [SMILES: CCCCC/C=C\C/C=C\CCCCCCCC(=O)OCCC1CCN(CCSCSCCN2CCC(CCOC(=O)CCCCCCC/C=C\C/C=C\CCCCC)CC2)CC1] |
| O—P4C2 | 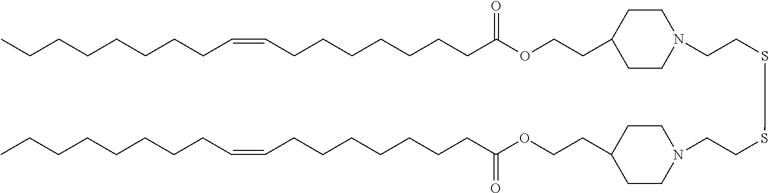 [SMILES: CCCCCCCC/C=C\CCCCCCCC(=O)OCCC1CCN(CCSCSCCN2CCC(CCOC(=O)CCCCCCC/C=C\CCCCCCCC)CC2)CC1] |

The production method of cationic lipid (1) is now explained.

Cationic lipid (1) has an —S—S— (disulfide) bond. Therefore, the production method of cationic lipid (1) includes (α) a method for obtaining cationic lipid (1) containing an —S—S— bond by producing R^{3a}—CO—O—Z^{a}—Y^{a}—R^{2a}—X^{a}—R^{1a}—SH (i.e., thiol compound), and R^{3b}—CO—O—Z^{b}—Y^{b}—R^{2b}—X^{b}—R^{1b}—SH (i.e., thiol compound) and then oxidizing (coupling) same, or (β) a method in which necessary parts are sequentially synthesized from a compound containing an —S—S— bond to finally obtain cationic lipid (1). It is preferably method (β). Specific examples of method (β) are described in WO 2024/203577 but the production method of cationic lipid (1) is not limited.

The formula (2A):

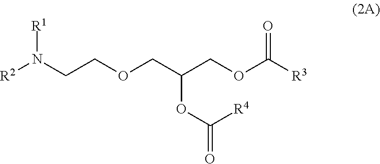

In the formula (2A), R¹ and R² are each independently an alkyl group having 1 to 6 carbon atoms, or any carbon atoms of R¹ and R² are optionally bonded to each other to form a heterocycle containing nitrogen atom, and
R³ and R⁴ are each independently an aliphatic hydrocarbon group having 1 to 35 carbon atoms optionally substituted by a substituent selected from Substituent Group α,
provided that at least one selected from the group consisting of R³ and R⁴ is an aliphatic hydrocarbon group having 5 to 35 carbon atoms optionally substituted by a substituent selected from Substituent Group α, wherein Substituent Group α consists of a hydroxy group, an alkoxy group, a sulfanyl group, an alkylthio group, and an alkoxycarbonyl group.

Examples of the alkyl group having 1 to 6 carbon atoms for R and R² include methyl group, ethyl group, n-propyl group, isopropyl group, n-butyl group, sec-butyl group, tert-butyl group, n-pentyl group, isopentyl group, 2-methylbutyl group, neopentyl group, 1-ethylpropyl group, n-hexyl group, 4-methylpentyl group, 3-methylpentyl group, 2-methylpentyl group, 1-methylpentyl group, 3,3-dimethylbutyl group, 2,2-dimethylbutyl group, 1,1-dimethylbutyl group, 1,2-dimethylbutyl group, 1,3-dimethylbutyl group, 2,3-dimethylbutyl group, and 2-ethylbutyl group.

Any carbon atoms of R¹ and R² are optionally bonded to each other, together with the nitrogen atom to which R¹ and R² are bonded, to form a heterocycle containing nitrogen atom. Examples of the heterocycle containing nitrogen atom include aziridine, azetidine, pyrrolidine, and piperidine.

R¹ and R² are each preferably an alkyl group having 2 to 6 carbon atoms, more preferably a linear alkyl group having 2 to 6 carbon atoms, further preferably ethyl group.

The aliphatic hydrocarbon group having 1 to 35 carbon atoms for R³ and R⁴ is a monovalent group having a structure obtained by removing one hydrogen atom from any carbon atom of a non-aromatic aliphatic compound consisting only of carbon and hydrogen having 1 to 35 carbon atoms.

Examples of the aliphatic hydrocarbon group having 1 to 35 carbon atoms include a linear or branched alkyl group having 5 to 35 carbon atoms such as n-pentyl, isopentyl, 3-methylpentyl, 1-methylpentyl, 3,3-dimethylbutyl, 2,2-dimethylbutyl, 1,1-dimethylbutyl, 1,3-dimethylbutyl, 2,3-dimethylbutyl, 4-methylheptyl, decyl, undecyl, dodecyl, tridecyl, tetradecyl, pentadecyl, hexadecyl, heptadecyl, octadecyl, nonadecyl, icosyl, henicosyl, docosyl, tricosyl, tatracosyl, pentacosyl, methyldecyl, 1-hexyloctyl, 1-hexylnonyl, 1-heptylnonyl, 1-hexyldecyl, 1-hexyldecyl, 1-methylundecyl, 1-(1,3,3-trimethylbutyl)-2,6,6-trimethylheptyl, 10-trimethylundecyl, 3,7,11-trimethyldodecyl, 6,10,14-trimethylpentadecan-2-yl, 2,6,10,14-tetramethylpentadecyl, 3,7,11,15-tetramethylhexadecyl, 11-(tert-butoxy)-11-oxoundecanyl, 13-(tert-butoxy)-13-oxotridecanyl, 17-(tert-butoxy)-17-oxoheptadecanyl, 2-(dodecylthio)ethanyl, and the like;
a linear or branched alkenyl group having 5 to 35 carbon atoms such as (Z)-non-8-enyl, (Z)-tridec-8-enyl, (Z)-tetradec-9-enyl, (Z)-pentadec-8-enyl, (Z)-hexadec-9-enyl, (Z)-heptadec-5-enyl, (Z)-octadec-6-enyl, (Z)-heptadec-8-enyl, (Z)-octadec-9-enyl, (E)-heptadec-8-enyl, (E)-octadec-9-enyl, (Z)-heptadec-10-enyl, (Z)-octadec-11-enyl, (8Z,11Z)-heptadeca-8,11-dienyl, (9Z,12Z)-octadeca-9,12-dienyl, (8Z,11Z,14Z)-octadeca-8,11,14-trienyl, (9Z,12Z,15Z)-octadeca-9,12,15-trienyl, (Z)-nonadec-10-enyl, (Z)-icos-11-enyl, (10Z,13Z)-nonadeca-10,13-dienyl, (11Z,14Z)-icosa-11,14-dienyl, 2,6,10-trimethylundeca-1,5,9-trienyl, 3,7,11-trimethyldodeca-2,6,10-trienyl, 2,6,10,14-tetramethylpentadec-1-enyl, 3,7,11,15-tetramethylhexadec-2-enyl, (Z)-henicos-12-enyl, and the like;
an alkynyl group having 5 to 35 carbon atoms such as dodec-11-ynyl, tridec-12-ynyl, pentadec-6-ynyl, hexadec-7-ynyl, pentadeca-4,6-diynyl, hexadeca-5,7-diynyl, heptadec-8-ynyl, octadec-9-ynyl, and the like; and
an alkyl group, alkenyl group, or alkynyl group having 1 to 4 carbon atoms such as methyl, ethyl, n-propyl, isopropyl, n-butyl, tert-butyl, and the like.

Either one of R³ or R⁴ or both of R³ and R⁴ are preferably a secondary alkyl group having 5 to 35 carbon atoms. In addition, either one of R³ or R⁴ or both of R³ and R⁴ are preferably an alkenyl group having 5 to 35 carbon atoms. It is more preferred that either one of R³ or R⁴ is a secondary alkyl group having 5 to 35 carbon atoms, and the other is an alkenyl group having 5 to 35 carbon atoms. When either one of R³ or R⁴ or both of R³ and R⁴ are a secondary alkyl group having 5 to 35 carbon atoms, or either one of R³ or R⁴ or both of R³ and R⁴ are an alkenyl group having 5 to 35 carbon atoms, the endosomal escape ability of lipid nanoparticles including the present embodiments can be further improved.

Examples of the secondary alkyl group having 5 to 35 carbon atoms include 1-hexyloctyl, 1-hexylnonyl, 1-heptylnonyl, 1-hexyldecyl, 1-methylundecyl, 1-(1,3,3-trimethylbutyl)-2,6,6-trimethylheptyl, and the like. Among these, 1-hexyloctyl, 1-hexylnonyl, 1-heptylnonyl, 1-hexyldecyl, 1-heptylnonyl, and 1-methylundecyl are preferred, and 1-hexylnonyl is more preferred.

The alkenyl group having 5 to 35 carbon atoms is preferably (Z)-heptadec-8-enyl or (8Z,11Z)-heptadeca-8,11-dienyl, and more preferably (Z)-heptadec-8-enyl.

It is preferred that both of R³ and R⁴ are (Z)-heptadec-8-enyl, or both of R³ and R⁴ are (8Z,11Z)-heptadeca-8,11-dienyl, or both of R³ and R⁴ are 1-hexylnonyl, or both of R³ and R⁴ are 1-(1,3,3-trimethylbutyl)-2,6,6-trimethylheptyl, or either one of R³ or R⁴ is 1-hexylnonyl, and the other is (Z)-heptadec-8-enyl. It is more preferred that both of R³ and R⁴ are 1-hexylnonyl, or both of R³ and R⁴ are 1-(1,3,3-trimethylbutyl)-2,6,6-trimethylheptyl, or either one of R³ or R⁴ is 1-hexylnonyl, and the other is (Z)-heptadec-8-enyl. It is further preferred that either one of R³ or R⁴ is 1-hexylnonyl, and the other is (Z)-heptadec-8-enyl.

The aliphatic hydrocarbon group having 1 to 35 carbon atoms for R³ and R⁴ is optionally substituted by a substituent selected from Substituent Group α. The term “optionally substituted” means that a hydrogen atom of the aliphatic hydrocarbon group may be substituted by a substituent selected from Substituent Group α. Substituent Group α consists of a hydroxy group, an alkoxy group, a sulfanyl group, an alkylthio group, and an alkoxycarbonyl group.

Examples of the cationic lipid represented by the formula (2A) include the lipids shown in Table 2-10 and Table 2-11.

TABLE 2-10

| No. | structure |
|---|---|
| 30 | 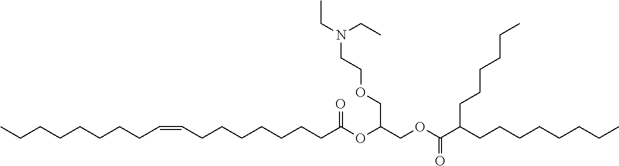 [SMILES: CCCCCCCC/C=C\CCCCCCCC(=O)OC(COCCN(CC)CC)COC(=O)C(CCCCCC)CCCCCCCC] |
| 31 | 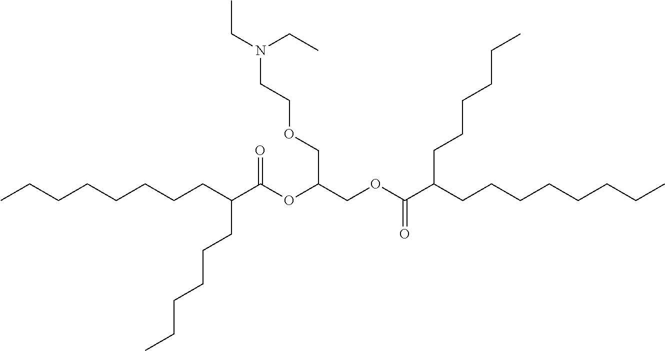 [SMILES: CCCCCCCCC(CCCCCC)C(=O)OCC(COCCN(CC)CC)OC(=O)C(CCCCCC)CCCCCCCC] |
| 32 | 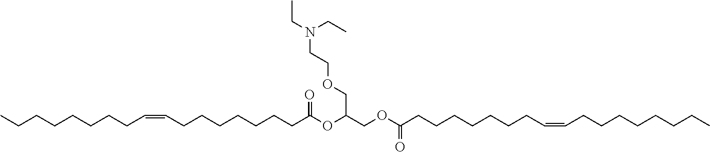 [SMILES: CCCCCCCC/C=C\CCCCCCCC(=O)OCC(COCCN(CC)CC)OC(=O)CCCCCCC/C=C\CCCCCCCC] |
| 33 | 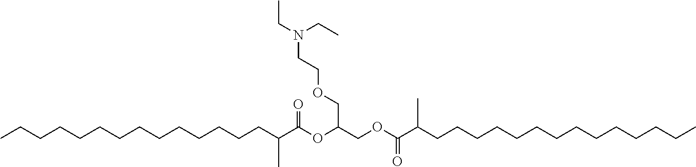 [SMILES: CCCCCCCCCCCCCCC(C)C(=O)OCC(COCCN(CC)CC)OC(=O)C(C)CCCCCCCCCCCCCC] |
| 34 | 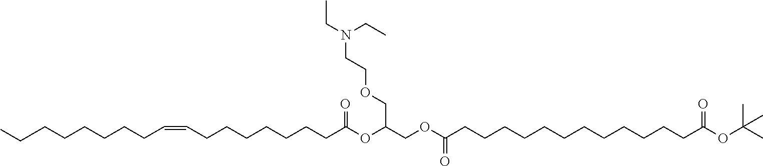 [SMILES: CCCCCCCC/C=C\CCCCCCCC(=O)OC(COCCN(CC)CC)COC(=O)CCCCCCCCCCCCC(=O)OC(C)(C)C] |

TABLE 2-11

| No | structure |
|---|---|
| 35 | 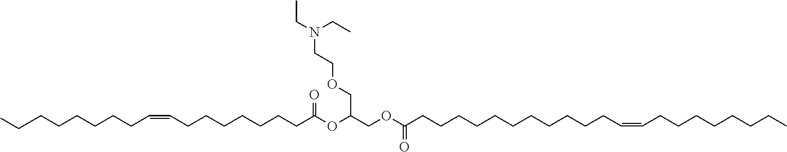 [SMILES: CCCCCCCC/C=C\CCCCCCCCCCCC(=O)OCC(COCCN(CC)CC)OC(=O)CCCCCCC/C=C\CCCCCCCC] |
| 36 | 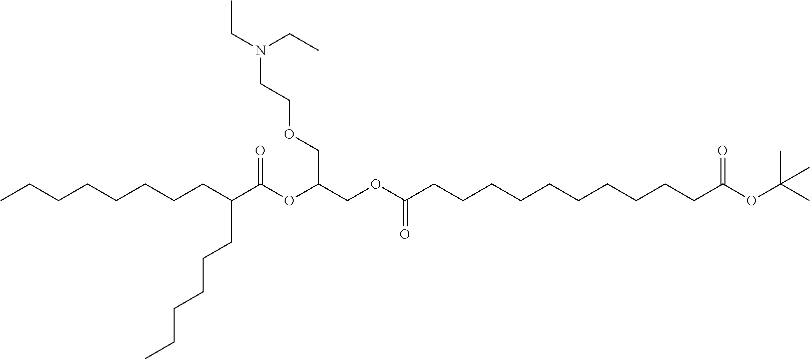 [SMILES: CCCCCCCCC(CCCCCC)C(=O)OC(COCCN(CC)CC)COC(=O)CCCCCCCCCCC(=O)OC(C)(C)C] |
| 37 | 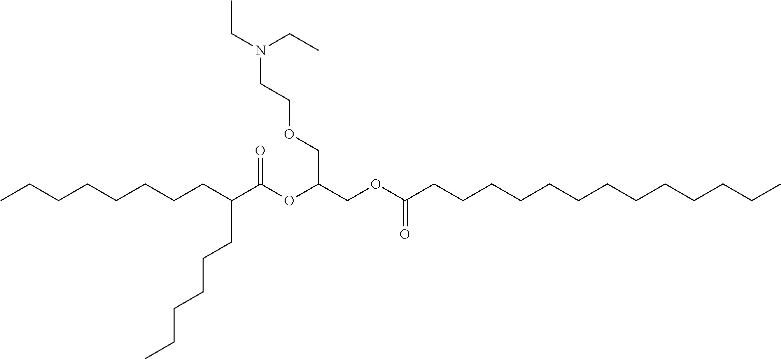 [SMILES: CCCCCCCCCCCCCC(=O)OCC(COCCN(CC)CC)OC(=O)C(CCCCCC)CCCCCCCC] |

The production method of cationic lipid (2A) is now explained.

As shown in Synthetic Scheme 1 below, cationic lipid (2A) can be produced by O-acylation reaction of a glycerol derivative and a fatty acid, but not limited to.

In Synthetic Scheme 1, R¹ to R⁴ are each as defined in the aforementioned formula (2A). TsCl is p-toluenesulfonyl chloride, TBSCl is tert-butyldimethylchlorosilane, DMAP is 4-dimethylaminopyridine, and TBAF is tetrabutylammonium fluoride.

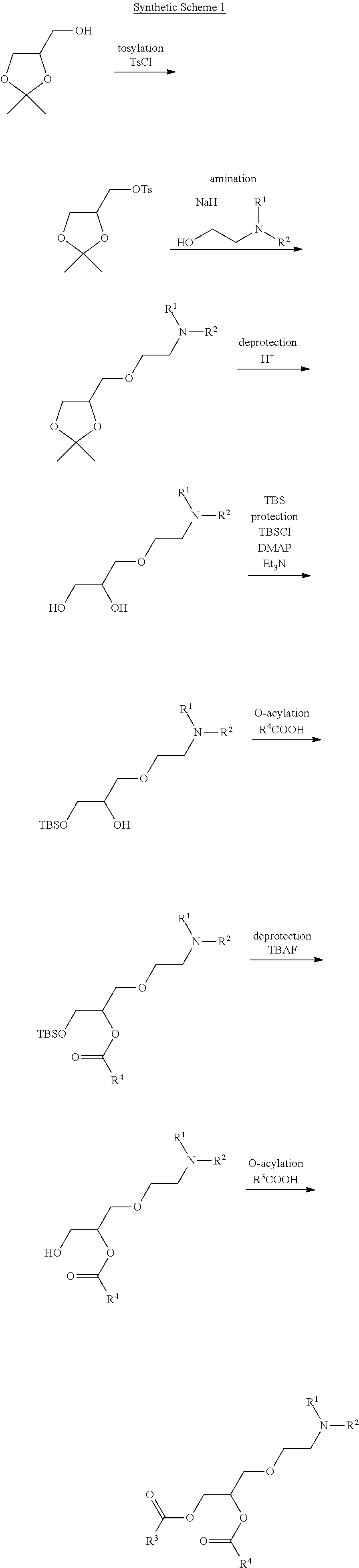

In Synthetic Scheme 1, when R³ and R⁴ are the same aliphatic hydrocarbon group, O-acylation reaction can be performed without TBS protection reaction. Examples of fatty acids that can introduce a aliphatic hydrocarbon group having 1 to 35 carbon atoms into R³ and R⁴ in the amino lipid of one embodiment of the present invention include a saturated fatty acid having 5 to 35 carbon atoms such as lauric acid, myristic acid, palmitic acid, stearic acid, arachidic acid, behenic acid, lignoceric acid, 4-methyl-n-octanoic acid, nonanoic acid, isononanoic acid, monoethyl pimelate, 2,2-dimethylhexanoic acid, caprylic acid, enanthic acid, 2,2-dimethylpentanoic acid, 5-methylhexanoic acid, 3-methylpentanoic acid, 3,3-dimethylbutyric acid, hexanoic acid, 2,2-dimethylbutyric acid, 4-methylpentanoic acid, melissic acid, octacosanoic acid, cerotic acid, 18-(tert-butoxy)-18-oxooctadecanoic acid, nonadacanoic acid, 14-(tert-butoxy)-14-oxotetradecanoic acid, 12-(tert-butoxy)-12-oxododecanoic acid, monoethyl dodecanedioate, undecanoic acid, and the like; a monounsaturated fatty acid having 5 to 35 carbon atoms such as palmitoleic acid, oleic acid, 8-nonenoic acid, erucic acid, elaidic acid, (E)-dodec-2-enoic acid, 10-undecenoic acid, 10-undecynoic acid, trans-2-octenoic acid, 2-heptynoic acid, 2,2-dimethyl-4-pentenoic acid, 2-methylhexenoic acid, 4-methyl-2-pentenoic acid, trans-2-hexenoic acid, 2-methyl-4-pentenoic acid, trans-3-hexenoic acid, 5-hexenoic acid, trans-2-methyl-2-pentenoic acid, 2-methyl-2-pentenoic acid, 5-hexynoic acid, monomethyl itaconate, and the like; a polyunsaturated fatty acid having 5 to 35 carbon atoms such as linoleic acid, γ-linolenic acid, α-linolenic acid, eicosapentaenoic acid, docosahexaenoic acid, 10,12-nonacosadiynoic acid, 10,12-heptacosadiynoic acid, 10,12-pentacosadiynoic acid, arachidonic acid, 2,4-heptadecadiynoic acid, 10,12-heptadecadiynoic acid, sorbic acid, and the like; and a fatty acid having 3 to 35 carbon atoms such as decanoic acid, 9-decenoic acid, 4-methylnonanoic acid, citronellic acid, mono-tert-butyl succinate, 3-(dodecylthio)propionic acid, monomethyl sebacate, monoethyl glutarate, monoethyl itaconate, monoisopropyl fumarate, 3-allyloxypropionic acid, butoxyacetic acid, monoethyl maleate, butyric acid, crotonic acid, pentanoic acid, DL-2-methylbutyric acid, trans-2-pentenoic acid, 3-methoxypropanoic acid, 4-methoxybutanoic acid, monomethyl maleate, monomethyl succinate, 3-ethoxypropionic acid, propiolic acid, 3-methylbutyric acid, 2-methyl-3-butenoic acid, 3-methylcrotonic acid, tetrolic acid, 4-pentenoic acid, methoxyacetic acid, ethoxyacetic acid, 3-(methylthio)propionic acid, pivalic acid, propionic acid, isobutyric acid, 3-(laurylthio)propionic acid, tiglic acid, and the like. Examples of fatty acids that can introduce a secondary alkyl group into R³ and R⁴ include isopalmitic acid, isostearic acid, 2-hexadecyloctadecanoic acid, 2-methylhexadecanoic acid, 2-hexyl-4-pentynoic acid, 2-ethylhexanoic acid, 2-methylheptanoic acid, 2-ethylbutyric acid, and 2-methylpentanoic acid, 2-methylpalmitic acid, and the like.

In Synthetic Scheme 1, examples of amino alcohols that can be used in amination reaction include 2-(dimethylamino)ethanol, 2-(diethylamino)ethanol, 2-(dipropylamino)ethanol, 2-(diisopropylamino)ethanol, 2-(dibutylamino)ethanol, and 2-(dipentylamino)ethanol, 2-(dihexylamino)ethanol, and the like.

In Synthetic Scheme 1, the compounds obtained at each synthesis step can be purified as necessary by recrystallization, reprecipitation, liquid extraction, dialysis, or chromatography.

### Phospholipid

Phospholipids can be used as a lipid membrane constituting component of lipid nanoparticles.

Examples of the phospholipid include 1,2-diacyl-sn-glycero-3-phosphocholine (PC), 1,2-diacyl-sn-glycero-3-phosphatidylethanolamine (PE), 1,2-diacyl-sn-glycero-3-phosphatidylserine (PS), 1,2-diacyl-sn-glycero-3-phosphatidylglycerol (PG), 1,2-diacyl-sn-glycero-3-phosphatidic acid (PA), and lyso forms of these, specifically,
1,2-didecanoyl-sn-glycero-3-phosphocholine (DDPC),
1,2-dilauroyl-sn-glycero-3-phosphocholine (DLPC),
1,2-dimyristoyl-sn-glycero-3-phosphocholine (DMPC),
1,2-dipalmitoyl-sn-glycero-3-phosphocholine (DPPC),
1,2-distearoyl-sn-glycero-3-phosphocholine (DSPC),
1,2-dioleoyl-sn-glycero-3-phosphocholine (DOPC),
1,2-dilinoleoyl-sn-glycero-3-phosphocholine (DLoPC),
1,2-dierucoyl-sn-glycero-3-phosphocholine (DEPC),
1-myristoyl-2-palmitoyl-sn-glycero-3-phosphocholine (MPPC),
1-myristoyl-2-stearoyl-sn-glycero-3-phosphocholine (MSPC),
1-palmitoyl-2-myristoyl-sn-glycero-3-phosphocholine (PMPC),
1-palmitoyl-2-stearoyl-sn-glycero-3-phosphocholine (PSPC),
1-palmitoyl-2-oleoyl-sn-glycero-3-phosphocholine (POPC),
1-stearoyl-2-oleoyl-sn-glycero-3-phosphocholine (SOPC), and these PCs appropriately converted to PE, PS, PG, or PA can be used.

Phospholipid to be used in the present invention is preferably PC or PE, further preferably DOPC, POPC, DOPE (1,2-dioleoyl-sn-glycero-3-phosphoethanolamine), or POPE (1-palmitoyl-2-oleoyl-sn-glycero-3-phosphoethanolamine).

### Sterol

Sterol can be used as a component that regulates fluidity of the lipid membrane of lipid nanoparticles. Examples of the sterol include cholesterol, lanosterol, phytosterol, zymosterol, zymostenol, desmosterol, stigmastanol, dihydrolanosterol, and 7-dehydrocholesterol, preferably cholesterol, lanosterol, and phytosterol, further preferably cholesterol.

### PEG Lipid

PEG lipids are used as stabilizers that coat the surface of lipid nanoparticles with hydrophilic polyethylene glycol (PEG) and suppress the aggregation of particles, or used to suppress the interaction between biological components and particles when administered to a living body.

The PEG region can have any molecular weight. In some embodiments, the PEG region has a molecular weight of 200-10,000 Da and may be linear or branched.

Examples of the PEG lipid include PEG-phospholipid, PEG-ceramide, PEG-diacylglycerol, and PEG-cholesterol, preferably diacylglycerol PEG with a PEG molecular weight of 1,000-10,000, further preferably dimyristoylglycerol PEG or distearoylglycerol PEG with a PEG molecular weight of 1,000-10,000.

### Acidic Buffer and Acidic Buffer Component

A buffer or a buffer component having a buffering action in the acidic region can be used. The “acidic buffer component” means a component obtained by substantially removing water from the acidic buffer solution. The level of water contained in the acidic buffer component may be less than about 5 w/v %, less than 4% w/v, less than 3 w/v %, less than 2 w/v %, or less than 1% w/v. Specifically, HCl/KCl buffer, p-toluenesulfonic acid/sodium p-toluenesulfonate buffer, tartaric acid/NaOH buffer, citric acid/NaOH buffer, phthalic acid HK/HCl buffer, glycine/HCl buffer, trans-aconitic acid/NaOH buffer, formic acid/sodium formate buffer, citric acid/sodium citrate buffer, 3,3-dimethylglutaric acid/NaOH buffer, 3,3-dimethylglutaric acid/NaOH/0.1M NaCl buffer, phenylacetic acid/sodium phenylacetate buffer, acetic acid/sodium acetate buffer, succinic acid/NaOH buffer, phthalic acid HK/NaOH buffer, sodium cacodylate/HCl buffer, maleic acid HNa/NaOH buffer, maleic acid/Tris/NaOH buffer, phosphate buffer, KH₂PO₄/NaOH buffer, imidazole/HCl buffer, s-collidine (2,4,6-trimethylpyridine)/HCl buffer, triethanolamine HCl/NaOH buffer, sodium 5,5-diethylbarbiturate/HCl buffer, N-methylmorpholine/HCl buffer, sodium pyrophosphate/HCl buffer, MES buffer, malic acid buffer, ADA buffer, PIPES buffer, ACES buffer, HEPES, BES, Bis-Tris buffer, Bis-Tris propane buffer, anhydrous sodium carbonate buffer, glycylglycine buffer, MOPS, MOPSO, and TES can be mentioned.

It is preferably an acid buffer or an acid buffer component having a buffering action at pH 1 to 6, more preferably pH 3 to 6. Examples thereof include tartaric acid/NaOH buffer, citric acid/NaOH buffer, phthalic acid HK/HCl buffer, glycine/HCl buffer, trans-aconitic acid/NaOH buffer, formic acid/sodium formate buffer, citric acid/sodium citrate buffer, 3,3-dimethylglutaric acid/NaOH buffer, 3,3-dimethylglutaric acid/NaOH/0.1M NaCl buffer, phenylacetic acid/sodium phenylacetate buffer, acetic acid/sodium acetate buffer, succinic acid/NaOH buffer, phthalic acid HK/NaOH buffer, sodium cacodylate/HCl buffer, maleic acid HNa/NaOH buffer, maleic acid/Tris/NaOH buffer, phosphate buffer, KH₂PO₄/NaOH buffer, MES buffer, malic acid buffer, Bis-Tris buffer, glycylglycine buffer and the like, further preferably malic acid buffer or MES buffer.

The lyophilized composition of the lipid nanoparticles of the present invention preferably has a pH of 1-6, more preferably 3-6, when 100-500 mg of the lyophilized composition is suspended in 1-5 mL of distilled water for injection at 0-30° C.

### Preparation Method of Lipid Nanoparticles

Examples of the “operation to induce organization” for preparing lipid nanoparticles include methods known per se such as an alcohol dilution method using a micro flow path or vortex, a simple hydration method, sonication, heating, vortex, an ether injecting method, a French press method, a cholic acid method, a Ca²⁺ fusion method, a freeze-thaw method, a reversed-phase evaporation method and the like, preferably an alcohol dilution method using a micro flow path or vortex, further preferably an alcohol dilution method using a micro flow path. Preparation of particles by the alcohol dilution method using a micro flow path can be performed using, for example, NanoAssemblr (Precision NanoSystems). The buffer in the external aqueous phase of the prepared lipid nanoparticles can be replaced by an operation such as ultrafiltration, dialysis, or dilution.

### Cryoprotectant

As the cryoprotectant that can be used in the present invention, monosaccharide, sugar alcohol, disaccharide, oligosaccharide, polysaccharide, or polymer can be used. Specific examples include glyceraldehyde, erythrose, threose, ribose, lyxose, xylose, arabinose, allose, talose, gulose, glucose, altrose, mannose, galactose, idose, erythrulose, ribulose, psicose, fructose, sorbose, tagatose, erythritol, glycerol, isomaltol, lactitol, maltitol, mannitol, sorbitol, xylitol, inositol, sucrose, lactulose, lactose, maltose, trehalose, cellobiose, kojibiose, nigerose, isomaltose, isotrehalose, neotrehalose, sophorose, laminaribiose, gentiobiose, turanose, maltulose, palatinose, gentiobiose, mannobiose, melibiose, melibiulose, neolactose, galactosucrose, scillabiose, neohesperidose, rutinose, rutinulose, vicianose, xylobiose, primeverose, trehalosamine, maltitol, lactosamine, lactitol, sucralose, raffinose, panose, maltotriose, melezitose, gentianose, stachyose, cyclodextrin, hydroxypropyl-β-cyclodextrin, dextran, polyvinylpyrrolidone, polyethylene glycol and the like, preferably disaccharide, further preferably non-reducing disaccharide, most preferably sucrose.

The concentration of the cryoprotectant is preferably 80-800 mg/mL, more preferably 160-800 mg/mL, as the concentration before lyophilizing. As the weight ratio of the cryoprotectant to the total lipid after lyophilizing, the weight of the cryoprotectant is preferably 10 to 1000 times, more preferably 30 to 1000 times, that of the total lipid.

### Lyophilization Step

The lyophilization process can be performed in any suitable container known in the technical field of medicine, such as a glass container (or, for example, glass vial) or a two-chamber container.

The stabilized lipid nanoparticle composition of the present invention containing a cryoprotectant can be introduced into a glass container. The volume of the composition to be added to the container may be 0.1-20 mL, or 1-10 mL.

Any lyophilization process (including those known in the technical field of medicine) can be used. See, for example, Remington's Pharmaceutical Sciences, 18th Ed., Mack Publishing Co., Easton, Penn. (1990).

The lyophilization process may include freezing the lipid nanoparticle composition, stabilized by the cryoprotectant, at a temperature of about −55° C. to about −30° C. The frozen composition may be a dried form of the freeze-dried composition.

In some embodiments, the freezing step may include gradually raising the temperature from room temperature to the final temperature over several minutes. The temperature gradient can be about 1° C./min.

In some embodiments, the drying step can be performed at a pressure of about 0-250 mTorr, or 50-150 mTorr, at a temperature of about −55° C. to about 40° C. The drying step can be continued in a high temperature range up to room temperature for a predetermined period of up to several days. The level of residual water in the solid lyophilized composition may be less than about 5 w/v %, or less than 4% w/v, or less than 3 w/v %, or less than 2 w/v %, or less than 1% w/v.

### Rehydration Step

The rehydration step is a step of adding an aqueous solution containing nucleic acid to the lyophilized composition of the present invention to prepare nucleic acid-encapsulating lipid nanoparticles. As for the amount of nucleic acid, an aqueous solution containing nucleic acid such that the ratio of total lipid and nucleic acid contained in the lyophilized composition is, for example, total lipid/nucleic acid=1-1000 nmol/μg, preferably total lipid/nucleic acid=50-500 nmol/μg, is added, and nucleic acid-encapsulating lipid nanoparticles can be prepared by mixing them by pipetting or vortex. In addition, alcohol can be added during rehydration in order to increase the nucleic acid encapsulation rate, and methanol, ethanol, n-butanol and t-butanol, preferably ethanol, can be used as the alcohol. The concentration of alcohol in the aqueous solution containing nucleic acid is 0-50 v/v %, preferably 0-30 v/v %, more preferably 0-25 v/v %. Furthermore, in order to increase the efficiency of nucleic acid encapsulation in the rehydration step, an aqueous solution containing nucleic acid or further alcohol can be added to the lyophilized composition before incubation. The incubation conditions are, for example, 0-100° C. for 0-120 min, preferably 0-95° C. for 0-60 min.

Step of Exchanging External Aqueous Phase with Neutral Buffer

Nucleic acid-encapsulating lipid nanoparticles that can be used as a nucleic acid-introducing agent can be prepared by exchanging the external aqueous phase of the nucleic acid-encapsulating lipid nanoparticles obtained in the rehydration step with a neutral buffer.

Examples of the method of exchanging the external aqueous phase with a neutral buffer include methods using dialysis, ultrafiltration or dilution.

Examples of the neutral buffer include phosphate buffered saline (PBS), Tris-HCl buffer, ADA, PIPES, PIPES sesquisodium, ACES, MOPS, BES, MOPSO, BES, MOPS, TES, HEPES, TAPSO, POPSO, HEPSO and the like. The pH of the neutral buffer is 6-8.

A nucleic acid can be introduced into a cell in vivo and/or in vitro by encapsulating the nucleic acid in the lyophilized composition of the lipid nanoparticles of the present invention and contacting the lipid nanoparticles with the cell. Therefore, the present invention provides a nucleic acid-introducing agent containing the above-mentioned lyophilized composition of the lipid nanoparticles of the present invention.

In addition, the present invention provides a nucleic acid-introducing agent containing nucleic acid-encapsulating lipid nanoparticles prepared by using the lyophilized composition of the lipid nanoparticles of the present invention.

The nucleic acid-introducing agent of the present invention can introduce any nucleic acid into a cell. Examples of the kind of nucleic acid include, but are not limited to, DNA, RNA, chimera nucleic acid of RNA, DNA/RNA hybrid and the like. While any nucleic acid having 1 to 3 chains can be used, it is preferably a single strand or double strand. The nucleic acid may be other type of nucleotide such as N-glycoside of purine or pyrimidine base or other oligomer having a non-nucleotide backbone (e.g., commercially available peptide nucleic acid (PNA) etc.), other oligomer containing a special bond (said oligomer comprising base pairing or a nucleotide having a configuration permitting attachment of base, which are found in DNA and RNA) and the like. Furthermore, it may be a nucleic acid added with known modification, for example, a nucleic acid with a label known in the field, a nucleic acid with a cap, a methylated nucleic acid, one or more natural nucleotides substituted by an analog, a nucleic acid with intramolecular nucleotidyl modification, for example, a nucleic acid with non-charge bond (e.g., methylphosphonate, phosphotriester, phosphoramidate, carbamate and the like), a nucleic acid with a charged bond or sulfur-containing bond (e.g., phosphorothioate, phosphorodithioate and the like), for example, a nucleic acid with a side chain group such as protein (nuclease, nuclease inhibitor, toxin, antibody, signal peptide, poly-L-lysine and the like), sugar (e.g., monosaccharide and the like) and the like, a nucleic acid with an intercalating compound (e.g., acridine, psoralen and the like), a nucleic acid with a chelate compound (e.g., metal, radioactive metal, boron, oxidative metal and the like), a nucleic acid containing an alkylating agent, or a nucleic acid with a modified bond (e.g., a anomer-type nucleic acid and the like).

The type of the DNA that can be used in the present invention is not particularly limited, and can be selected as appropriate according to the purpose of use. For example, plasmid DNA, cDNA, antisense DNA, chromosomal DNA, PAC, BAC, CpG oligosaccharide, and the like can be mentioned. Preferred are plasmid DNA, cDNA and antisense DNA, and more preferred is plasmid DNA. A circular DNA such as plasmid DNA and the like can be digested as appropriate with a restriction enzyme and the like, and also used as a linear DNA.

The type of the RNA that can be used in the present invention is not particularly limited, and can be selected as appropriate according to the purpose of use. For example, siRNA, miRNA, shRNA, antisense RNA, messenger RNA (mRNA), single strand RNA genome, double strand RNA genome, RNA replicon, transfer RNA, ribosomal RNA and the like can be mentioned, with preference given to siRNA, miRNA, shRNA, mRNA, antisense RNA, and RNA replicon.

The nucleic acid used in the present invention is preferably purified by a method generally used by those of ordinary skill in the art.

The nucleic acid-introducing agent of the present invention encapsulating a nucleic acid can be administered in vivo for the purpose of, for example, prevention and/or treatment of diseases. Therefore, the nucleic acid to be used in the present invention is preferably one having a preventive and/or therapeutic activity against a given disease (prophylactic/therapeutic nucleic acid). Examples of such nucleic acid include nucleic acids and the like used for so-called gene therapy.

The nucleic acid-introducing agent of the present invention encapsulating a nucleic acid can be used as a drug delivery system for selectively delivering a nucleic acid and the like into a particular cell, and is useful for, for example, DNA vaccines by introducing antigen gene into dendritic cells, gene therapy drugs for tumor, nucleic acid pharmaceutical products that suppress expression of target genes by utilizing RNA interference, and the like.

The particle size of the lipid nanoparticles encapsulating the nucleic acid is not particularly limited, and is preferably 10 nm-500 nm, more preferably 30 nm-300 nm. The particle size can be measured by using a particle size distribution measuring device such as Zetasizer Nano (Malvern) or the like. The particle size of the lipid nanoparticles can be appropriately adjusted by the method for preparing the lipid membrane structure.

The surface charge (zeta potential) of the lipid nanoparticles encapsulating the nucleic acid is not particularly limited and preferably −15 to +15 mV, more preferably −10 to +10 mV. In conventional transgene, particles electrically charged to have a plus surface potential have been mainly used. This is useful as a method for promoting electrostatic interactions with heparin sulfate on the negatively-charged cell surface to enhance uptake into cells. However, the positive surface charge may suppress, in the cell, release of nucleic acid from the carrier due to the interaction with a nucleic acid to be delivered or protein synthesis due to the interaction between mRNA and a nucleic acid to be delivered. This problem can be solved by adjusting the surface charge to fall within the above-mentioned range. The surface charge can be measured using a zeta potential measuring apparatus such as Zetasizer Nano and the like. The surface charge of the lipid nanoparticles can be adjusted by the composition of the constituent component of the lipid nanoparticles.

The step of contacting the lipid nanoparticles encapsulating the nucleic acid with the cell in vitro is specifically explained below.

The cells are suspended in a suitable medium several days before contact with the lipid nanoparticles, and cultured under appropriate conditions. At the time of contact with the lipid nanoparticles, the cells may or may not be in a proliferative phase.

The culture medium on contact may be a serum-containing medium or a serum-free medium, wherein the serum concentration of the medium is preferably not more than 30 wt %, more preferably not more than 20 wt %, since when the medium contains excess protein such as serum and the like, the contact between the lipid nanoparticles and the cell may be inhibited.

The cell density on contact is not particularly limited, and can be appropriately determined in consideration of the kind of the cell and the like. It is generally within the range of 1×10⁴-1×10⁷ cells/mL.

A suspension of the aforementioned lipid nanoparticles encapsulating the nucleic acid is added to the thus-prepared cells. The amount of the suspension to be added is not particularly limited, and can be appropriately determined in consideration of the cell number and the like. The concentration of the lipid nanoparticles to be contacted with the cells is not particularly limited as long as the desired introduction of the nucleic acid into the cells can be achieved. The lipid concentration is generally 1-100 nmol/ml, preferably 10-50 nmol/ml, and the concentration of the nucleic acid is generally 0.01-100 μg/ml, preferably 0.1-10 μg/ml.

After the aforementioned suspension is added to cells, the cells are cultured. The temperature, humidity and CO₂ concentration during culturing are appropriately determined in consideration of the kind of the cell. When the cell is derived from a mammal, generally, the temperature is about 37° C., humidity is about 95% and CO₂ concentration is about 5%. While the culture period can also be appropriately determined in consideration of the conditions such as the kind of the cell and the like, it is generally a range of 0.1-76 hr, preferably a range of 0.2-24 hr, more preferably a range of 0.5-12 hr. When the above-mentioned culture time is too short, the nucleic acid is not sufficiently introduced into the cells, and when the culture time is too long, the cells may become weak.

By the above-mentioned culture, the nucleic acid is introduced into cells. The culture is further continued preferably by exchanging the medium with a fresh medium, or adding a fresh medium to the medium. When the cell is a mammal-derived cell, the fresh medium preferably contains a serum or nutrition factor.

As mentioned above, a nucleic acid can be introduced into cells not only outside the body (in vitro) but also in the body (in vivo) by using lipid nanoparticles encapsulating the nucleic acid. That is, by administration of the lipid nanoparticles encapsulating the nucleic acid to a subject, the lipid nanoparticles reaches and contacts with the target cells, and the nucleic acid encapsulated in the lipid nanoparticles is introduced into the cells in vivo. The subject to which the lipid nanoparticles can be administered is not particularly limited and, for example, vertebrates such as mammals (e.g., human, monkey, mouse, rat, hamster, bovine etc.), birds (e.g., chicken, ostrich etc.), amphibia (e.g., frog etc.), fishes (e.g., zebrafish, rice-fish etc.) and the like, invertebrates such as insects (e.g., silk moth, moth, Drosophila etc.) and the like, plants and the like can be mentioned. The subject of administration of the lipid nanoparticles encapsulating the nucleic acid is preferably human or other mammal.

The kind of the target cell is not particularly limited, and a nucleic acid can be introduced into cells in various tissues (e.g., liver, kidney, pancreas, lung, spleen, heart, blood, muscle, bone, brain, stomach, small intestine, large intestine, skin, adipose tissue, lymph node, tumor, etc.) by using the lipid nanoparticles encapsulating the nucleic acid.

The administration method of the lipid nanoparticles into which a nucleic acid and/or a compound other than nucleic acid is introduced to a target (e.g., vertebrate, invertebrate and the like) is not particularly limited as long as the lipid nanoparticles reaches and contacts with the target cells, and the compound introduced into the lipid nanoparticles can be introduced into the cell, and an administration method known per se (e.g., oral administration, parenteral administration (e.g., intravenous administration, intramuscular administration, topical administration, transdermal administration, subcutaneous administration, intraperitoneal administration, spray etc.) etc.) can be appropriately selected in consideration of the kind of the compound to be introduced, the kind and the site of the target cell and the like. The dose of the lipid nanoparticles is not particularly limited as long as the introduction of the compound into the cells can be achieved, and can be appropriately selected in consideration of the kind of the subject of administration, administration method, the kind of the compound to be introduced, the kind and the site of the target cell and the like.

When the lipid nanoparticles encapsulating the nucleic acid are used as a nucleic acid-introducing agent, they can be formulated according to a conventional method.

When the nucleic acid-introducing agent is provided as a reagent for studies, the lipid nanoparticles encapsulating the nucleic acid may be provided as it is as the nucleic acid-introducing agent of the present invention, or the nucleic acid-introducing agent of the present invention may be provided as a sterile solution or suspension with, for example, water or other physiologically acceptable liquid (e.g., water-soluble solvent (e.g., malic acid buffer etc.), organic solvent (e.g., ethanol, methanol, DMSO, tert-butanol and the like), or a mixture of aqueous solvent and organic solvent etc.). The nucleic acid-introducing agent of the present invention may appropriately contain physiologically acceptable additive (e.g., excipient, vehicle, preservative, stabilizer, binder etc.), which are known per se.

When the nucleic acid-introducing agent is provide as a medicament, the lipid nanoparticles encapsulating the nucleic acid may be used as it is as the nucleic acid-introducing agent of the present invention or the nucleic acid-introducing agent of the present invention may be produced as an oral preparation (for example, tablet, capsule etc.) or a parenteral agent (for example, injection, spray etc.), preferably a parenteral agent (more preferably, injection) by blending with a pharmaceutically acceptable known additives such as carrier, flavor, excipient, vehicle, preservative, stabilizer, binder and the like in a conventionally-admitted unit dosage form required for practicing preparation formulation.

The nucleic acid-introducing agent of the present invention can be formulated into a preparation not only for adults but also for children.

### EXAMPLES

The Examples of the present invention are explained in further detail in the following, but the present invention is not limited in any way by the Examples.

The abbreviations used in the present specification each mean the following.
Chol: cholesterol
DMG-PEG2k: 1,2-dimyristoyl-rac-glycerol, methoxypolyethylene glycol (PEG MW 2000)
DSPC: 1,2-distearoyl-sn-glycero-3-phosphocholine
DOPC: 1,2-dioleoyl-sn-glycero-3-phosphocholine
POPC: 1-palmitoyl-2-oleoyl-sn-glycero-3-phosphocholine
DOPE: 1,2-dioleoyl-sn-glycero-3-phosphoethanolamine
POPE: 1-palmitoyl-2-oleoyl-sn-glycero-3-phosphoethanolamine
PBS: phosphate buffered saline
MES: 2-morpholinoethanesulfonic acid
DPBS: Dulbecco's phosphate buffered saline

### Preparation of Lyophilized Compositions of Comparative Examples 1-3

As Comparative Examples, neutral lipid nanoparticles were produced as described below, a cryoprotectant was added, and the mixture was lyophilized to prepare lyophilized compositions.

According to the lipid compositions shown in Table 4, a tert-butanol solution of a lipid and malic acid buffer (pH 3, 20 mM) were mixed by NanoAssemblr (flow rate ratio: buffer/lipid=6/1(v/v), total flow rate: 1 mL/min). This solution was diluted with MES buffer (pH 5.5, 20 mM), and replaced with PBS at pH 7.4 while performing ultrafiltration using Amicon. LNP was concentrated such that the lipid concentration was 200 nmol/100 μL as a theoretical value and recovered, and 100 μL of a sucrose solution was added thereto and mixed. This solution was freeze-dried using a VerTis AdVantage Plus EL-85 freeze-dryer. For lyophilizing, the solution was first frozen at normal pressure at −55° C. for 14 to 21 hr, then the pressure was reduced to 200 mTorr and the temperature was raised by 10° C. The temperature rise program was set to raise the temperature by 10° C. over 3 hr up to −20° C., raise the temperature by 10° C. over 2 hr above −10° C., and allow the sample to stand at said temperature for 1 hr. When the temperature rose to 30° C., the sample was allowed to stand at said temperature for 3 hr, the pressure was returned to normal pressure, and the sample was collected.

### Preparation of Lyophilized Compositions of Examples 1-59

According to the lipid compositions shown in Table 5, a tert-butanol solution of a lipid and malic acid buffer (pH 3.0, 20 mM) were mixed by NanoAssemblr (flow rate ratio: buffer/lipid=6/1(v/v), total flow rate: 1 mL/min). This LNP solution was mixed with an equal amount of a sucrose solution (such that the sucrose final concentration was 80 mg/mL, 160 mg/mL, 320 mg/mL, or 433 mg/mL), and dispensed to a lipid amount of 200 nmol or 600 nmol. This solution was freeze-dried using a VerTis AdVantage Plus EL-85 freeze-dryer. For lyophilizing, the solution was first frozen at normal pressure at −55° C. for 14 to 21 hr, then the pressure was reduced to 200 mTorr and the temperature was raised by 10° C. The temperature rise program was set to raise the temperature by 10° C. over 3 hr up to −20° C., raise the temperature by 10° C. over 2 hr above −10° C., and allow the sample to stand at said temperature for 1 hr. When the temperature rose to 30° C., the sample was allowed to stand at said temperature for 3 hr, the pressure was returned to normal pressure, and the sample was collected.

### Preparation of Lyophilized Compositions of Examples 60-70

According to the lipid compositions shown in Table 5, a tert-butanol solution of a lipid and MES buffer (pH 5.0, 20 mM) were mixed by NanoAssemblr (flow rate ratio: buffer/lipid=6/1(v/v), total flow rate: 1 mL/min). Subsequent operations were performed in the same manner as in Examples 1-59.

### Rehydration of Lyophilized Compositions of Comparative Examples 1-3 and Examples 1-59

Under the conditions shown in Tables 4 and 5, an aqueous solution of mRNA, siRNA, or pDNA (RNase-free, containing 12.5% or 25% EtOH) was added to a lyophilized composition at a ratio of total lipid/mRNA (or siRNA or pDNA)=200 nmol/μg, and mixed by pipetting. The mixture was diluted with MES buffer (pH 5.5, 20 mM), and replaced with PBS at pH 7.4 while performing ultrafiltration using Amicon. LNP was concentrated such that the mRNA (or siRNA or pDNA) concentration was 2.5 g/mL as a theoretical value and recovered. Using Ribogreen (registered trade mark) reagent or Picogreen (registered trade mark) reagent, the recovery rate and the encapsulation rate of the nucleic acid were measured. The particle size and zeta potential were measured using Zetasizer.

### Measurement of Recovery Rate and Encapsulation Rate

(1) A solution (250 μL) is prepared by diluting a nucleic acid-encapsulating LNP solution 2.5-fold with DPBS (theoretical value 1 μg/mL).
(2) For calibration curve, a solution (250 μL each) is prepared by diluting an mRNA solution of known concentration with DPBS to an mRNA concentration of 2000 ng/mL, 1000 ng/mL, 500 ng/mL, 250 ng/mL, 125 ng/mL, or 0 ng/mL.
(3) RiboGreen reagent or PicoGreen reagent is prepared under the conditions shown in the Table below (amount per well). An amount of (calibration curve number+sample number+1)×2 is prepared for analysis at n=2.

TABLE 3

| | SOLUTION | SOLUTION |
|---|---|---|
| | A (μL) | B (μL) |
| DPBS | 47.75 | 49.75 |
| 10% (w/v) TritonX−100 solution | 2 | 0 |
| (TritonX−100 (Sigma−Aldrich, T8787) | | |
| was diluted with Milli−Q water) | | |
| RiboGreen or Picogreen reagent | 0.25 | 0.25 |
| Total | 50 | 50 |

(4) 50 μL of the solution prepared in (1) and (2) is added to each well of a black 96 well plate, and then add 50 μL of SOLUTION A or 50 μL of SOLUTION B is added.
(5) The plate is shaded with aluminum foil and incubated for 5 min on a shaker (100-500 rpm).
(6) The fluorescence is measured with a microplate reader (Excitation: 480 nm, Emission: 520 nm).
(7) The recovery rate and the encapsulation rate are calculated by the following calculation formulas.

$$recovery rate ( \% ) = \frac{concentration obtained from conditions for SOLUTION A addition}{concentration at 100 \% recovery} \times 100$$

$$encapsulation rate ( \% ) = ( 1 - \frac{concentration obtained from SOLUTION B - added sample}{concentration obtained from conditions for SOLUTION A addition} ) \times 100$$

### Rehydration of Lyophilized Compositions of Examples 60-63

An aqueous solution of mRNA (RNase free) was added to a lyophilized composition at a ratio of total lipid/mRNA=200 nmol/μg, mixed by pipetting, and incubated at 95° C. for 5 min. The mixture was diluted with MES buffer (pH 5.5, 20 mM), and replaced with PBS at pH 7.4 while performing ultrafiltration using Amicon. LNP was concentrated to 2.5 μg/mL as mRNA concentration and recovered. Using Ribogreen reagent, the recovery rate and the encapsulation rate of the nucleic acid were measured. The particle size and zeta potential were measured using Zetasizer.

### Rehydration of Lyophilized Compositions of Examples 64 and 65

An aqueous solution of mRNA (RNase free) was added to a lyophilized composition at a ratio of total lipid/mRNA=200 nmol/μg, mixed by pipetting, and incubated at 95° C. for 5 min. Using Ribogreen reagent, the recovery rate and the encapsulation rate of the nucleic acid were measured. The particle size and zeta potential were measured using Zetasizer.

### Rehydration of Lyophilized Compositions of Examples 66-70

An aqueous solution of mRNA (RNase free) was added to a lyophilized composition at a ratio of total lipid/mRNA=200 nmol/μg, and mixed by pipetting. The mixture was diluted with MES buffer (pH 5.5, 20 mM), and replaced with PBS at pH 7.4 while performing ultrafiltration using Amicon. LNP was concentrated to 2.5 μg/mL as mRNA concentration and recovered. Using Ribogreen reagent, the recovery rate and the encapsulation rate of the nucleic acid were measured. The particle size and zeta potential were measured using Zetasizer.

The mRNA, siRNA, and pDNA sequences used are shown below.
mRNA: CleanCap (registered trade mark) FLuc mRNA (TriLink)
CleanCap (registered trade mark) EGFP mRNA (TriLink)
CleanCap (registered trade mark) EPO mRNA (5moU) (TriLink)
CleanCap (registered trade mark) OVA mRNA (TriLink)
pDNA: pcDNA3.1-luc (the method described in Biomaterials 2011, 32, 6342.)
siRNA: siFVII (2′-F)

Sense
5′-GGAucAucucAAGucuuAcdT*dT-3′
Antisense
5′-GuAAGAcuuGAGAuGAuccdT*dT-3′

dT: deoxythymidine
*: phosphorothioate linkage
Capital letter: native (2′-OH) ribonucleotides
Small letter: 2′-Fluoro-modified nucleotides

TABLE 4
Compositions of lyophilized products of Comparative Examples and rehydration conditions
preparation of neutral lipid nanoparticles, addition of cryoprotectant, lyophilizing, and then rehydration

| | ionic lipid, | sterol, type | PEG lipid, | phospho- | sucrose | | ethanol | type |
|---|---|---|---|---|---|---|---|---|
| | type and | and ratio | type and | lipid, | concen- | weight | concen- | of |
| sample | ratio (molar | (molar | ratio (molar | type and ratio | tration | ratio | tration | nucleic |
| name | ratio) | ratio) | ratio) | (molar ratio) | (mg/mL) | Note 1 | (v/v %) | acid |
| Comparative | SS-OP | Chol | DMG-PEG2k | DOPC | 80 | 90.8 | 0 | mRNA |
| Example 1 | 52.5 | 40 | 1 | 7.5 | | | | |
| Comparative | SS-OP | Chol | DMG-PEG2k | DOPC | 160 | 181.6 | 0 | mRNA |
| Example 2 | 52.5 | 40 | 1 | 7.5 | | | | |
| Comparative | SS-OP | Chol | DMG-PEG2k | DOPC | 320 | 363.3 | 0 | mRNA |
| Example 3 | 52.5 | 40 | 1 | 7.5 | | | | |

Note 1:
weight ratio of sucrose to total lipid

TABLE 5
Compositions of lyophilized products of Examples and rehydration conditions

| | | | PEG lipid, | phospho- | sucrose | | ethanol | |
|---|---|---|---|---|---|---|---|---|
| | ionic lipid, | sterol, type | type and | lipid, type | concen- | weight | concen- | type of |
| sample | type and ratio | and ratio | ratio (molar | and ratio | tration | ratio | tration | nucleic |
| name | (molar ratio) | (molar ratio) | ratio) | (molar ratio) | (mg/mL) | Note 1 | (v/v %) | acid |
| Example | SS-OP | Chol | DMG-PEG2k | DOPC | 80 | 90.8 | 0 | mRNA |
| 1 | 52.5 | 40 | 1 | 7.5 | | | | |
| Example | SS-OP | Chol | DMG-PEG2k | DOPC | 160 | 181.6 | 0 | mRNA |
| 2 | 52.5 | 40 | 1 | 7.5 | | | | |
| Example | SS-OP | Chol | DMG-PEG2k | DOPC | 320 | 363.3 | 0 | mRNA |
| 3 | 52.5 | 40 | 1 | 7.5 | | | | |
| Example | SS-OP | Chol | DMG-PEG2k | DOPC | 160 | 90.8 | 12.5 | mRNA |
| 4 | 52.5 | 40 | 1 | 7.5 | | | | |
| Example | SS-OP | Chol | DMG-PEG2k | DOPC | 160 | 181.6 | 25 | mRNA |
| 5 | 52.5 | 40 | 1 | 7.5 | | | | |
| Example | SS-OP | Chol | DMG-PEG2k | DOPC | 320 | 363.3 | 12.5 | mRNA |
| 6 | 52.5 | 40 | 1 | 7.5 | | | | |
| Example | SS-OP | Chol | DMG-PEG2k | DOPC | 320 | 363.3 | 25 | mRNA |
| 7 | 52.5 | 40 | 1 | 7.5 | | | | |
| Example | SS-OP | Chol | DMG-PEG2k | DOPC | 433 | 491.5 | 0 | mRNA |
| 8 | 52.5 | 40 | 1 | 7.5 | | | | |
| Example | SS-OP | Chol | DMG-PEG2k | DOPC | 320 | 363.3 | 12.5 | pDNA |
| 9 | 52.5 | 40 | 1 | 7.5 | | | | |
| Example | SS-OP | Chol | DMG-PEG2k | none | 320 | 322.7 | 12.5 | siRNA |
| 10 | 70 | 30 | 1 | | | | | |
| Example | SS-OP | Chol | DMG-PEG2k | DOPE | 320 | 377.7 | 12.5 | mRNA |
| 11 | 60 | 30 | 1 | 10 | | | | |
| Example | SS-OP | Chol | DMG-PEG2k | DOPE | 320 | 325.1 | 12.5 | mRNA |
| 12 | 60 | 20 | 1 | 20 | | | | |
| Example | SS-OP | Chol | DMG-PEG2k | DOPE | 320 | 313.4 | 12.5 | mRNA |
| 13 | 60 | 10 | 1 | 30 | | | | |
| Example | SS-OP | Chol | DMG-PEG2k | DOPE | 320 | 407.3 | 12.5 | mRNA |
| 14 | 40 | 50 | 1 | 10 | | | | |
| Example | SS-OP | Chol | DMG-PEG2k | DOPE | 320 | 389.1 | 12.5 | mRNA |
| 15 | 40 | 40 | 1 | 20 | | | | |
| Example | SS-OP | Chol | DMG-PEG2k | DOPE | 320 | 372.5 | 12.5 | mRNA |
| 16 | 40 | 30 | 1 | 30 | | | | |
| Example | SS-OP | Chol | DMG-PEG2k | DOPE | 320 | 357.2 | 12.5 | mRNA |
| 17 | 40 | 20 | 1 | 40 | | | | |
| Example | SS-OP | Chol | DMG-PEG2k | POPE | 320 | 338.7 | 12.5 | mRNA |
| 18 | 60 | 30 | 1 | 10 | | | | |
| Example | SS-OP | Chol | DMG-PEG2k | POPE | 320 | 326.9 | 12.5 | mRNA |
| 19 | 60 | 20 | 1 | 20 | | | | |
| Example | SS-OP | Chol | DMG-PEG2k | POPE | 320 | 315.9 | 12.5 | mRNA |
| 20 | 60 | 10 | 1 | 30 | | | | |
| Example | SS-OP | Chol | DMG-PEG2k | POPE | 320 | 408.7 | 12.5 | mRNA |
| 21 | 40 | 50 | 1 | 10 | | | | |
| Example | SS-OP | Chol | DMG-PEG2k | POPE | 320 | 391.7 | 12.5 | mRNA |
| 22 | 40 | 40 | 1 | 20 | | | | |
| Example | SS-OP | Chol | DMG-PEG2k | POPE | 320 | 376.0 | 12.5 | mRNA |
| 23 | 40 | 30 | 1 | 30 | | | | |
| Example | SS-OP | Chol | DMG-PEG2k | POPE | 320 | 361.5 | 12.5 | mRNA |
| 24 | 40 | 20 | 1 | 40 | | | | |
| Example | SS-OP | Chol | DMG-PEG2k | POPC | 320 | 354.6 | 12.5 | mRNA |
| 25 | 58 | 40 | 1 | 2 | | | | |
| Example | SS-OP | Chol | DMG-PEG2k | POPC | 320 | 356.3 | 12.5 | mRNA |
| 26 | 57 | 40 | 1 | 3 | | | | |
| Example | SS-OP | Chol | DMG-PEG2k | POPC | 320 | 358.0 | 12.5 | mRNA |
| 27 | 56 | 40 | 1 | 4 | | | | |
| Example | SS-OP | Chol | DMG-PEG2k | POPC | 320 | 359.7 | 12.5 | mRNA |
| 28 | 55 | 40 | 1 | 5 | | | | |
| Example | SS-OP | Chol | DMG-PEG2k | POPC | 320 | 361.5 | 12.5 | mRNA |
| 29 | 54 | 40 | 1 | 6 | | | | |
| Example | SS-OP | Chol | DMG-PEG2k | POPC | 320 | 363.2 | 12.5 | mRNA |
| 30 | 53 | 40 | 1 | 7 | | | | |
| Example | SS-OP | Chol | DMG-PEG2k | none | 320 | 351.3 | 0 | mRNA |
| 31 | 60 | 40 | 1 | | | | | |
| Example | SS-OP | Chol | DMG-PEG2k | none | 320 | 351.3 | 25 | mRNA |
| 32 | 60 | 40 | 1 | | | | | |
| Example | SS-OP | Chol | DMG-PEG2k | DOPC | 320 | 359.2 | 0 | mRNA |
| 33 | 55 | 40 | 1 | 5 | | | | |
| Example | SS-OP | Chol | DMG-PEG2k | DOPC | 320 | 362.4 | 0 | mRNA |
| 34 | 53 | 40 | 1 | 7 | | | | |
| Example | SS-OP | Chol | DMG-PEG2k | DOPC | 320 | 359.2 | 25 | mRNA |
| 35 | 55 | 40 | 1 | 5 | | | | |
| Example | SS-OP | Chol | DMG-PEG2k | DOPC | 320 | 367.4 | 0 | mRNA |
| 36 | 50 | 40 | 1 | 10 | | | | |
| Example | SS-OP | Chol | DMG-PEG2k | DOPC | 320 | 376.0 | 0 | mRNA |
| 37 | 45 | 40 | 1 | 15 | | | | |
| Example | SS-OP | Chol | DMG-PEG2k | none | 320 | 351.3 | 12.5 | mRNA |
| 38 | 60 | 40 | 1 | | | | | |
| Example | SS-OP | Chol | DMG-PEG2k | DOPC | 320 | 359.2 | 12.5 | mRNA |
| 39 | 55 | 40 | 1 | 5 | | | | |
| Example | DODMA | Chol | DMG-PEG2k | DOPC | 160 | 275.0 | 0 | pDNA |
| 40 | 52.5 | 40 | 1 | 7.5 | | | | |
| Example | DODMA | Chol | DMG-PEG2k | DOPC | 160 | 275.0 | 12.5 | pDNA |
| 41 | 52.5 | 40 | 1 | 7.5 | | | | |
| Example | DODMA | Chol | DMG-PEG2k | DOPC | 160 | 275.0 | 25 | pDNA |
| 42 | 52.5 | 40 | 1 | 7.5 | | | | |
| Example | DODMA | Chol | DMG-PEG2k | DOPC | 320 | 549.9 | 0 | pDNA |
| 43 | 52.5 | 40 | 1 | 7.5 | | | | |
| Example | DODMA | Chol | DMG-PEG2k | DOPC | 320 | 549.9 | 12.5 | pDNA |
| 44 | 52.5 | 40 | 1 | 7.5 | | | | |
| Example | DODMA | Chol | DMG-PEG2k | DOPC | 320 | 549.9 | 25 | pDNA |
| 45 | 52.5 | 40 | 1 | 7.5 | | | | |
| Example | MC3 | Chol | DMG-PEG2k | DSPC | 160 | 264.2 | 0 | pDNA |
| 46 | 50 | 38.5 | 1.5 | 10 | | | | |
| Example | MC3 | Chol | DMG-PEG2k | DSPC | 160 | 264.2 | 12.5 | pDNA |
| 47 | 50 | 38.5 | 1.5 | 10 | | | | |
| Example | MC3 | Chol | DMG-PEG2k | DSPC | 160 | 264.2 | 25 | pDNA |
| 48 | 50 | 38.5 | 1.5 | 10 | | | | |
| Example | MC3 | Chol | DMG-PEG2k | DSPC | 320 | 528.4 | 0 | pDNA |
| 49 | 50 | 38.5 | 1.5 | 10 | | | | |
| Example | MC3 | Chol | DMG-PEG2k | DSPC | 320 | 528.4 | 12.5 | pDNA |
| 50 | 50 | 38.5 | 1.5 | 10 | | | | |
| Example | MC3 | Chol | DMG-PEG2k | DSPC | 320 | 528.4 | 25 | pDNA |
| 51 | 50 | 38.5 | 1.5 | 10 | | | | |
| Example | SS-OP | Chol | DMG-PEG2k | DOPC | 80 | 90.8 | 12.5 | mRNA |
| 52 | 52.5 | 40 | 1 | 7.5 | | | | |
| Example | SS-OP | Chol | DMG-PEG2k | DOPC | 320 | 354.4 | 12.5 | mRNA |
| 53 | 58 | 40 | 1 | 2 | | | | |
| Example | SS-OP | Chol | DMG-PEG2k | DOPC | 320 | 356.0 | 12.5 | mRNA |
| 54 | 57 | 40 | 1 | 3 | | | | |
| Example | SS-OP | Chol | DMG-PEG2k | DOPC | 320 | 357.6 | 0 | mRNA |
| 55 | 56 | 40 | 1 | 4 | | | | |
| Example | SS-OP | Chol | DMG-PEG2k | DOPC | 320 | 357.6 | 12.5 | mRNA |
| 56 | 56 | 40 | 1 | 4 | | | | |
| Example | SS-OP | Chol | DMG-PEG2k | DOPC | 320 | 360.8 | 0 | mRNA |
| 57 | 54 | 40 | 1 | 6 | | | | |
| Example | SS-OP | Chol | DMG-PEG2k | DOPC | 320 | 360.8 | 12.5 | mRNA |
| 58 | 54 | 40 | 1 | 6 | | | | |
| Example | SS-OP | Chol | DMG-PEG2k | DOPC | 320 | 362.4 | 12.5 | mRNA |
| 59 | 53 | 40 | 1 | 7 | | | | |
| Example | SS-EP | Chol | DMG-PEG2k | DOPE | 160 | 127.6 | 0 | mRNA |
| 60 | 60 | 30 | 1 | 10 | | | | |
| Example | SS-EP | Chol | DMG-PEG2k | DOPC | 160 | 127.2 | 0 | mRNA |
| 61 | 60 | 30 | 1 | 10 | | | | |
| Example | SS-EP | Chol | DMG-PEG2k | DOPE | 160 | 139.6 | 0 | mRNA |
| 62 | 52.5 | 40 | 1 | 7.5 | | | | |
| Example | SS-EP | Chol | DMG-PEG2k | DOPC | 160 | 139.2 | 0 | mRNA |
| 63 | 52.5 | 40 | 1 | 7.5 | | | | |
| Example 64 | SS-EP | Chol | DMG-PEG2k | DOPE | 160 | 127.6 | 0 | mRNA |
| (no ultrafil- | 60 | 30 | 1 | 10 | | | | |
| tration after | | | | | | | | |
| rehydration) | | | | | | | | |
| Example 65 | SS-EP | Chol | DMG-PEG2k | DOPE | 160 | 139.6 | 0 | mRNA |
| (no ultrafil- | 52.5 | 40 | 1 | 7.5 | | | | |
| tration after | | | | | | | | |
| rehydration) | | | | | | | | |
| Example | B-2 | Chol | DMG-PEG2k | DOPC | 320 | 521.1 | 0 | mRNA |
| 66 | 52.5 | 40 | 1 | 7.5 | | | | |
| Example | B-2-5 | Chol | DMG-PEG2k | DOPC | 320 | 342.5 | 0 | mRNA |
| 67 | 52.5 | 40 | 1 | 7.5 | | | | |
| Example | TS-P4C2 | Chol | DMG-PEG2k | DOPC | 320 | 327.4 | 0 | mRNA |
| 68 | 52.5 | 40 | 1 | 7.5 | | | | |
| Example | L-P4C2 | Chol | DMG-PEG2k | DOPC | 320 | 448.5 | 0 | mRNA |
| 69 | 52.5 | 40 | 1 | 7.5 | | | | |
| Example | O-P4C2 | Chol | DMG-PEG2k | DOPC | 320 | 447.2 | 0 | mRNA |
| 70 | 52.5 | 40 | 1 | 7.5 | | | | |

Note 1:
weight ratio of sucrose to total lipid

According to the operations, the lyophilized compositions of neutral lipid nanoparticles of Comparative Examples were rehydrated, and the evaluation results of the particle property, and the recovery rate and encapsulation rate of nucleic acid are shown in Table 6.

As shown in the evaluation results in Table 6, in the freeze-dried compositions of neutral lipid nanoparticles, nucleic acid and ionic lipid do not electrostatically interact with each other during rehydration with an aqueous nucleic acid solution. Therefore, the nucleic acid could not be encapsulated in the lipid nanoparticles.

TABLE 6
Property evaluation results of lipid nanoparticles−1

| | Size | | | | |
|---|---|---|---|---|---|
| | number | | Z | recovery | |
| | average | | potential | rate | encapsulation |
| | (nm) | PDI | (mV) | (%) | rate (%) |
| Comparative | 164.4 | 0.120 | −4.51 | 106.9 | 2.4 |
| Example 1 | | | | | |
| Comparative | 119.3 | 0.113 | −2.14 | 88.7 | 0 |
| Example 2 | | | | | |
| Comparative | 170.7 | 0.130 | −2.88 | 84.4 | 0 |
| Example 3 | | | | | |

According to the operations, the lyophilized compositions of acidic lipid nanoparticles in which the concentration of sucrose as a cryoprotectant was changed were rehydrated, and the evaluation results of the particle property, and the recovery rate and encapsulation rate of nucleic acid are shown in Table 7.

As shown in the evaluation results in Table 7, a nucleic acid could be efficiently encapsulated in the acidic lyophilized compositions of Examples 1-3. Furthermore, as shown in Table 7, the encapsulation efficiency of nucleic acid could be enhanced by increasing the concentration of sucrose as a cryoprotectant, and lipid nanoparticles having a small particle distribution in terms of particle property could be prepared.

TABLE 7
Property evaluation results of lipid nanoparticles−2

| | Size | | | | |
|---|---|---|---|---|---|
| | number | | Z | recovery | |
| | average | | potential | rate | encapsulation |
| | (nm) | PDI | (mV) | (%) | rate (%) |
| Example 1 | 144 | 0.253 | −6.4 | 60 | 56 |
| Example 2 | 105.5 | 0.176 | −5.0 | 79.9 | 72.4 |
| Example 3 | 85.25 | 0.109 | −3.05 | 94.5 | 85 |

According to the operations, ethanol concentration at the time of rehydration was changed, and the evaluation results of the particle property, and the recovery rate and encapsulation rate of nucleic acid are shown in Table 8.

As shown in Table 8, the encapsulation efficiency of nucleic acid could be enhanced by the addition of ethanol.

TABLE 8
Property evaluation results of lipid nanoparticles−3

| | Size | | | | |
|---|---|---|---|---|---|
| | number | | Z | recovery | |
| | average | | potential | rate | encapsulation |
| | (nm) | PDI | (mV) | (%) | rate (%) |
| Example 2 | 105.5 | 0.140 | −3.05 | 79.9 | 72.4 |
| Example 4 | 99.49 | 0.135 | −4.50 | 84.8 | 78.2 |
| Example 5 | 99.65 | 0.108 | −4.11 | 86.2 | 80.7 |
| Example 3 | 85.25 | 0.109 | −3.05 | 94.5 | 78.6 |
| Example 6 | 80.29 | 0.108 | −3.96 | 95.5 | 74.7 |
| Example 7 | 78.55 | 0.102 | −4.26 | 96.4 | 82.1 |
| Example 8 | 88.88 | 0.153 | −2.67 | 90.6 | 61.6 |
| Example 1 | 144 | 0.253 | −6.4 | 60 | 56 |
| Example 52 | 106 | 0.27 | −6.45 | 61 | 71 |

According to the operations, the type of nucleic acid at the time of rehydration was changed, and the evaluation results of the particle property, and the recovery rate and encapsulation rate of nucleic acid are shown in Table 9.

As shown in the evaluation results of Table 9, nucleic acid could be efficiently encapsulated irrespective of the type of nucleic acid.

TABLE 9
Property evaluation results of lipid nanoparticles-4
(influence of nucleic acid type)

| | Size | | | | |
|---|---|---|---|---|---|
| | number | | Z | recovery | |
| | average | | potential | rate | encapsulation |
| | (nm) | PDI | (mV) | (%) | rate (%) |
| Example 9 | 75.67 | 0.098 | −5.29 | 60.1 | 75.6 |
| Example 6 | 80.29 | 0.108 | −3.96 | 95.5 | 74.7 |
| Example 10 | 136.2 | 0.103 | −3.40 | 55.4 | 93.5 |

According to the operations, the type of phospholipid and the lipid composition were changed, and the evaluation results of the particle property, and the recovery rate and encapsulation rate of nucleic acid are shown in Table 10.

As shown in the evaluation results of Table 10, nucleic acid could be efficiently encapsulated irrespective of the type of phospholipid and the lipid composition.

TABLE 10
Property evaluation results of lipid nanoparticles-5
(influence of phospholipid type and lipid composition)

| | Size | | | | |
|---|---|---|---|---|---|
| | number | | Z | recovery | |
| | average | | potential | rate | encapsulation |
| | (nm) | PDI | (mV) | (%) | rate (%) |
| Example 11 | 95.49 | 0.117 | −4.97 | 88.0 | 72.4 |
| Example 12 | 79.78 | 0.126 | −2.77 | 95.2 | 72.9 |
| Example 13 | 72.87 | 0.142 | −2.10 | 104.6 | 61.4 |
| Example 14 | 86.58 | 0.122 | −5.94 | 94.7 | 70.7 |
| Example 15 | 89.07 | 0.113 | −5.71 | 101.0 | 70.3 |
| Example 16 | 86.39 | 0.132 | −3.65 | 110.5 | 67.8 |
| Example 17 | 88.01 | 0.136 | −2.61 | 117.7 | 63.2 |
| Example 18 | 75 | 0.131 | −5.88 | 87 | 86 |
| Example 19 | 75 | 0.124 | −4.44 | 91 | 85 |
| Example 20 | 68 | 0.128 | −2.41 | 92 | 81 |
| Example 21 | 68 | 0.137 | −6.39 | 87 | 84 |
| Example 22 | 85 | 0.137 | −4.49 | 89 | 82 |
| Example 23 | 81 | 0.140 | −3.39 | 84 | 80 |
| Example 24 | 65 | 0.151 | −3.28 | 91 | 78 |
| Example 25 | 88 | 0.124 | −7.98 | 83 | 82 |
| Example 26 | 82 | 0.123 | −7.53 | 88 | 84 |
| Example 27 | 73 | 0.133 | −6.38 | 88 | 83 |
| Example 28 | 74 | 0.144 | −8.38 | 94 | 84 |
| Example 29 | 69 | 0.137 | −7.35 | 87 | 81 |
| Example 30 | 66 | 0.142 | −6.21 | 89 | 86 |
| Example 31 | 120 | 0.106 | −8.87 | 91 | 91 |
| Example 32 | 112 | 0.101 | −10.6 | 89 | 89 |
| Example 33 | 100 | 0.112 | −4.84 | 84 | 84 |
| Example 34 | 99 | 0.113 | −4.8 | 85 | 85 |
| Example 35 | 102 | 0.104 | −5.95 | 87 | 87 |
| Example 36 | 85 | 0.137 | −2.79 | 79 | 79 |
| Example 37 | 88 | 0.162 | −1.69 | 74 | 74 |
| Example 38 | 106 | 0.122 | −8.41 | 86 | 86 |
| Example 39 | 86 | 0.121 | −6.13 | 84 | 84 |
| Example 7 | 78.55 | 0.102 | −4.26 | 96.4 | 82.1 |
| Example 53 | 109 | 0.092 | −6.49 | 67 | 90 |
| Example 54 | 88 | 0.124 | −6.55 | 73 | 91 |
| Example 55 | 81 | 0.125 | −5.66 | 82 | 87 |
| Example 56 | 95 | 0.103 | −6.52 | 71 | 87 |
| Example 57 | 79 | 0.158 | −5.53 | 75 | 86 |
| Example 58 | 87 | 0.126 | −4.3 | 62 | 92 |
| Example 59 | 97 | 0.123 | −4.09 | 64 | 86 |

According to the operations, the type of ionic lipid was changed, and the evaluation results of the particle property, and the recovery rate and encapsulation rate of nucleic acid are shown in Table 11.

As shown in the evaluation results of Table 11, nucleic acid could be efficiently encapsulated irrespective of the type of ionic lipid.

TABLE 11
Property evaluation results of lipid nanoparticles-6
(influence of ionic lipid type)

| | Size | | | | |
|---|---|---|---|---|---|
| | number | | Z | recovery | |
| | average | | potential | rate | encapsulation |
| | (nm) | PDI | (mV) | (%) | rate (%) |
| Example 40 | 112.7 | 0.477 | 7.14 | 74.4 | 63.1 |
| Example 41 | 64.51 | 0.516 | 5.72 | 87.0 | 70.9 |
| Example 42 | 249.9 | 0.127 | 4.85 | 46.7 | 62.9 |
| Example 43 | 199.0 | 0.802 | 2.51 | 67.3 | 40.1 |
| Example 44 | 42.28 | 0.900 | 5.51 | 85.4 | 79.1 |
| Example 45 | 184.7 | 38.834 | −9.56 | 46.3 | 51.0 |
| Example 46 | 118.9 | 0.173 | −2.56 | 36.4 | 35.8 |
| Example 47 | 91.57 | 0.143 | −2.71 | 53.9 | 51.5 |
| Example 48 | 104.8 | 0.105 | −1.73 | 66.9 | 65.8 |
| Example 49 | 92.55 | 0.150 | −3.44 | 47.3 | 32.0 |
| Example 50 | 92.05 | 0.107 | −0.755 | 65.1 | 61.6 |
| Example 51 | 115.7 | 0.100 | −1.13 | 67.2 | 70.1 |
| Example 60 | 113.4 | 0.223 | −2.82 | 83.2 | 94.3 |
| Example 61 | 117.9 | 0.235 | 0.67 | 99.1 | 93.7 |
| Example 62 | 128.1 | 0.178 | −10.7 | 77.0 | 94.3 |
| Example 63 | 131.4 | 0.188 | −9.18 | 81.7 | 94.2 |
| Example 64 | 173.8 | 0.259 | −3.60 | 123.8 | 98.0 |
| Example 65 | 159.9 | 0.270 | −11.60 | 124.5 | 96.7 |
| Example 66 | 141.7 | 0.209 | −3.41 | 51.2 | 60.9 |
| Example 67 | 113.2 | 0.186 | −5.75 | 68.6 | 75.4 |
| Example 68 | 82.9 | 0.114 | −6.03 | 87.5 | 90.1 |
| Example 69 | 120.6 | 0.138 | −2.37 | 61.8 | 70.3 |
| Example 70 | 124.1 | 0.152 | −2.84 | 62.7 | 69.2 |

### Experimental Example 1

### Evaluation of Expression Intensity and Expression Efficiency Per Cell by EGFP-mRNA-Encapsulating LNP

An LNP solution encapsulating mRNA expressing EGFP was prepared by the method described in Examples.

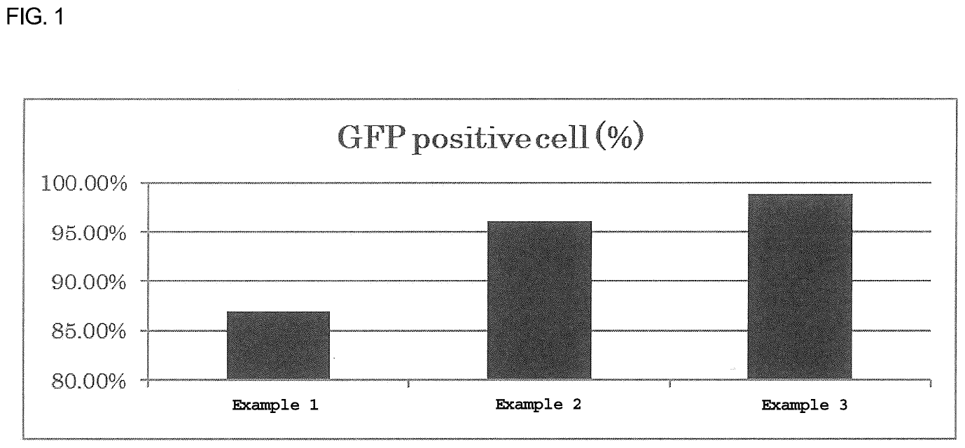

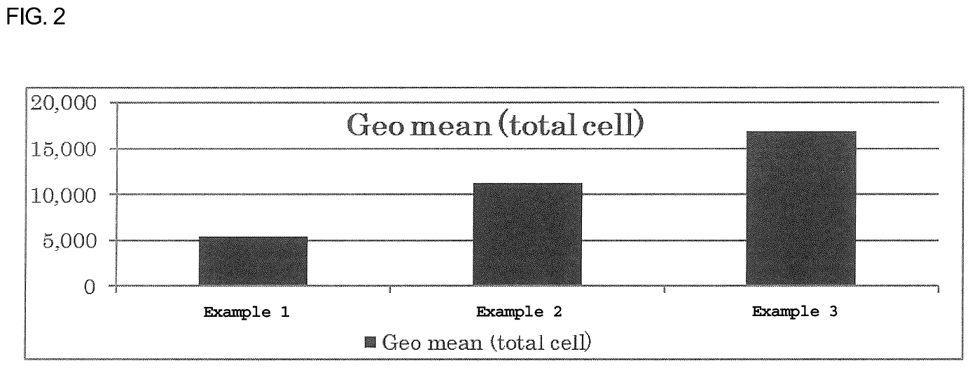

Jurkat cells which are human leukemia T cells were seeded in a 1.2 cm well at 5.0×10⁴ cells/1 mL/well 24 hr before transfection. After 24 hr, the prepared LNP solution was added to 1.2 cm well such that 0.1 μg of mRNA was contained, and the cells were cultured for 24 hr in an incubator. The culture medium was exchanged with FACS buffer (PBS containing 0.5% bovine serum albumin (BSA), 0.1% NaNA), measurement was performed by a flow cytometer (NovoCyte; manufactured by ACEA Biosciences), and transgenic cells were analyzed. The proportion of cells expressing EGFP is shown in FIG. 1, and the expression intensity of EGFP is shown in FIG. 2.

As shown in FIGS. 1 and 2, nucleic acid-encapsulating lipid nanoparticles prepared using the lyophilized composition of the present invention could efficiently introduce genes into cells and could further enhance the uniformity and expression intensity of gene transfer into cells by increasing the concentration of sucrose as a cryoprotectant.

### Experimental Example 2

### Evaluation of Gene Transfer Efficiency into Cells Using Luc-mRNA Encapsulating Lipid Nanoparticles

An LNP solution encapsulating mRNA expressing luciferase was prepared by the method described in Examples.

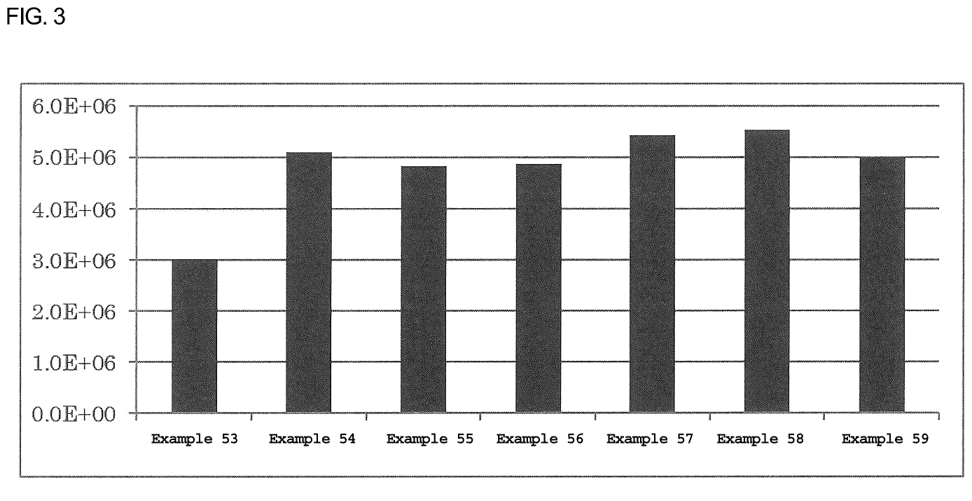

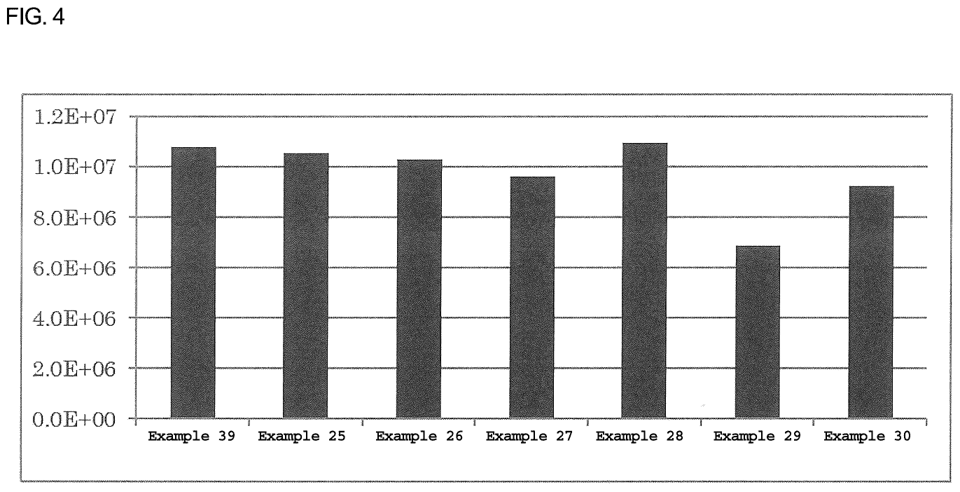

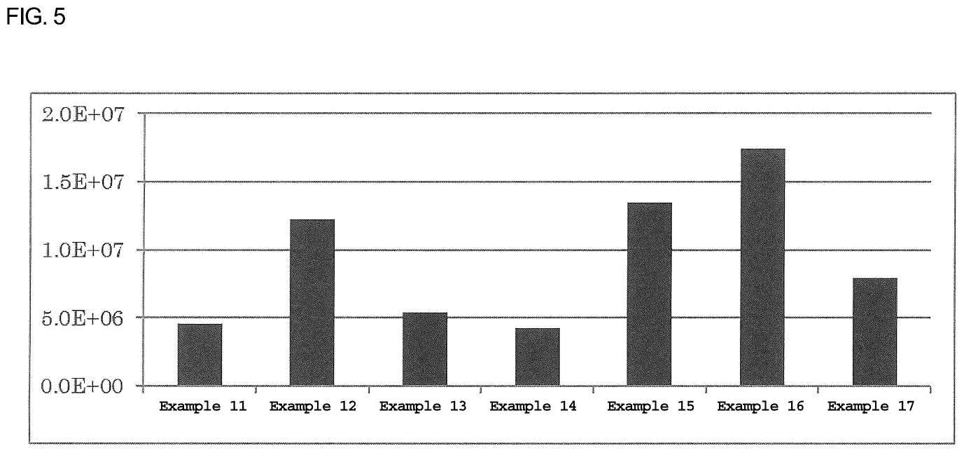

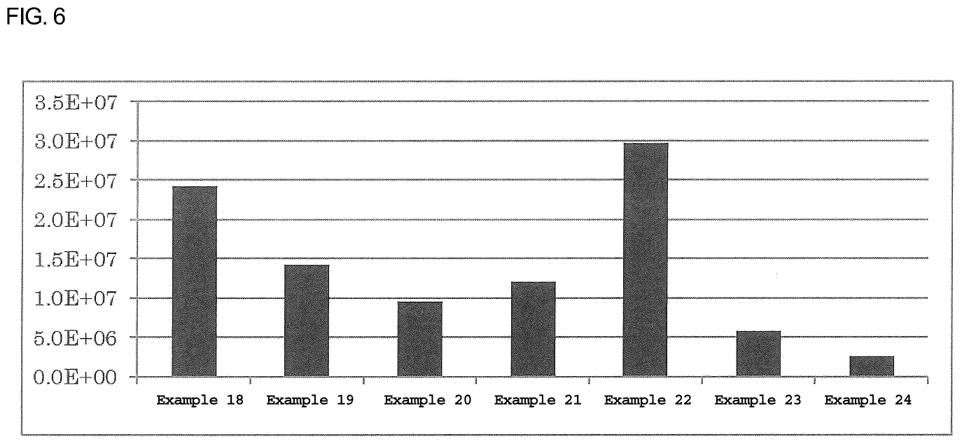

Jurkat cells which are human leukemia T cells were seeded in a 3.5 cm dish at 2.0×10⁵ cells/1.9 mL/Dish 24 hr before transfection. After 24 hr, 100 μL of a D-luciferin-containing medium (RPMI1640) was added to each dish at a final concentration of 0.1 mM. The prepared LNP solution was added thereto such that 0.4 μg of mRNA was contained, and the mixture was set in an incubator luminometer KronosDio. The luminescence intensity of luciferase was measured for 2 min every 3 hr. The cumulative luminescence intensity for 24 or 48 hr was calculated from the obtained time change of expression. The results are shown in FIGS. 3-6.

As shown in FIGS. 3-6, genes could be efficiently introduced into cells by using any of DOPC, POPC, DOPE, and POPE as a phospholipid, and the gene transfer efficiency was the highest when POPE was used.

### Experimental Example 3

### Evaluation of In Vivo mRNA Expression Efficiency in Mouse

An LNP solution encapsulating mRNA expressing erythropoietin was prepared by the method described in Examples.

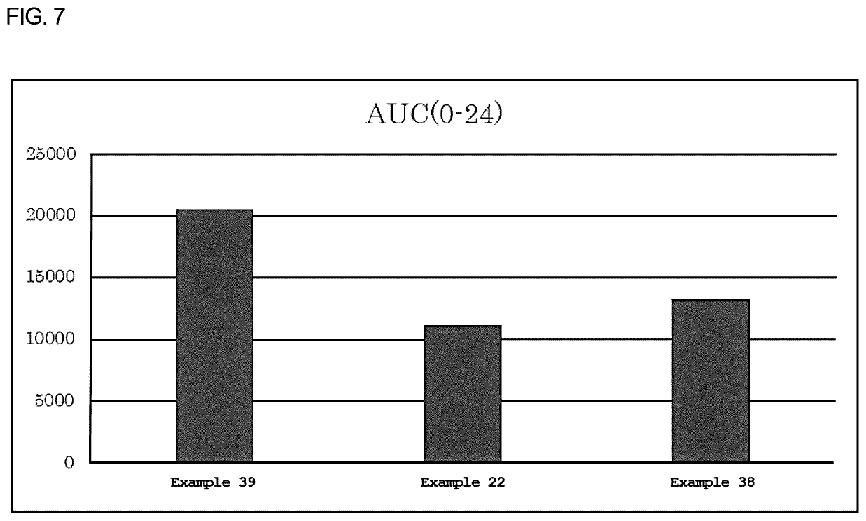

The prepared LNP solution was diluted with PBS such that the concentration of mRNA was 5 μg/mL. The diluted mRNA-encapsulated LNP was administered into the tail vein of 6-week-old female Balb/c mice at 10 μL per 1 g body weight (0.05 mg/kg as dose of mRNA). The blood (15 μL) was collected from the tail vein of the mice 1, 3, 6, 9, 24 hr after the administration. The collected blood was immediately mixed with 0.3 μL of heparin solution (5000 U/5 mL). Each blood sample was centrifuged under centrifugation conditions (25° C., 2000 g, 20 min), and the supernatant was recovered. The concentration of erythropoietin in the supernatant was measured using Mouse Erythropoietin Quantikine ELISA Kit (manufactured by R&D Systems) and by the method described in the protocol of the Kit. The results are shown in FIG. 7.

As shown in FIG. 7, the activity of the particles produced according to Example 39 was the highest, and in vivo mRNA expression in mouse could be confirmed in all of Example 39, Example 22, and Example 38.

### Experimental Example 4

### Evaluation of CTL Activity Using Ovalbumin (OVA; Model Antigen)

The antigen-specific cytotoxic T cell activity (CTL activity) was evaluated using OVA as a model antigen.

LNP encapsulating mRNA that expresses OVA was prepared by the method described in the Examples. The prepared LNP solution was diluted with PBS such that the concentration of mRNA was about 1 μg/mL. The diluted mRNA-encapsulated LNP was administered subcutaneously to the back of the neck of 6-week-old C57BL6/J mouse to achieve 0.1 μg of mRNA per mouse. CTL assay was performed 7 days after administration.

CTL assay: Spleens were collected from 6-week-old C57BL6/J mice not sensitized to the antigen, and they were first loosened in RPMI medium with a 5 mL syringe and then tweezers to prepare splenocytes. The splenocytes were passed through a 40 μm cell strainer, and centrifuged (4° C., 500 g, 5 min). The supernatant was removed, the cell aggregates were suspended in an erythrocyte lysis buffer, and centrifuged again after 5 min (4° C., 500 g, 5 min). The supernatant was removed, RPMI medium was added, and the number of cells was counted. The cells were suspended to 1.0×10⁷ cells/mL and divided into the following two treatment groups.

Target cell group; OVA epitope (SIINFEKL peptide) was added so as to be diluted 400-fold, and the cells were allowed to stand for 30 min and then centrifuged (4° C., 500 g, 5 min). The cells were suspended to 1.0×10⁷ cells/mL and stained with 5 μM CFSE to prepare a target cell group. Control cell group; The cells were suspended to 1.0×10⁷ cells/mL and stained with 0.5 μM CFSE to prepare a control cell group. An equal amount of the solution of the target cell group and the solution of the control cell group was mixed, and a cell mixture having a total of 1.0×10⁷ cells was administered from the tail vein of the mouse immunized as described above. Twenty hours after administration, the spleen of the mouse was collected, and the fluorescence of CFSE was measured with a flow cytometer (NovoCyte; manufactured by ACEA Biosciences). It was confirmed that the measured number of the control cell group showed almost no change throughout the experiment. For each group, the survival ratio of the target cell group to the control cell group was calculated. The 0% of cytotoxic T cell activity was determined from the survival rate of the target cells in PBS-administered mice (non-immune group), and the cytotoxic T cell activity of each sample was calculated from the survival rate of the target cells in immune mice, based on which the OVA specific cytotoxic T cell activity was evaluated.

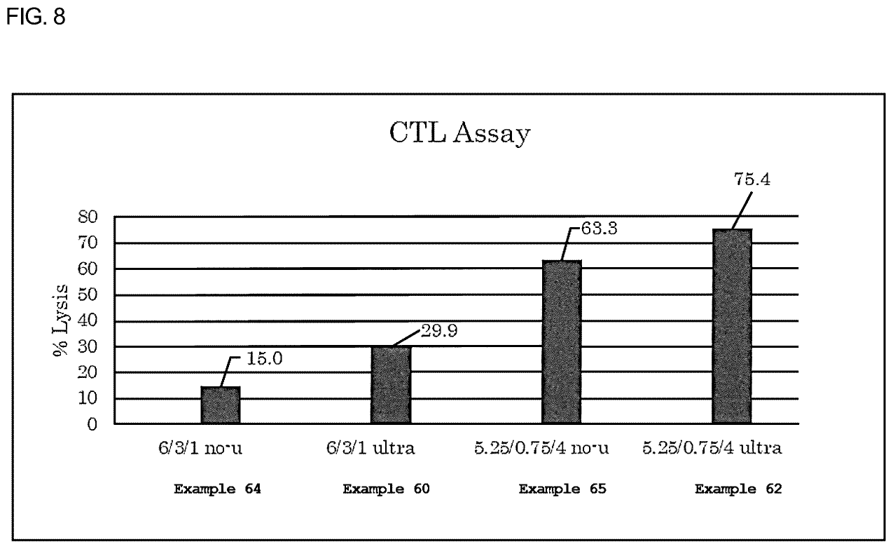

As shown in FIG. 8, while the activity of the particles prepared as in Example 62 and Example 65 was the highest, it was confirmed that the antigen specific cytotoxic T cell activity can be imparted in all of Example 60, Example 62, Example 64, and Example 65.

### Preparation of Lipid Nanoparticles of Comparative Example 4

Particles were prepared according to the following operations by a conventionally-known method that does not undergo a lyophilization step.

At a lipid composition SS-OP/cholesterol/DOPC/DMG-PEG2k=52.5/40/7.5/1.5 (mol) and a ratio of total lipid/mRNA=200 nmol/μg, an ethanol solution of a lipid and malic acid buffer solution of mRNA (pH 3.0, containing 20 mM, 30 mM NaCl) were mixed by NanoAssemblr (flow rate ratio: buffer/lipid=3/1(v/v), total flow rate: 4 mL/min). This solution was diluted with MES buffer (pH 5.5, 20 mM), and replaced with PBS at pH 7.4 while performing ultrafiltration using Amicon. This solution was diluted with PBS such that mRNA was 10 μg/mL as a theoretical value to give lipid nanoparticles.

### Preparation of Lyophilized Composition of Example 71

At a lipid composition SS-OP/cholesterol/DOPC/DMG-PEG2k=52.5/40/7.5/1.5 (mol), an ethanol solution of a lipid and malic acid buffer (pH 3.0, 20 mM) were mixed by NanoAssemblr (flow rate ratio: buffer/lipid=7/1(v/v), total flow rate: 1 mL/min). This solution was diluted with MES buffer (pH 6, 20 mM), and replaced with MES buffer (pH 6, 20 mM) by ultrafiltration using Amicon. The solution was diluted with MES buffer (pH 6, 20 mM) such that the total lipid was 400 nmol/100 μL, and recovered. An equal amount of sucrose solution (sucrose final concentration 160 mg/mL) was added to this solution and mixed with a vortex mixer. 200 μL thereof was dispensed to a vial, and freeze-dried by an EYELA freeze-dryer. For lyophilizing, the solution was first frozen at normal pressure at −40° C. for 3 hr, then the pressure was reduced to 200 mTorr, and the solution was allowed to stand at −40° C. for 20 hr, for 6 hr every 10° C. at −30 to 0° C., and for 3 hr every 10° C. at 10-30° C. After completion of the program, the pressure was returned to normal pressure and the sample was collected.

### Rehydration of Lyophilized Composition of Example 71

200 μL of water containing 2 μg of Luc-mRNA was added to the collected lyophilized sample such that the total lipid/mRNA was 200 nmol/μg, and the mixture was mixed by tapping. It was then incubated at 95° C. for 5 min and neutralized with 200 μL of PBS.

### Preparation of Lyophilized Composition of Example 72

At a lipid composition SS-OP/cholesterol/DOPC/DMG-PEG2k=52.5/40/7.5/1.5 (mol), an ethanol solution of a lipid and malic acid buffer (pH 3.0, 20 mM) were mixed by NanoAssemblr (flow rate ratio: buffer/lipid=7/1(v/v), total flow rate: 16 mL/min). This solution was diluted with MES buffer (pH 6, 20 mM), and replaced with MES buffer (pH 6, 20 mM) by ultrafiltration using Amicon. The solution was diluted with MES buffer (pH 6, 20 mM) such that the total lipid was 400 nmol/100 μL, and recovered. An equal amount of sucrose solution (sucrose final concentration 160 mg/mL) was added to this solution and mixed with a vortex mixer. 200 μL thereof was dispensed to a vial, and freeze-dried by an EYELA freeze-dryer. For lyophilizing, the solution was first frozen at normal pressure at −40° C. for 3 hr, then the pressure was reduced to 200 mTorr, and the solution was allowed to stand at −40° C. for 20 hr, for 6 hr every 10° C. at −30 to 0° C., and for 3 hr every 10° C. at 10-30° C. After completion of the program, the pressure was returned to normal pressure and the sample was collected.

### Rehydration of Lyophilized Composition of Example 72

200 μL of water containing 2 μg of hEPO-mRNA was added to the collected lyophilized sample such that the total lipid/mRNA was 200 nmol/μg, and the mixture was mixed by tapping. It was then incubated at 37° C. for 15 min and neutralized with 200 μL of PBS.

The measurement results of the particle property and nucleic acid encapsulation rate in Comparative Example 4, and Examples 71 and 72 are shown in Table 12.

TABLE 12

| | Size | | |
|---|---|---|---|
| | number | | |
| | average | | encapsulation |
| | (nm) | PDI | rate (%) |
| Comparative | 78.76 | 0.166 | 96.9 |
| Example 4 | | | |
| Example 71 | 163.0 | 0.114 | 100 |
| Example 72 | 113.8 | 0.126 | 93.2 |

### Experimental Example 5

### Evaluation of Gene Expression Activity In Vitro Using HeLa Cells

Using the particles of Comparative Example 4 and Example 71 prepared according to the operations, the intensity of gene expression in cells was evaluated according to the following operation.

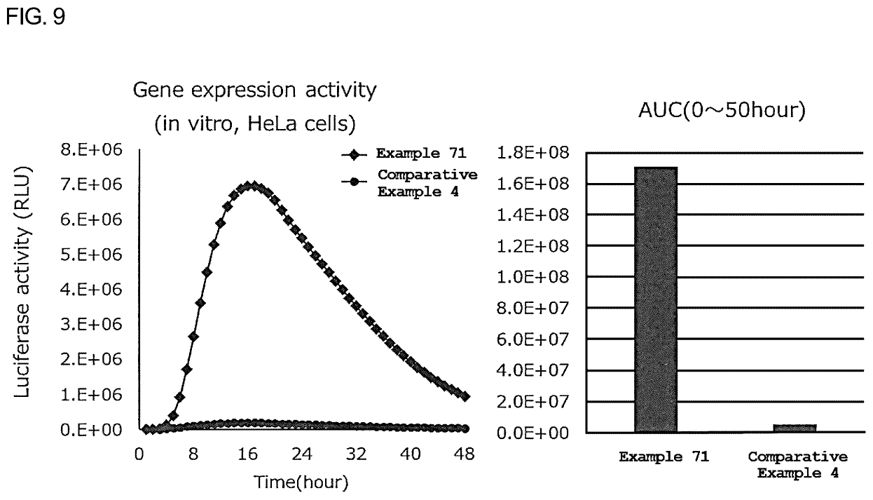

HeLa cells were seeded in a 3.5 cm dish at 5.0×10⁴ cells/1.9 mL/Dish 24 hr before transfection. After 24 hr, a medium (RPMI1640) containing D-luciferin was added to each dish by 100 μL such that the final concentration was 0.1 mM. The prepared LNP solution was added thereto such that 0.4 μg of mRNA was contained, and the mixture was set in an incubator luminometer KronosDio. The luminescence intensity of luciferase was measured for 2 min every 1 hr. The results are shown in FIG. 9. Compared with Comparative Example 4, the nanoparticles of Example 71 showed about 34 times higher gene expression activity in vitro.

### Experimental Example 6

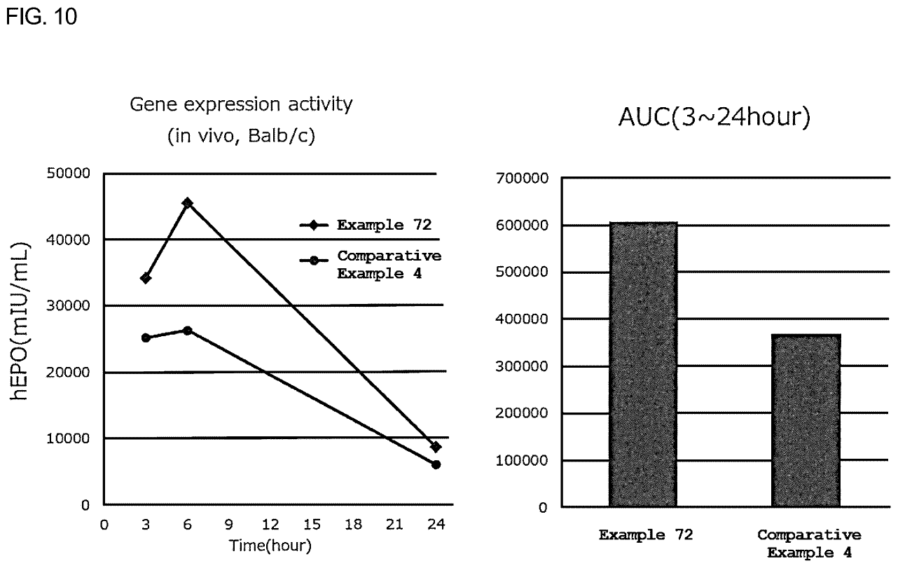

Evaluation of in vivo gene expression activity in mouse Using the particles of Comparative Example 4 and Example 72 prepared according to the operations, the intensity of gene expression in mouse was evaluated in the same manner as in the method of Experimental Example 3. The results are shown in FIG. 10. Compared with Comparative Example 4, the nanoparticles of Example 72 showed about 1.7 times higher gene expression activity in vivo.

### Preparation of lyophilized compositions of Examples 73-94

According to the lipid compositions shown in Table 14, an ethanol solution of a lipid and malic acid buffer (pH 3.0, 20 mM) were mixed by NanoAssemblr (flow rate ratio: buffer/lipid=3/1(v/v), total flow rate: 16 mL/min). This LNP solution was mixed with an equal amount of a sucrose solution (such that the sucrose final concentration was 160 mg/mL), and dispensed to a lipid amount of 400 nmol. This solution was freeze-dried using a VerTis Advantage Plus EL-85 freeze-dryer. For lyophilizing, the solution was first frozen at normal pressure at −55° C. for 14 to 21 hr, then the pressure was reduced to 200 mTorr and the temperature was raised by 10° C. The temperature rise program was set to raise the temperature by 10° C. over 3 hr up to −20° C., raise the temperature by 10° C. over 2 hr above −10° C., and allow the sample to stand at said temperature for 1 hr. When the temperature rose to 30° C., the sample was allowed to stand at said temperature for 3 hr, the pressure was returned to normal pressure, and the sample was collected.

### Rehydration of Lyophilized Compositions of Examples 73-94

Under the conditions shown in Table 14, an aqueous solution of mRNA (using RNase free) was added to a lyophilized composition at a ratio of total lipid/mRNA=200 nmol/μg, mixed by a vortex mixer, and incubated at 95° C. for 5 min. Using Ribogreen (registered trade mark) reagent, the recovery rate and the encapsulation rate of the nucleic acid were measured. The particle size and zeta potential were measured using Zetasizer.

### Measurement of Recovery Rate and Encapsulation Rate

(1) A solution (250 μL) is prepared by diluting a nucleic acid-encapsulating LNP solution 5-fold with DPBS (theoretical value 1 μg/mL).
(2) For calibration curve, a solution (250 μL each) is prepared by diluting an mRNA solution of known concentration with DPBS to an mRNA concentration of 2000 ng/mL, 1000 ng/mL, 500 ng/mL, 250 ng/mL, 125 ng/mL, or 0 ng/mL.
(3) RiboGreen reagent is prepared under the conditions shown in the Table below (amount per well). An amount of (calibration curve number+sample number+1)×2 is prepared for analysis at n=2.

TABLE 13

| | SOLUTION A | SOLUTION B |
|---|---|---|
| | (μL) | (μL) |
| DPBS | 47.75 | 49.75 |
| 10% (w/v) TritonX-100 solution | 2 | 0 |
| (TritonX-100 (Sigma-Aldrich, | | |
| T8787) was diluted with | | |
| Milli-Q water) | | |
| RiboGreen reagent | 0.25 | 0.25 |
| Total | 50 | 50 |

(4) 50 μL of the solution prepared in (1) and (2) is added to each well of a black 96 well plate, and then add 50 μL of SOLUTION A or 50 μL of SOLUTION B is added.
(5) The plate is shaded with aluminum foil and incubated for 5 min on a shaker (100-500 rpm).
(6) The fluorescence is measured with a microplate reader (Excitation: 480 nm, Emission: 520 nm).
(7) The recovery rate and the encapsulation rate are calculated by the following calculation formulas.

$$recovery rate ( \% ) = \frac{concentration obtained from conditions for SOLUTION A addition}{concentration at 100 \% recovery} \times 100$$

$$encapsulation rate ( \% ) = ( 1 - \frac{concentration obtained from SOLUTION B - added sample}{concentration obtained from conditions for SOLUTION A addition} ) \times 100$$

The mRNA sequence used is shown below.
mRNA: CleanCap (registered trade mark) FLuc mRNA (TriLink)

TABLE 14
Compositions of lyophilized products of Examples and rehydration conditions

| | | sterol, type | PEG lipid, | phospholipid, | sucrose | | ethanol | |
|---|---|---|---|---|---|---|---|---|
| | ionic lipid, | and ratio | type and | type and | concentra- | weight | concentra- | type of |
| sample | type and ratio | (molar | ratio (molar | ratio (molar | tion | ratio | tion | nucleic |
| name | (molar ratio) | ratio) | ratio) | ratio) | (mg/mL) | Note 1 | (v/v %) | acid |
| Example | No. 1 | Chol | DMG - PEG2k | DOPC | 160 | 89 | 0 | mRNA |
| 73 | 47.5 | 42.5 | 1.5 | 7.5 | | | | |
| Example | No. 3 | Chol | DMG-PEG2k | DOPC | 160 | 90.4 | 0 | mRNA |
| 74 | 47.5 | 42.5 | 1.5 | 7.5 | | | | |
| Example | No. 4 | Chol | DMG - PEG2k | DOPC | 160 | 86 | 0 | mRNA |
| 75 | 47.5 | 42.5 | 1.5 | 7.5 | | | | |
| Example | No. 5 | Chol | DMG- PEG2k | DOPC | 160 | 83.5 | 0 | mRNA |
| 76 | 47.5 | 42.5 | 1.5 | 7.5 | | | | |
| Example | No. 7 | Chol | DMG- PEG2k | DOPC | 160 | 86 | 0 | mRNA |
| 77 | 47.5 | 42.5 | 1.5 | 7.5 | | | | |
| Example | No. 8 | Chol | DMG-PEG2k | DOPC | 160 | 91.2 | 0 | mRNA |
| 78 | 47.5 | 42.5 | 1.5 | 7.5 | | | | |
| Example | No. 9 | Chol | DMG- PEG2k | DOPC | 160 | 90.5 | 0 | mRNA |
| 79 | 47.5 | 42.5 | 1.5 | 7.5 | | | | |
| Example | No. 10 | Chol | DMG- PEG2k | DOPC | 160 | 84.8 | 0 | mRNA |
| 80 | 47.5 | 42.5 | 1.5 | 7.5 | | | | |
| Example | No. 11 | Chol | DMG - PEG2k | DOPC | 160 | 82.3 | 0 | mRNA |
| 81 | 47.5 | 42.5 | 1.5 | 7.5 | | | | |
| Example | No. 16 | Chol | DMG- PEG2k | DOPC | 160 | 87.6 | 0 | mRNA |
| 82 | 47.5 | 42.5 | 1.5 | 7.5 | | | | |
| Example | No. 21 | Chol | DMG- PEG2k | DOPC | 160 | 89 | 0 | mRNA |
| 83 | 47.5 | 42.5 | 1.5 | 7.5 | | | | |
| Example | No. 22 | Chol | DMG - PEG2k | DOPC | 160 | 88.4 | 0 | mRNA |
| 84 | 47.5 | 42.5 | 1.5 | 7.5 | | | | |
| Example | No. 25 | Chol | DMG- PEG2k | DOPC | 160 | 81.7 | 0 | mRNA |
| 85 | 47.5 | 42.5 | 1.5 | 7.5 | | | | |
| Example | SS-OP | Chol | DMG-PEG2k | DOPC | 160 | 91.7 | 0 | mRNA |
| 86 | 30 | 42.5 | 1.5 | 7.5 | | | | |
| | SS-EC | | | | | | | |
| | 18.5 | | | | | | | |
| Example | No. 30 | Chol | DMG- PEG2k | DOPC | 160 | 133.9 | 0 | mRNA |
| 87 | 47.5 | 42.5 | 1.5 | 7.5 | | | | |
| Example | No. 31 | Chol | DMG - PEG2k | DOPC | 160 | 136.8 | 0 | mRNA |
| 88 | 47.5 | 42.5 | 1.5 | 7.5 | | | | |
| Example | No. 32 | Chol | DMG- PEG2k | DOPC | 160 | 131.1 | 0 | mRNA |
| 89 | 47.5 | 42.5 | 1.5 | 7.5 | | | | |
| Example | No. 33 | Chol | DMG - PEG2k | DOPC | 160 | 133.7 | 0 | mRNA |
| 90 | 47.5 | 42.5 | 1.5 | 7.5 | | | | |
| Example | No. 34 | Chol | DMG- PEG2k | DOPC | 160 | 127.9 | 0 | mRNA |
| 91 | 47.5 | 42.5 | 1.5 | 7.5 | | | | |
| Example | No. 35 | Chol | DMG- PEG2k | DOPC | 160 | 125.5 | 0 | mRNA |
| 92 | 47.5 | 42.5 | 1.5 | 7.5 | | | | |
| Example | No. 36 | Chol | DMG - PEG2k | DOPC | 160 | 133.5 | 0 | mRNA |
| 93 | 47.5 | 42.5 | 1.5 | 7.5 | | | | |
| Example | No. 37 | Chol | DMG - PEG2k | DOPC | 160 | 140 | 0 | mRNA |
| 94 | 47.5 | 42.5 | 1.5 | 7.5 | | | | |

Note 1:
weight ratio of sucrose to total lipid

According to the operations, the type of ionic lipid was changed, and the evaluation results of the particle property, and the recovery rate and encapsulation rate of nucleic acid are shown in Table 15.

As shown in the evaluation results of Table 15, nucleic acid could be efficiently encapsulated irrespective of the type of ionic lipid.

TABLE 15
Property evaluation results of lipid nanoparticles
(influence of ionic lipid type)

| | Size | | | | |
|---|---|---|---|---|---|
| | number | | Z | | |
| | average | | potential | recovery | encapsulation |
| | (nm) | PDI | (mV) | rate (%) | rate (%) |
| Example 73 | 115 | 0.15 | −8.22 | 106 | 74 |
| Example 74 | 105 | 0.14 | −6.76 | 107 | 87 |
| Example 75 | 94 | 0.12 | −5.83 | 102 | 100 |
| Example 76 | 134 | 0.12 | −6.46 | 102 | 82 |
| Example 77 | 96 | 0.60 | −4.66 | 87 | 63 |
| Example 78 | 128 | 0.15 | −8.44 | 100 | 40 |
| Example 79 | 127 | 0.09 | −8.71 | 100 | 50 |
| Example 80 | 135 | 0.31 | −7.18 | 97 | 40 |
| Example 81 | 129 | 0.13 | −6.93 | 100 | 61 |
| Example 82 | 135 | 0.12 | −8.59 | 103 | 35 |
| Example 83 | 64 | 0.37 | −8.65 | 104 | 81 |
| Example 84 | 124 | 0.09 | −9.42 | 101 | 37 |
| Example 85 | 191 | 0.19 | −5.45 | 86 | 47 |
| Example 86 | 93 | 0.09 | −7.01 | 91 | 99 |
| Example 87 | 128 | 0.11 | −2.27 | 106 | 102 |
| Example 88 | 155 | 0.18 | −6.33 | 102 | 101 |
| Example 89 | 111 | 0.09 | 7.26 | 101 | 102 |
| Example 90 | 86 | 0.09 | 7.16 | 94 | 102 |
| Example 91 | 118 | 0.08 | 6.52 | 99 | 101 |
| Example 92 | 115 | 0.09 | 6.53 | 95 | 101 |
| Example 93 | 131 | 0.13 | −2.92 | 106 | 100 |
| Example 94 | 122 | 0.10 | 0.14 | 103 | 100 |

### Experimental Example 7

### Evaluation of Gene Transfer Efficiency into Cells Using Luc-mRNA Encapsulating Lipid Nanoparticles

An LNP solution encapsulating mRNA expressing luciferase was prepared by the method described in Examples.

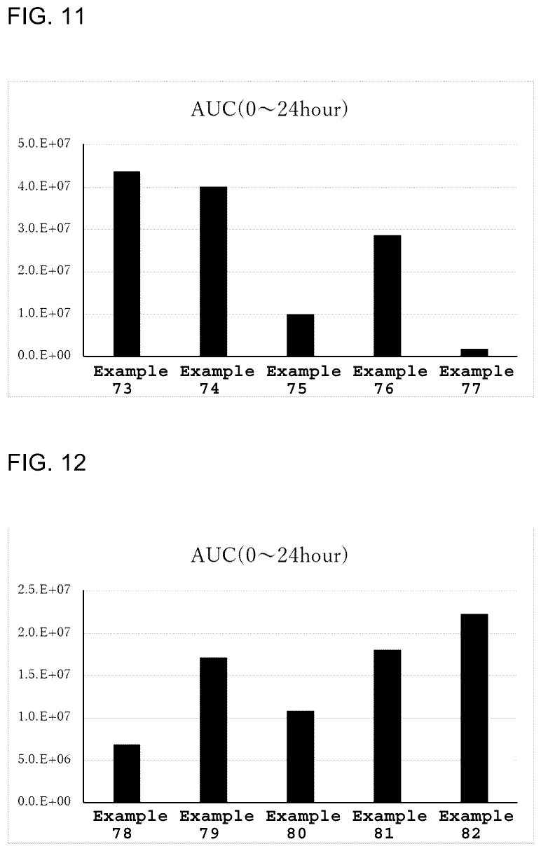

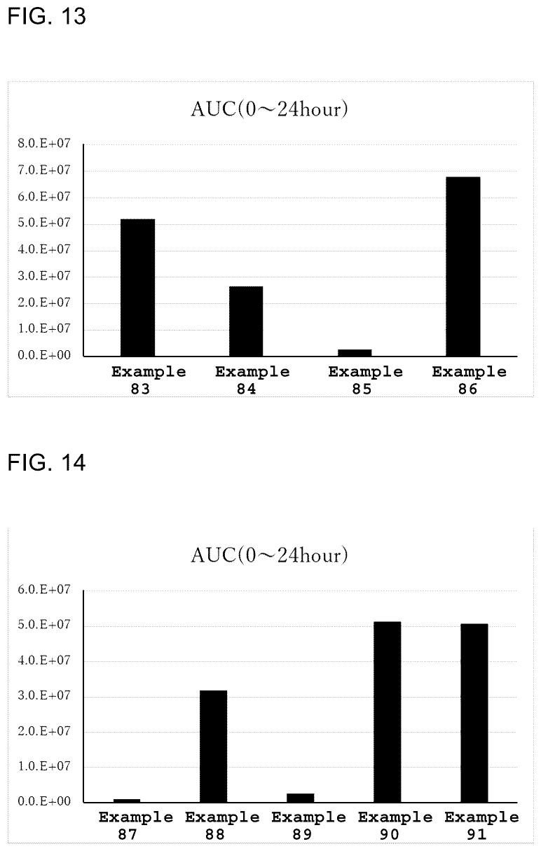

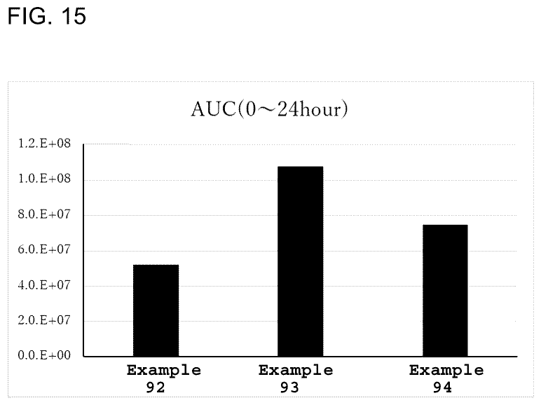

HeLa cells were diluted with a medium (D-MEM) to 5.0×10⁴ cells/mL. D-luciferin was added to a final concentration of 0.1 mM, and the HeLa cell suspension containing D-luciferin was seeded in a 24-well plate at 0.6 mL/well. The prepared LNP solution was added thereto such that 0.03 μg of mRNA was contained, and the mixture was set in an incubator luminometer KronosHT. The luminescence intensity of luciferase was measured for 30 sec every 1 hr. The cumulative luminescence intensity for 24 hr was calculated from the obtained time change of expression. The results are shown in FIGS. 11-15.

As shown in FIGS. 11-15, genes could be efficiently introduced into cells by using each of the ionic lipids, and the gene transfer efficiency was the highest when No. 36 was used.

### [Production Example 1] Synthesis of O-Ph-P4C2

### Acid Anhydridation of Oleic Acid

Oleic acid (manufactured by NOF CORPORATION) (70.0 g, 248 mmol) was dissolved in chloroform (560 g) at room temperature, and the mixture was cooled to 10-15° C. Thereto was added dropwise a suspension of DCC (manufactured by Osaka Synthetic Chemical Laboratories, Inc.) (25.1 g, 121 mmol) dissolved in chloroform (140 g), and the mixture was reacted at 10-25° C. for 2 hr. The reaction solution was filtered, and the filtrate was concentrated by an evaporator. The obtained concentrate was re-dissolved in hexane (210 g), and insoluble material was removed by filtration. The obtained filtrate was concentrated by an evaporator to give oleic anhydride (64.2 g).

### Synthesis of 4-oleoyloxyphenylacetic Acid

Oleic anhydride (43.1 g, 78.9 mmol) and 4-hydroxyphenylacetic acid (manufactured by Tokyo Chemical Industry Co., Ltd.) (6.00 g, 39.4 mmol) were dissolved in chloroform (647 g). Thereto was added DMAP (manufactured by KOEI CHEMICAL CO., LTD.) (1.93 g, 15.8 mmol) and the mixture was reacted at room temperature for 9 hr. The reaction solution was washed twice with 10% aqueous acetic acid solution (216 g) and twice with ion exchange water (216 g). Magnesium sulfate (manufactured by KANTO CHEMICAL CO., INC.) (12.9 g) was added to the organic layer, and the mixture was stirred for 30 min. Magnesium sulfate was filtered off, and the filtrate was concentrated by an evaporator. The concentrate was re-dissolved in hexane (284 g), the insoluble material was filtered off, and the filtrate was extracted 6 times with acetonitrile (168 g). The acetonitrile layer was recovered and concentrated by an evaporator to give a crude product (18.1 g). The obtained crude product (14.5 g) was subjected to column purification to give 4-oleoyloxyphenylacetic acid (3.66 g).

### Synthesis of O-Ph-P4C2

Bis{2-[4-(2-hydroxyethyl)piperidyl]ethyl}disulfide (di-4PE form) (0.350 g, 0.929 mmol) synthesized by the method described in US 2014/0335157 A1, 4-oleoyloxyphenylacetic acid (0.813 g, 1.95 mmol), and DMAP (0.0454 g, 0.372 mmol) were dissolved in chloroform (10.5 g) at room temperature. Thereto was added EDC (0.534 g, 2.79 mmol), and the mixture was reacted at 30-35° C. for 4 hr. The reaction solution was washed twice with 20% brine (7.00 g) and dehydrated using magnesium sulfate (0.350 g). Magnesium sulfate was filtered off, and the filtrate was concentrated in an evaporator to give a crude product (1.10 g). The obtained crude product was subjected to column purification to give O-Ph-P4C2 (0.722 g).

### [Production Example 2] Synthesis of E-Ph-P4C2

### Acid Anhydridation of Succinic Acid D-α-tocopherol

Succinic acid D-α-tocopherol (manufactured by SIGMA-ALDRICH) (70.0 g, 132 mmol) was dissolved in chloroform (560 g) at room temperature, and the mixture was cooled to 10-15° C. Thereto was added dropwise a suspension of DCC (manufactured by Osaka Synthetic Chemical Laboratories, Inc.) (13.7 g, 66 mmol) dissolved in chloroform (140 g), and the mixture was reacted at 10-25° C. for 2 hr. The reaction solution was filtered, and the filtrate was concentrated by an evaporator. The obtained concentrate was re-dissolved in hexane (210 g), and insoluble material was removed by filtration. The obtained filtrate was concentrated by an evaporator to give succinic anhydride D-α-tocopherol (64.2 g).

### Synthesis of 4-(D-α-tocopherol hemisuccinyl)phenylacetic acid

Succinic anhydride D-α-tocopherol (43.1 g, 41.3 mmol) and 4-hydroxyphenylacetic acid (manufactured by Tokyo Chemical Industry Co., Ltd.) (3.13 g, 20.6 mmol) were dissolved in chloroform (647 g). Thereto was added DMAP (manufactured by KOEI CHEMICAL CO., LTD.) (1.01 g, 8.26 mmol) and the mixture was reacted at room temperature for 9 hr. The reaction solution was washed twice with 10% aqueous acetic acid solution (216 g) and twice with ion exchange water (216 g). Magnesium sulfate (manufactured by KANTO CHEMICAL CO., INC.) (12.9 g) was added to the organic layer, and the mixture was stirred for 30 min. Magnesium sulfate was filtered off, and the filtrate was concentrated by an evaporator. The concentrate was re-dissolved in hexane (284 g), the insoluble material was filtered off, and the filtrate was extracted 6 times with acetonitrile (168 g). The acetonitrile layer was recovered and concentrated by an evaporator to give a crude product (17.0 g). The obtained crude product (13.6 g) subjected to column purification to give 4-(D-α-tocopherol hemisuccinyl)phenylacetic acid (3.44 g).

### Synthesis of E-Ph-P4C2

di-4PE form (0.350 g, 0.929 mmol), 4-(D-α-tocopherol hemisuccinyl)phenylacetic acid (1.04 g, 1.95 mmol), and DMAP (0.0454 g, 0.372 mmol) were dissolved in chloroform (10.5 g) at room temperature. Thereto was added EDC (0.534 g, 2.79 mmol), and the mixture was reacted at 30-35° C. for 4 hr. The reaction solution was washed twice with 20% brine (7.00 g) and dehydrated using magnesium sulfate (0.350 g). Magnesium sulfate was filtered off, and the filtrate was concentrated in an evaporator to give a crude product (1.31 g). The obtained crude product was subjected to column purification to give E-Ph-P4C2 (0.860 g).

### INDUSTRIAL APPLICABILITY

According to the present invention, since nucleic acid can be intracellularly introduced with high efficiency, the present invention is useful for nucleic acid medicaments, gene therapy and biochemical experiments.

This application is based on patent application No. 2019-176253 filed in Japan, the contents of which are encompassed in full herein.

1. A lyophilized composition of lipid nanoparticles not comprising a nucleic acid but comprising an ionic lipid, a sterol, a PEG lipid, an acidic buffer component that shows a buffering action at pH 1-6, and a cryoprotectant, wherein a weight ratio of the cryoprotectant and a total lipid is 10:1-1000:1.

2. The lyophilized composition according to claim 1, further comprising a phospholipid.

3. The lyophilized composition according to claim 1, wherein the weight ratio of the cryoprotectant and the total lipid is 30:1-1000:1.

4. The lyophilized composition according to claim 1, wherein a concentration of the cryoprotectant in the composition before lyophilizing is 80-800 mg/mL.

5. The lyophilized composition according to claim 1, wherein a concentration of the cryoprotectant in the composition before lyophilizing is 160-800 mg/mL.

6. The lyophilized composition according to claim 1, wherein the ionic lipid is a compound represented by the formula (1): 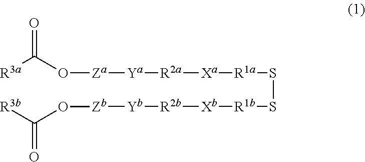
(in the formula (1), R^{1a} and R^{1b} are each independently an alkylene group having 1 to 6 carbon atoms,
X^{a} and X^{b} are each independently an acyclic alkyl tertiary amino group having 1 to 6 carbon atoms and 1 tertiary amino group, or a cyclic alkylene tertiary amino group having 2 to 5 carbon atoms and 1 or 2 tertiary amino groups,
R^{2a} and R^{2b} are each independently an alkylene group having 1 to 8 carbon atoms or an oxydialkylene group having 2 to 8 carbon atoms,
Y^{a} and Y^{b} are each independently an ester bond, an amide bond, a carbamate bond, an ether bond, or a urea bond,
Z^{a} and Z^{b} are each independently a divalent group derived from an aromatic compound having 3 to 16 carbon atoms, at least one aromatic ring, and optionally having a heteroatom,
R^{3a} is
(ia) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group),
(iia) a monovalent group having 10 to 50 carbon atoms and at least two carbonyl groups (excluding a monovalent group containing a residue of a liposoluble vitamin having a hydroxy group and a residue of a sterol derivative having a hydroxy group),
(iiia) a monovalent group represented by the formula (2): 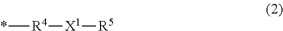
(in the formula (2), * is a bonding position,
R⁴ is an alkylene group having 1 to 10 carbon atoms,
X¹ is a carbamate bond, a carbonate bond, or an amide bond, and
R⁵ is an alkyl group having 1 to 25 carbon atoms, and R⁵ is optionally substituted by a substituent selected from the group consisting of a halogen atom and a hydroxy group),
(iva) a monovalent group represented by the formula (3): 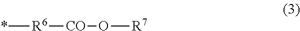
(in the formula (3), * is a bonding position,
R⁶ is an alkylene group having 1 to 10 carbon atoms, and
R⁷ is an alkyl group having 1 to 25 carbon atoms and substituted by at least one halogen atom),
(va) a monovalent group represented by the formula (4): 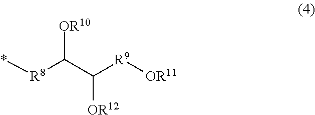
(in the formula (4), * is a bonding position,
R⁸ and R⁹ are each independently an alkylene group having 1 to 10 carbon atoms, an alkenediyl group having 2 to 10 carbon atoms, or an alkynediyl group having 2 to 10 carbon atoms,
R¹⁰ to R¹² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(via) a monovalent group represented by the formula (5): 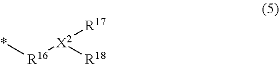
(in the formula (5), * is a bonding position,
X² is a nitrogen atom or a trivalent group represented by the formula (6): 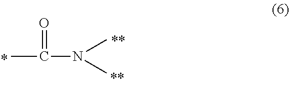
(in the formula (6), * is a bonding position with R¹⁶, and
** is a bonding position with R¹⁷ or R¹⁸),
when X² is a nitrogen atom, R¹⁶ is an alkylene group having 1 to 10 carbon atoms, an alkenediyl group having 2 to 10 carbon atoms, or an alkynediyl group having 2 to 10 carbon atoms, and R¹⁶ is optionally substituted by a substituent selected from the group consisting of a halogen atom and a hydroxy group,
when X² is a trivalent group represented by the formula (6), R¹⁶ is an alkylene group having 1 to 10 carbon atoms, and R¹⁶ is optionally substituted by a substituent selected from the group consisting of a halogen atom and a hydroxy group,
when X² is a nitrogen atom, R¹⁷ and R¹⁸ are each independently an alkyl group having 1 to 10 carbon atoms, an alkenyl group having 2 to 10 carbon atoms, or an alkynyl group having 2 to 10 carbon atoms, and R¹⁷ and R¹⁸ are each independently optionally substituted by a substituent selected from the group consisting of a halogen atom and a hydroxy group, and
when X² is a trivalent group represented by the formula (6), R¹⁷ and R¹⁸ are each independently an alkyl group having 1 to 10 carbon atoms, and R¹⁷ and R¹⁸ are each independently optionally substituted by a substituent selected from the group consisting of a halogen atom and a hydroxy group),
(viia) a monovalent group represented by the formula (7): 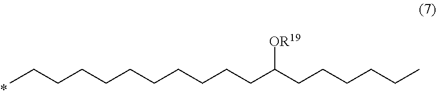
(in the formula (7), * is a bonding position, and
R¹⁹ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group), or a *—CO—R²⁰ group (wherein * is a bonding position, and R²⁰ is an alkyl group having 1 to 9 carbon atoms)),
(viiia) a monovalent group represented by the formula (8): 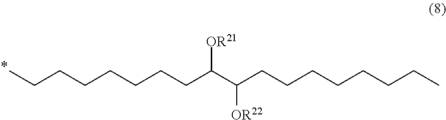
(in the formula (8), * is a bonding position, and
R²¹ and R²² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(ixa) a monovalent group represented by the formula (9): 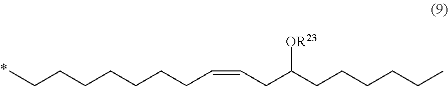
(in the formula (9), * is a bonding position, and
R²³ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(xa) a monovalent group represented by the formula (10): 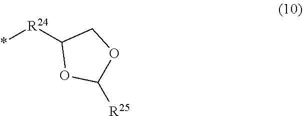
(in the formula (10), * is a bonding position,
R²⁴ is an alkylene group having 1 to 10 carbon atoms, an alkenediyl group having 2 to 10 carbon atoms, or an alkynediyl group having 2 to 10 carbon atoms, and
R²⁵ is an alkyl group having 1 to 30 carbon atoms, an alkenyl group having 2 to 30 carbon atoms, or an alkynyl group having 2 to 30 carbon atoms),
(xia) a monovalent group represented by the formula (11): 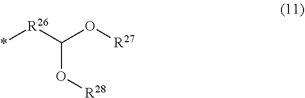
(in the formula (11), * is a bonding position, and
R²⁶ is an alkylene group having 1 to 10 carbon atoms, an alkenediyl group having 2 to 10 carbon atoms, or an alkynediyl group having 2 to 10 carbon atoms, and
R²⁷ and R²⁸ are each independently an alkyl group having 1 to 10 carbon atoms, an alkenyl group having 2 to 10 carbon atoms, or an alkynyl group having 2 to 10 carbon atoms), or
(xiia) a monovalent group represented by the formula (12): 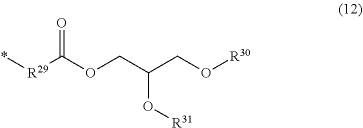
(in the formula (12), * is a bonding position,
R²⁹ is an alkylene group having 1 to 10 carbon atoms, and
R³⁰ and R³¹ are each independently an alkyl group having 1 to 10 carbon atoms),
R^{3b} is
(ib) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group),
(iib) a monovalent group having 10 to 50 carbon atoms and at least two carbonyl groups (excluding a monovalent group containing a residue of a liposoluble vitamin having a hydroxy group and a residue of a sterol derivative having a hydroxy group),
(iiib) a monovalent group represented by the formula (2),
(ivb) a monovalent group represented by the formula (3),
(vb) a monovalent group represented by the formula (4),
(vib) a monovalent group represented by the formula (5),
(viib) a monovalent group represented by the formula (7),
(viiib) a monovalent group represented by the formula (8),
(ixb) a monovalent group represented by the formula (9),
(xb) a monovalent group represented by the formula (10),
(xib) a monovalent group represented by the formula (11),
(xiib) a monovalent group represented by the formula (12),
(xiiib) a monovalent group which is an alkyl group having 1 to 30 carbon atoms, an alkenyl group having 2 to 20 carbon atoms, or an alkynyl group having 2 to 20 carbon atoms, in which one ethylene group in the alkyl group is optionally replaced by one ester bond, or
(xivb) an R^{3c}—CO—(CH₂)ₚ— group (wherein R^{3c} is a residue of a liposoluble vitamin having a hydroxy group or a residue of a sterol derivative having a hydroxy group, and p is an integer of 1 to 8), and
R^{3a} and R^{3b} may be the same or different).

7. The lyophilized composition according to claim 1, wherein the ionic lipid is a compound represented by the formula (2A): 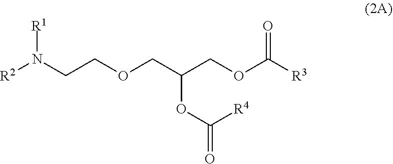
(in the formula (2A), R¹ and R² are each independently an alkyl group having 1 to 6 carbon atoms, or any carbon atoms of R¹ and R² are optionally bonded to each other to form a heterocycle containing nitrogen atom, and
R³ and R⁴ are each independently an aliphatic hydrocarbon group having 1 to 35 carbon atoms optionally substituted by a substituent selected from Substituent Group α,
provided that at least one selected from the group consisting of R³ and R⁴ is an aliphatic hydrocarbon group having 5 to 35 carbon atoms optionally substituted by a substituent selected from Substituent Group α, wherein Substituent Group α consists of a hydroxy group, an alkoxy group, a sulfanyl group, an alkylthio group, and an alkoxycarbonyl group).

8. The lyophilized composition according to claim 1, wherein the cryoprotectant is disaccharide.

9. The lyophilized composition according to claim 1, wherein the cryoprotectant is sucrose.

10. A method for producing a nucleic acid-encapsulating lipid nanoparticle, comprising the following steps:
a) a step of mixing an alcohol solution containing an ionic lipid, a sterol, and a PEG lipid, and an acidic buffer showing a buffering action at pH 1-6 to prepare a suspension of lipid nanoparticles not containing a nucleic acid,
b) a step of mixing the suspension of the lipid nanoparticles not containing a nucleic acid and a cryoprotectant to give a mixture with pH 1-6 and containing the cryoprotectant at 80-800 mg/mL,
c) a step of lyophilizing the mixture obtained in step b to give a lyophilized composition,
d) a step of mixing the lyophilized composition and an aqueous solution containing a nucleic acid and optionally containing alcohol at 0-25 v/v %, and optionally incubating the mixture at 0-95° C. for 0-60 min to give nucleic acid-encapsulating lipid nanoparticles, and
e) a step of exchanging an external aqueous phase of the obtained nucleic acid-encapsulating lipid nanoparticles with a neutral buffer by dialysis, ultrafiltration, or dilution.

11. The method according to claim 10, wherein the step a further comprises a step of exchanging the external aqueous phase with another acidic buffer showing a buffering action at pH 1-6, by dialysis, ultrafiltration, or dilution, after preparing the suspension of lipid nanoparticles.

12. The method according to claim 10, wherein the alcohol solution further comprises a phospholipid in step a.

13. The method according to claim 10, wherein a concentration of the cryoprotectant in the mixture in step b is 160-800 mg/mL.

14. The method according to claim 10, wherein the ionic lipid is a compound represented by the formula (1): 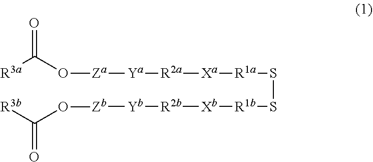
(in the formula (1), R^{1a} and R^{1b} are each independently an alkylene group having 1 to 6 carbon atoms,
X^{a} and X^{b} are each independently an acyclic alkyl tertiary amino group having 1 to 6 carbon atoms and 1 tertiary amino group, or a cyclic alkylene tertiary amino group having 2 to 5 carbon atoms and 1 or 2 tertiary amino groups,
R^{2a} and R^{2b} are each independently an alkylene group having 1 to 8 carbon atoms or an oxydialkylene group having 2 to 8 carbon atoms,
Y^{a} and Y^{b} are each independently an ester bond, an amide bond, a carbamate bond, an ether bond, or a urea bond,
Z^{a} and Z^{b} are each independently a divalent group derived from an aromatic compound having 3 to 16 carbon atoms, at least one aromatic ring, and optionally having a heteroatom,
R^{3a} is
(ia) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group),
(iia) a monovalent group having 10 to 50 carbon atoms and at least two carbonyl groups (excluding a monovalent group containing a residue of a liposoluble vitamin having a hydroxy group and a residue of a sterol derivative having a hydroxy group),
(iiia) a monovalent group represented by the formula (2): 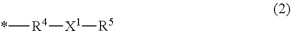
(in the formula (2), * is a bonding position,
R⁴ is an alkylene group having 1 to 10 carbon atoms,
X¹ is a carbamate bond, a carbonate bond, or an amide bond, and
R⁵ is an alkyl group having 1 to 25 carbon atoms, and R⁵ is optionally substituted by a substituent selected from the group consisting of a halogen atom and a hydroxy group),
(iva) a monovalent group represented by the formula (3): 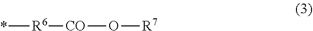
(in the formula (3), * is a bonding position,
R⁶ is an alkylene group having 1 to 10 carbon atoms, and
R⁷ is an alkyl group having 1 to 25 carbon atoms and substituted by at least one halogen atom),
(va) a monovalent group represented by the formula (4): 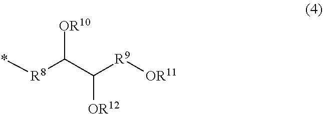
(in the formula (4), * is a bonding position,
R⁸ and R⁹ are each independently an alkylene group having 1 to 10 carbon atoms, an alkenediyl group having 2 to 10 carbon atoms, or an alkynediyl group having 2 to 10 carbon atoms,
R¹⁰ to R¹² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(via) a monovalent group represented by the formula (5): 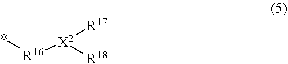
(in the formula (5), * is a bonding position,
X² is a nitrogen atom or a trivalent group represented by the formula (6): 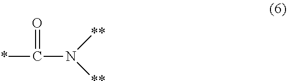
(in the formula (6), * is a bonding position with R¹⁶, and
** is a bonding position with R¹⁷ or R¹⁸),
when X² is a nitrogen atom, R¹⁶ is an alkylene group having 1 to 10 carbon atoms, an alkenediyl group having 2 to 10 carbon atoms, or an alkynediyl group having 2 to 10 carbon atoms, and R¹⁶ is optionally substituted by a substituent selected from the group consisting of a halogen atom and a hydroxy group,
when X² is a trivalent group represented by the formula (6), R¹⁶ is an alkylene group having 1 to 10 carbon atoms, and R¹⁶ is optionally substituted by a substituent selected from the group consisting of a halogen atom and a hydroxy group,
when X² is a nitrogen atom, R¹⁷ and R¹⁸ are each independently an alkyl group having 1 to 10 carbon atoms, an alkenyl group having 2 to 10 carbon atoms, or an alkynyl group having 2 to 10 carbon atoms, and R¹⁷ and R¹⁸ are each independently optionally substituted by a substituent selected from the group consisting of a halogen atom and a hydroxy group, and
when X² is a trivalent group represented by the formula (6), R¹⁷ and R¹⁸ are each independently an alkyl group having 1 to 10 carbon atoms, and R¹⁷ and R¹⁸ are each independently optionally substituted by a substituent selected from the group consisting of a halogen atom and a hydroxy group),
(viia) a monovalent group represented by the formula (7): 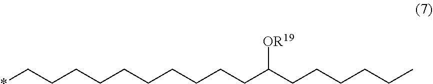
(in the formula (7), * is a bonding position, and
R¹⁹ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group), or a *—CO—R²⁰ group (wherein * is a bonding position, and R²⁰ is an alkyl group having 1 to 9 carbon atoms)),
(viiia) a monovalent group represented by the formula (8): 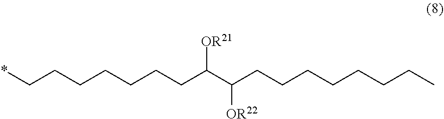
(in the formula (8), * is a bonding position, and
R²¹ and R²² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(ixa) a monovalent group represented by the formula (9): 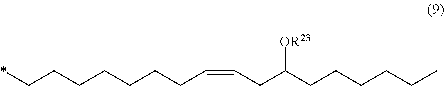
(in the formula (9), * is a bonding position, and
R²³ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(xa) a monovalent group represented by the formula (10): 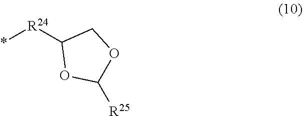
(in the formula (10), * is a bonding position,
R²⁴ is an alkylene group having 1 to 10 carbon atoms, an alkenediyl group having 2 to 10 carbon atoms, or an alkynediyl group having 2 to 10 carbon atoms, and
R²⁵ is an alkyl group having 1 to 30 carbon atoms, an alkenyl group having 2 to 30 carbon atoms, or an alkynyl group having 2 to 30 carbon atoms),
(xia) a monovalent group represented by the formula (11): 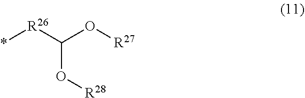
(in the formula (11), * is a bonding position, and
R²⁶ is an alkylene group having 1 to 10 carbon atoms, an alkenediyl group having 2 to 10 carbon atoms, or an alkynediyl group having 2 to 10 carbon atoms, and
R²⁷ and R²⁸ are each independently an alkyl group having 1 to 10 carbon atoms, an alkenyl group having 2 to 10 carbon atoms, or an alkynyl group having 2 to 10 carbon atoms), or
(xiia) a monovalent group represented by the formula (12): 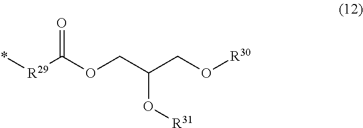
(in the formula (12), * is a bonding position,
R²⁹ is an alkylene group having 1 to 10 carbon atoms, and
R³⁰ and R³¹ are each independently an alkyl group having 1 to 10 carbon atoms),
R^{3b} is
(ib) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group),
(iib) a monovalent group having 10 to 50 carbon atoms and at least two carbonyl groups (excluding a monovalent group containing a residue of a liposoluble vitamin having a hydroxy group and a residue of a sterol derivative having a hydroxy group),
(iiib) a monovalent group represented by the formula (2),
(ivb) a monovalent group represented by the formula (3),
(vb) a monovalent group represented by the formula (4),
(vib) a monovalent group represented by the formula (5),
(viib) a monovalent group represented by the formula (7),
(viiib) a monovalent group represented by the formula (8),
(ixb) a monovalent group represented by the formula (9),
(xb) a monovalent group represented by the formula (10),
(xib) a monovalent group represented by the formula (11),
(xiib) a monovalent group represented by the formula (12),
(xiiib) a monovalent group which is an alkyl group having 1 to 30 carbon atoms, an alkenyl group having 2 to 20 carbon atoms, or an alkynyl group having 2 to 20 carbon atoms, in which one ethylene group in the alkyl group is optionally replaced by one ester bond, or
(xivb) an R^{3c}—CO—(CH₂)ₚ— group (wherein R^{3c} is a residue of a liposoluble vitamin having a hydroxy group or a residue of a sterol derivative having a hydroxy group, and p is an integer of 1 to 8), and
R^{3a} and R^{3b} may be the same or different).

15. The method according to claim 10, wherein the ionic lipid is a compound represented by the formula (2A): 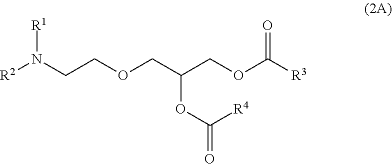
(in the formula (2A), R¹ and R² are each independently an alkyl group having 1 to 6 carbon atoms, or any carbon atoms of R¹ and R² are optionally bonded to each other to form a heterocycle containing nitrogen atom, and
R³ and R⁴ are each independently an aliphatic hydrocarbon group having 1 to 35 carbon atoms optionally substituted by a substituent selected from Substituent Group α,
provided that at least one selected from the group consisting of R³ and R⁴ is an aliphatic hydrocarbon group having 5 to 35 carbon atoms optionally substituted by a substituent selected from Substituent Group α, wherein Substituent Group α consists of a hydroxy group, an alkoxy group, a sulfanyl group, an alkylthio group, and an alkoxycarbonyl group).

16. The method according to claim 10, wherein the cryoprotectant is disaccharide.

17. The method according to claim 10, wherein the cryoprotectant is sucrose.

18. A method for producing a lyophilized composition of lipid nanoparticles, comprising a step of lyophilizing a composition of lipid nanoparticles not containing a nucleic acid but containing an ionic lipid, a sterol, a PEG lipid, an acidic buffer that shows a buffering action at pH 1-6, and a cryoprotectant at 80-800 mg/mL.

19. The method according to claim 18, wherein the composition of lipid nanoparticles further comprises a phospholipid.

20. The method according to claim 18, wherein a concentration of the cryoprotectant in the composition before lyophilizing is 160-800 mg/mL.

21. The method according to claim 18, wherein the ionic lipid is a compound represented by the formula (1): 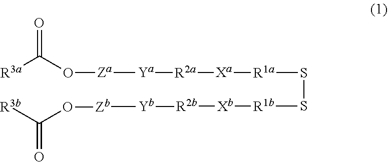
(in the formula (1), R^{1a} and R^{1b} are each independently an alkylene group having 1 to 6 carbon atoms,
X^{a} and X^{b} are each independently an acyclic alkyl tertiary amino group having 1 to 6 carbon atoms and 1 tertiary amino group, or a cyclic alkylene tertiary amino group having 2 to 5 carbon atoms and 1 or 2 tertiary amino groups,
R^{2a} and R^{2b} are each independently an alkylene group having 1 to 8 carbon atoms or an oxydialkylene group having 2 to 8 carbon atoms,
Y^{a} and Y^{b} are each independently an ester bond, an amide bond, a carbamate bond, an ether bond, or a urea bond,
Z^{a} and Z^{b} are each independently a divalent group derived from an aromatic compound having 3 to 16 carbon atoms, at least one aromatic ring, and optionally having a heteroatom,
R^{3a} is
(ia) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group),
(iia) a monovalent group having 10 to 50 carbon atoms and at least two carbonyl groups (excluding a monovalent group containing a residue of a liposoluble vitamin having a hydroxy group and a residue of a sterol derivative having a hydroxy group),
(iiia) a monovalent group represented by the formula (2): 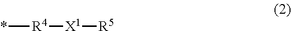
(in the formula (2), * is a bonding position,
R⁴ is an alkylene group having 1 to 10 carbon atoms,
X¹ is a carbamate bond, a carbonate bond, or an amide bond, and
R⁵ is an alkyl group having 1 to 25 carbon atoms, and R⁵ is optionally substituted by a substituent selected from the group consisting of a halogen atom and a hydroxy group),
(iva) a monovalent group represented by the formula (3): 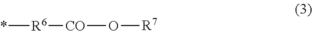
(in the formula (3), * is a bonding position,
R⁶ is an alkylene group having 1 to 10 carbon atoms, and
R⁷ is an alkyl group having 1 to 25 carbon atoms and substituted by at least one halogen atom),
(va) a monovalent group represented by the formula (4): 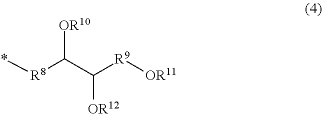
(in the formula (4), * is a bonding position,
R⁸ and R⁹ are each independently an alkylene group having 1 to 10 carbon atoms, an alkenediyl group having 2 to 10 carbon atoms, or an alkynediyl group having 2 to 10 carbon atoms,
R¹⁰ to R¹² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(via) a monovalent group represented by the formula (5): 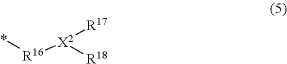
(in the formula (5), * is a bonding position,
X² is a nitrogen atom or a trivalent group represented by the formula (6): 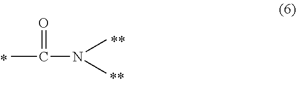
(in the formula (6), * is a bonding position with R¹⁶, and
** is a bonding position with R¹⁷ or R¹⁸),
when X² is a nitrogen atom, R¹⁶ is an alkylene group having 1 to 10 carbon atoms, an alkenediyl group having 2 to 10 carbon atoms, or an alkynediyl group having 2 to 10 carbon atoms, and R¹⁶ is optionally substituted by a substituent selected from the group consisting of a halogen atom and a hydroxy group,
when X² is a trivalent group represented by the formula (6), R¹⁶ is an alkylene group having 1 to 10 carbon atoms, and R¹⁶ is optionally substituted by a substituent selected from the group consisting of a halogen atom and a hydroxy group,
when X² is a nitrogen atom, R¹⁷ and R¹⁸ are each independently an alkyl group having 1 to 10 carbon atoms, an alkenyl group having 2 to 10 carbon atoms, or an alkynyl group having 2 to 10 carbon atoms, and R¹⁷ and R¹⁸ are each independently optionally substituted by a substituent selected from the group consisting of a halogen atom and a hydroxy group, and
when X² is a trivalent group represented by the formula (6), R¹⁷ and R¹⁸ are each independently an alkyl group having 1 to 10 carbon atoms, and R¹⁷ and R¹⁸ are each independently optionally substituted by a substituent selected from the group consisting of a halogen atom and a hydroxy group),
(viia) a monovalent group represented by the formula (7): 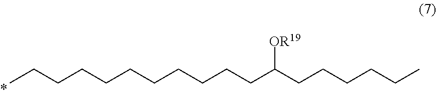
(in the formula (7), * is a bonding position, and
R¹⁹ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group), or a *—CO—R²⁰ group (wherein * is a bonding position, and R²⁰ is an alkyl group having 1 to 9 carbon atoms)),
(viiia) a monovalent group represented by the formula (8): 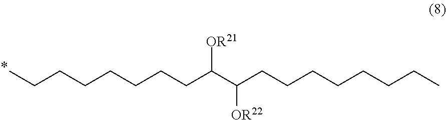
(in the formula (8), * is a bonding position, and
R²¹ and R²² are each independently a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(ixa) a monovalent group represented by the formula (9): 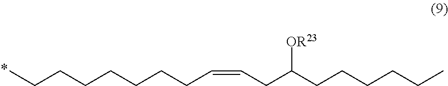
(in the formula (9), * is a bonding position, and
R²³ is a hydrogen atom, a benzyl group, or a *—Si(R¹³)(R¹⁴)(R¹⁵) group (wherein * is a bonding position, and R¹³ to R¹⁵ are each independently an alkyl group having 1 to 4 carbon atoms or a phenyl group)),
(xa) a monovalent group represented by the formula (10): 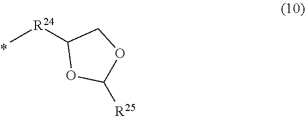
(in the formula (10), * is a bonding position,
R²⁴ is an alkylene group having 1 to 10 carbon atoms, an alkenediyl group having 2 to 10 carbon atoms, or an alkynediyl group having 2 to 10 carbon atoms, and
R²⁵ is an alkyl group having 1 to 30 carbon atoms, an alkenyl group having 2 to 30 carbon atoms, or an alkynyl group having 2 to 30 carbon atoms),
(xia) a monovalent group represented by the formula (11): 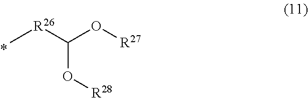
(in the formula (11), * is a bonding position, and
R²⁶ is an alkylene group having 1 to 10 carbon atoms, an alkenediyl group having 2 to 10 carbon atoms, or an alkynediyl group having 2 to 10 carbon atoms, and
R²⁷ and R²⁸ are each independently an alkyl group having 1 to 10 carbon atoms, an alkenyl group having 2 to 10 carbon atoms, or an alkynyl group having 2 to 10 carbon atoms), or
(xiia) a monovalent group represented by the formula (12): 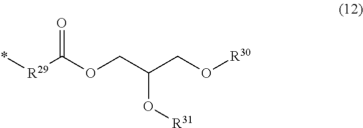
(in the formula (12), * is a bonding position,
R²⁹ is an alkylene group having 1 to 10 carbon atoms, and
R³⁰ and R³¹ are each independently an alkyl group having 1 to 10 carbon atoms),
R^{3b} is
(ib) a monovalent group having 10 to 50 carbon atoms, one carbonyl group, and at least one unsaturated bond selected from the group consisting of an olefinic carbon-carbon double bond and a carbon-carbon triple bond (excluding a monovalent group containing a residue of a liposoluble vitamin having a hydroxy group and a residue of a sterol derivative having a hydroxy group),
(iib) a monovalent group having 10 to 50 carbon atoms and at least two carbonyl groups (excluding a monovalent group containing a residue of a liposoluble vitamin having a hydroxy group and a residue of a sterol derivative having a hydroxy group),
(iiib) a monovalent group represented by the formula (2),
(ivb) a monovalent group represented by the formula (3),
(vb) a monovalent group represented by the formula (4),
(vib) a monovalent group represented by the formula (5),
(viib) a monovalent group represented by the formula (7),
(viiib) a monovalent group represented by the formula (8),
(ixb) a monovalent group represented by the formula (9),
(xb) a monovalent group represented by the formula (10),
(xib) a monovalent group represented by the formula (11),
(xiib) a monovalent group represented by the formula (12),
(xiiib) a monovalent group which is an alkyl group having 1 to 30 carbon atoms, an alkenyl group having 2 to 20 carbon atoms, or an alkynyl group having 2 to 20 carbon atoms, in which one ethylene group in the alkyl group is optionally replaced by one ester bond, or
(xivb) an R^{3c}—CO—(CH₂)ₚ— group (wherein R^{3c} is a residue of a liposoluble vitamin having a hydroxy group or a residue of a sterol derivative having a hydroxy group, and p is an integer of 1 to 8), and
R^{3a} and R^{3b} may be the same or different).

22. The method according to claim 18, wherein the ionic lipid is a compound represented by the formula (2A): 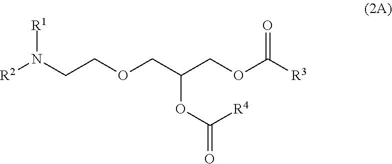
(in the formula (2A), R¹ and R² are each independently an alkyl group having 1 to 6 carbon atoms, or any carbon atoms of R¹ and R² are optionally bonded to each other to form a heterocycle containing nitrogen atom, and
R³ and R⁴ are each independently an aliphatic hydrocarbon group having 1 to 35 carbon atoms optionally substituted by a substituent selected from Substituent Group α,
provided that at least one selected from the group consisting of R³ and R⁴ is an aliphatic hydrocarbon group having 5 to 35 carbon atoms optionally substituted by a substituent selected from Substituent Group α, wherein Substituent Group α consists of a hydroxy group, an alkoxy group, a sulfanyl group, an alkylthio group, and an alkoxycarbonyl group).

23. The method according to claim 18, wherein the cryoprotectant is disaccharide.

24. The method according to claim 18, wherein the cryoprotectant is sucrose.

25. A nucleic acid-introducing agent comprising the lyophilized composition according to claim 1.

26. A method for introducing a nucleic acid into a cell in vitro, comprising bringing the nucleic acid-introducing agent according to claim 25 encapsulating the nucleic acid into contact with the cell.

27. A method for introducing a nucleic acid into a target cell, comprising administering the nucleic acid-introducing agent according to claim 25 encapsulating the nucleic acid to a living organism to allow for delivery of the nucleic acid to the cell.

28. A method for introducing a nucleic acid into a cell, comprising the following steps:
a) a step of mixing an alcohol solution containing an ionic lipid, a sterol, and a PEG lipid, and an acidic buffer showing a buffering action at pH 1-6 to prepare a suspension of lipid nanoparticles not containing a nucleic acid,
b) a step of mixing the suspension of the lipid nanoparticles not containing a nucleic acid and a cryoprotectant to give a mixture with pH 1-6 and containing the cryoprotectant at 80-800 mg/mL,
c) a step of lyophilizing the mixture obtained in step b to give a lyophilized composition,
d) a step of mixing the lyophilized composition and an aqueous solution containing the nucleic acid and optionally containing alcohol at 0-25 v/v %, and optionally incubating the mixture at 0-95° C. for 0-60 min to give nucleic acid-encapsulating lipid nanoparticles,
e) a step of exchanging an external aqueous phase of the obtained nucleic acid-encapsulating lipid nanoparticles with a neutral buffer by dialysis, ultrafiltration, or dilution, and
f) a step of bringing the obtained nucleic acid-encapsulating lipid nanoparticles into contact with the cell in vitro.

29. The method according to claim 28, wherein the step a further comprises a step of exchanging the external aqueous phase with another acidic buffer showing a buffering action at pH 1-6, by dialysis, ultrafiltration, or dilution, after preparing the suspension of lipid nanoparticles.

30. A method for introducing a nucleic acid into a target cell, comprising the following steps:
a) a step of mixing an alcohol solution containing an ionic lipid, a sterol, and a PEG lipid, and an acidic buffer showing a buffering action at pH 1-6 to prepare a suspension of lipid nanoparticles not containing a nucleic acid,
b) a step of mixing the suspension of the lipid nanoparticles not containing a nucleic acid and a cryoprotectant to give a mixture with pH 1-6 and containing the cryoprotectant at 80-800 mg/mL,
c) a step of lyophilizing the mixture obtained in step b to give a lyophilized composition,
d) a step of mixing the lyophilized composition and an aqueous solution containing the nucleic acid and optionally containing alcohol at 0-25 v/v %, and optionally incubating the mixture at 0-95° C. for 0-60 min to give nucleic acid-encapsulating so lipid nanoparticles,
e) a step of exchanging an external aqueous phase of the obtained nucleic acid-encapsulating lipid nanoparticles with a neutral buffer by dialysis, ultrafiltration, or dilution, and
f) a step of administering the obtained nucleic acid-encapsulating lipid nanoparticles to a living organism to allow for delivery of the nucleic acid to the target cell.

31. The method according to claim 30, wherein the step a further comprises a step of exchanging the external aqueous phase with another acidic buffer showing a buffering action at pH 1-6, by dialysis, ultrafiltration, or dilution, after preparing the suspension of lipid nanoparticles.
